(19) United States
(12) Patent Application Publication
(10) Pub. No.: US 2026/0009776 A1
(43) Pub. Date: Jan. 8, 2026

(54) AUTOMATED GRADING PROCESSES AND ODOR SENSING IN AGRICULTURAL APPLICATIONS

(71) Applicant: REALMFIVE, Inc., Lincoln, NE (US)

(72) Inventors: Brant Burkey, Denton, NE (US); Mateusz Mittek, Lincoln, NE (US); David Reznicek, Omaha, NE (US); Steve R. Tippery, Gretna, NE (US); Josh Behlen, Lincoln, NE (US); Dan Pickerill, Milford, NE (US)

(21) Appl. No.: 19/259,801

(22) Filed: Jul. 3, 2025

Related U.S. Application Data
(60) Provisional application No. 63/667,504, filed on Jul. 3, 2024.
(60) Provisional application No. 63/729,139, filed on Dec. 6, 2024.
(60) Provisional application No. 63/759,956, filed on Feb. 18, 2025.
(60) Provisional application No. 63/784,784, filed on Apr. 7, 2025.

(51) Int. Cl. G01N 33/02; G01N 1/22; G01N 1/24; G01N 1/26

(52) U.S. Cl. CPC G01N 33/025; G01N 1/2226; G01N 1/24; G01N 1/26

(57) ABSTRACT

A grading system is disclosed. The grading system may include sampling probes. The grading system may include sensors, to collect information of a material within a container, wherein the sampling probes deliver the material to the sensors. The grading system may include a support structure to position the sampling probes above the container. The grading system may include motors coupled to the sampling probes to actuate the sampling probes. The grading system may include a controller including processors to execute a set of program instructions stored in memory to cause the processors to cause the motors to actuate the sampling probes such that the sampling probes are inserted into the container at one or more locations to collect the information of the material within the container, receive the information collected by the sensors and grade the material within the container based on the information collected by the sensors.

### CROSS-REFERENCE TO RELATED APPLICATIONS

The present application claims the benefit under 35 U.S.C. § 119(e) of U.S. Provisional Application Ser. No. 63/667,504, filed Jul. 3, 2024, entitled AUTOMATED GRADING PROCESSES AND ODOR SENSING IN AGRICULTURAL APPLICATIONS; U.S. Provisional Application Ser. No. 63/729,139, filed Dec. 6, 2024, entitled AUTOMATED GRADING PROCESSES AND ODOR SENSING IN AGRICULTURAL APPLICATIONS; U.S. Provisional Application Ser. No. 63/759,956, filed Feb. 18, 2025, entitled AUTOMATED GRADING AND SENSING PROCESSES IN AGRICULTURAL APPLICATIONS; and U.S. Provisional Application Ser. No. 63/784,784, filed Apr. 7, 2025, entitled AUTOMATED COMMODITIES GRADING, AUTO GRAIN PROBING, ODOR SENSING, AND FUTURES FUNDS IN AGRICULTURAL APPLICATIONS, which are all incorporated herein by reference in their entireties.

### TECHNICAL FIELD

This disclosure relates broadly to crop management, and, more particularly, to crop grading.

### BACKGROUND

Traditional grain grading and processing typically uses extensive manpower and manual grading of the grain. Such a technique may be hindered by lack of labor, which may be further exacerbated by grain grading and processing occurring sparsely populated rural areas. Additionally, grain grading performed by humans may see inconsistencies and bias from the grades. Grading facilities may also need to stay open long hours (e.g., during harvest seasons), which may increase costs due to overtime.

Therefore, it is desirable to provide systems and methods that address the above deficiencies.

### SUMMARY

A grading system is disclosed, in accordance with one or more embodiments of the present disclosure. In embodiments, the grading system includes one or more sampling probes. In embodiments, the grading system includes one or more sensors, wherein the one or more sensors are configured to collect information of a material within a container, wherein the one or more sampling probes deliver the material to the one or more sensors. In embodiments, the grading system includes a support structure, wherein the support structure is configured to position the one or more sampling probes above the container. In embodiments, the grading system includes one or more motors, wherein the one or more motors are coupled to the one or more sampling probes in order to actuate the one or more sampling probes. In embodiments, the grading system includes a controller communicatively coupled to the one or more sensors and the one or more motors, the controller including one or more processors configured to execute a set of program instructions stored in memory, the set of program instructions configured to cause the one or more processors to: cause the one or more motors to actuate the one or more sampling probes such that the one or more sampling probes are inserted into the container at one or more locations to collect the information of the material within the container; receive the information collected by the one or more sensors; and grade the material within the container based on the information collected by the one or more sensors.

A grading method is disclosed, in accordance with one or more embodiments of the present disclosure. In embodiments, the grading method includes positioning one or more sampling probes over a container containing a material. In embodiments, the grading method includes inserting the one or more sampling probes into the material. In embodiments, the grading method includes collecting information of the material within the container with one or more sensors after the one or more sampling probes deliver the material to the one or more sensors. In embodiments, the grading method includes grading the material within the container based on the information collected by the one or more sensors.

A continuous grading system is disclosed, in accordance with one or more embodiments of the present disclosure. In embodiments, the continuous grading system includes a weighing device, wherein the weighing device weighs a material to calculate a bulk density. In embodiments, the continuous grading system includes an optics device, wherein the optics device is configured to capture one or more images of the material as the material passes through the optics device. In embodiments, the continuous grading system includes a moisture sensing device, wherein the moisture sensing device determines a moisture content of the material based on a cross section of the material. In embodiments, the continuous grading system includes an odor sensing device, wherein the odor sensing device determines an odor profile of the material. In embodiments, the continuous grading system includes one or more valves, wherein the one or more valves are configured to control movement of the material, wherein the one or more valves are configured to allow the material to flow without an external input. In embodiments, the continuous grading system includes a controller communicatively coupled to the weighing device, the optics device, the moisture sensing device, an odor sensing device, and the one or more valves, the controller including one or more processors configured to execute a set of program instructions stored in memory, the set of program instructions configured to cause the one or more processors to: receive weight of the material, the one or more images of the material, the moisture content of the material, and the odor profile of the material and grade the material based on the one or more images of the material, the moisture content of the material, and the odor profile of the material.

A continuous grading method is disclosed, in accordance with one or more embodiments of the present disclosure. In embodiments, the continuous grading method includes weighing a material to determine a bulk density of the material. In embodiments, the continuous grading method includes capturing one or more images of the material. In embodiments, the continuous grading method includes measuring a moisture content of the material based on a cross section of the material. In embodiments, the continuous grading method includes determining an odor profile of the material. In embodiments, the continuous grading method includes allowing the material to flow continuously and without an external input between the weighing of the material, the capturing one or more images of the material, and the measuring the moisture content of the material. In embodiments, the continuous grading method includes grading the material based on at least the one or more images of the material, the moisture content of the material, and the odor profile of the material.

A separation device is disclosed, in accordance with one or more embodiments of the present disclosure. In embodiments, the separation device includes a first tube. In embodiments, the separation device includes a second tube, wherein the first tube and the second tube are coupled such that the second tube enters the first tube at an angle relative to the first tube, wherein the second tube introduces a combination of a material and a foreign material to the first tube. In embodiments, the separation device includes a fan, wherein the fan is configured to blow a gas at a selected velocity, wherein the gas separates the material and the foreign material, wherein the gas directs the foreign material in one direction of the first tube and the material falls in a second direction of the first tube.

A weighing device is disclosed, in accordance with one or more embodiments of the present disclosure. In embodiments, the weighing device includes a fixed volume, wherein the fixed volume is configured to be filled with a material such that the material entirely fills the fixed volume without compressing the material. In embodiments, the weighing device includes a bottom valve, wherein the bottom valve is configured to prevent the material from exiting the fixed volume. In embodiments, the weighing device includes one or more load cells, wherein the one or more load cells are coupled to the fixed volume such that the one or more load cells weigh the material when the material entirely fills the fixed volume without being compressed.

An optics device is disclosed, in accordance with one or more embodiments of the present disclosure. In embodiments, the optics device includes a chamber, wherein the chamber is configured to allow a material to fall through the chamber. In embodiments, the optics device includes a valve, wherein the valve is configured to be opened to a predetermined amount depending on a type of material to be imaged. In embodiments, the optics device includes one or more cameras, wherein the one or more cameras are coupled to the chamber, wherein the one or more cameras are configured to image the material inside the chamber. In embodiments, the optics device includes lighting, wherein the lighting is positioned within the chamber and are configured to illuminate within the chamber, wherein the lighting surrounds the one or more cameras and the one or more lights are synchronized to illuminate the chamber with the one or more cameras.

A valve is disclosed, in accordance with one or more embodiments of the present disclosure. In embodiments, the valve includes a plurality of blades. In embodiments, the valve includes an actuator, wherein the actuator is configured to actuate the plurality of blades. In embodiments, the valve includes a housing, wherein the housing includes a central hole and a ring structure, wherein the actuator is configured to actuate the plurality of blades between an open position and a closed position, wherein when in the closed position the plurality of blades blocks the central hole, wherein when in the open position, the plurality of blades is positioned within the ring structure and leave the central hole at least partially unobstructed.

A futures index generation system is disclosed, in accordance with one or more embodiments of the present disclosure. In embodiments, the futures index generation system includes a network of grading devices, wherein each grading device in the network of grading devices is configured to independently grade a material. In embodiments, the futures index generation system includes a controller communicatively coupled to the network of grading devices, the controller including one or more processors configured to execute a set of program instructions stored in memory, the set of program instructions configured to cause the one or more processors to: receive the grade of the material from each of the network of grading devices as grade data; aggregate the grade data; generate an index, wherein the index is based on an average value of the material, wherein the average value of the material is based at least partially on the grade data; and construct a futures fund based on the index.

A futures index generation method is disclosed, in accordance with one or more embodiments of the present disclosure. In embodiments, the futures index generation method includes grading a material with a network of grading devices, wherein each grading device in the network of grading devices is configured to independently grade the material. In embodiments, the futures index generation method includes receiving the grade of the material from each of the network of grading devices as grade data. In embodiments, the futures index generation method includes aggregating the grade data. In embodiments, the futures index generation method includes generating an index, wherein the index is based on an average value of the material, wherein the average value of the material is based at least partially on the grade data. In embodiments, the futures index generation method includes constructing a futures fund based on the index.

A sorting system is disclosed, in accordance with one or more embodiments of the present disclosure. In embodiments, the sorting system includes a conveyor, wherein the conveyor is configured to move a material. In embodiments, the sorting system includes an automated sorting device, wherein the automated sorting device is configured to sort the material based on one or more criteria. In embodiments, the sorting system includes one or more optical sensors positioned along the conveyor to image the material as it passes the one or more optical sensors. In embodiments, the sorting system includes one or more odor sensors positioned along the conveyor to capture an odor profile of the material as it passes the one or more odor sensors. In embodiments, the sorting system includes a controller communicatively coupled to the automated sorting device, the one or more optical sensors, and the one or more odor sensors, the controller including one or more processors configured to execute a set of program instructions stored in memory, the set of program instructions configured to cause the one or more processors to receive the images of the material from the one or more optical sensors and the odor profile of the material from the one or more odor sensors; check for inconsistencies in the material based on the images and the odor profile; and cause the automated sorting device to sort the material based on the inconsistencies.

A mass estimating device is disclosed, in accordance with one or more embodiments of the present disclosure. In embodiments, the mass estimating device includes a tube, wherein the tube contains a material. In embodiments, the mass estimating device includes a blower, wherein the blower is configured to blow a gas at a selected velocity in order to deflect the material within the tube. In embodiments, the mass estimating device includes one or more sensors, wherein the one or more sensors detect the material deflected within the tube and measure a displacement of the material. In embodiments, the mass estimating device includes a controller communicatively coupled to the blower and the one or more sensors, the controller including one or more processors configured to execute a set of program instructions stored in memory, the set of program instructions configured to cause the one or more processors to: receive the displacement of the material from the one or more sensors and estimate mass of the material based on the displacement of the material.

A mass estimating method is disclosed, in accordance with one or more embodiments of the present disclosure. In embodiments, the mass estimating method includes blowing a gas at a selected velocity with a blower in order to deflect a material within a tube. In embodiments, the mass estimating method includes detecting the material deflected within the tube with one or more sensors. In embodiments, the mass estimating method includes measuring a displacement of the material within the tube with one or more sensors. In embodiments, the mass estimating method includes estimating mass of the material based on the displacement of the material.

### BRIEF DESCRIPTION OF THE DRAWINGS

The numerous advantages of the disclosure may be better understood by those skilled in the art by reference to the accompanying figures.

FIG. 1A illustrates a block diagram of a grading system, in accordance with one or more embodiments of the present disclosure.

FIG. 1B illustrates a grading system, in accordance with one or more embodiments of the present disclosure.

FIG. 1C illustrates a grading system, in accordance with one or more embodiments of the present disclosure.

FIG. 1D illustrates a grading system, in accordance with one or more embodiments of the present disclosure.

FIG. 1E illustrates a grading system with more than one sampling probe, where the sampling probes are in a stored state, in accordance with one or more embodiments of the present disclosure.

FIG. 1F illustrates a grading system with more than one sampling probe, where the sampling probes are in a stored state, in accordance with one or more embodiments of the present disclosure.

FIG. 1G illustrates a grading system with more than one sampling probe, where the sampling probes are in a deployed state, in accordance with one or more embodiments of the present disclosure.

FIG. 1H illustrates a grading system with more than one sampling probe, where the sampling probes are in a deployed state, in accordance with one or more embodiments of the present disclosure.

FIG. 1I illustrates a grading system with more than one sampling probe, where the sampling probes are in a deployed state, in accordance with one or more embodiments of the present disclosure.

FIG. 2A illustrates rejection of material due to damage, in accordance with one or more embodiments of the present disclosure.

FIG. 2B illustrates rejection of material due to odor, in accordance with one or more embodiments of the present disclosure.

FIG. 2C illustrates rejection of material due to the material not being correct, in accordance with one or more embodiments of the present disclosure.

FIG. 2D illustrates approval of material due to criteria being met, in accordance with one or more embodiments of the present disclosure.

FIG. 3 illustrates a flow diagram of a grading method, in accordance with one or more embodiments of the present disclosure.

FIG. 4A illustrates a block diagram of a continuous grading system, in accordance with one or more embodiments of the present disclosure.

FIG. 4B illustrates a continuous grading system, in accordance with one or more embodiments of the present disclosure.

FIG. 4C illustrates a continuous grading system, in accordance with one or more embodiments of the present disclosure.

FIG. 4D illustrates material entering the continuous grading system, in accordance with one or more embodiments of the present disclosure.

FIG. 5 illustrates a separation device, in accordance with one or more embodiments of the present disclosure.

FIG. 6A illustrates a weighing device, in accordance with one or more embodiments of the present disclosure.

FIG. 6B illustrates a side view of a weighing device, in accordance with one or more embodiments of the present disclosure.

FIG. 6C illustrates a cross-sectional view of the weighing device, in accordance with one or more embodiments of the present disclosure.

FIG. 6D illustrates the weighing device with load cells exposed, in accordance with one or more embodiments of the present disclosure.

FIG. 7A illustrates a side view of an optics device, in accordance with one or more embodiments of the present disclosure.

FIG. 7B illustrates a cross-sectional view of the optics device, in accordance with one or more embodiments of the present disclosure.

FIG. 8A illustrates an isometric view of a valve, in accordance with one or more embodiments of the present disclosure.

FIG. 8B illustrates a cross-sectional view of the valve, in accordance with one or more embodiments of the present disclosure.

FIG. 8C illustrates an exploded view of the valve, in accordance with one or more embodiments of the present disclosure.

FIG. 9A illustrates a side view of a moisture sensing device, in accordance with one or more embodiments of the present disclosure.

FIG. 9B illustrates an internal view of the moisture sensing device, in accordance with one or more embodiments of the present disclosure.

FIG. 9C illustrates a capacitance-style moisture sensing device, in accordance with one or more embodiments of the present disclosure.

FIG. 10 illustrates a flow diagram of a continuous grading method, in accordance with one or more embodiments of the present disclosure.

FIG. 11 illustrates a block diagram of a futures index generation system, in accordance with one or more embodiments of the present disclosure.

FIG. 12 illustrates a flow diagram of a futures index generation method, in accordance with one or more embodiments of the present disclosure.

FIG. 13 illustrates a block diagram of a sorting system, in accordance with one or more embodiments of the present disclosure.

FIG. 14 illustrates a mass estimating device, in accordance with one or more embodiments of the present disclosure.

FIG. 15 illustrates a flow diagram of a mass estimating method, in accordance with one or more embodiments of the present disclosure.

FIG. 16A illustrates an odor sensing device without a cover, in accordance with one or more embodiments of the present disclosure.

FIG. 16B illustrates the odor sensing device with the cover, in accordance with one or more embodiments of the present disclosure.

### DETAILED DESCRIPTION

Reference will now be made in detail to the subject matter disclosed, which is illustrated in the accompanying drawings. The present disclosure has been particularly shown and described with respect to certain embodiments and specific features thereof. The embodiments set forth herein are taken to be illustrative rather than limiting. It should be readily apparent to those of ordinary skill in the art that various changes and modifications in form and detail may be made without departing from the spirit and scope of the disclosure.

Embodiments of the present disclosure are directed to automated grading systems and methods. An automated grain sampling apparatus for trucks may significantly streamline the quality control process at grain handling facilities. This system may be integrated with the truck scale to automatically detect when a trailer is in position for sampling. Using robotic arms or extendable probes equipped with sensors, the apparatus may be able to penetrate the grain mass at various depths and locations within the trailer to collect representative samples. Advanced sensors and software algorithms may control the sampling mechanism to ensure comprehensive coverage of different sections of the load. These samples may then be conveyed to an on-site laboratory or an integrated analysis unit for immediate testing of moisture content, test weight, foreign material, damage assessment, odor sensing, and other key quality parameters. The entire process—from positioning the probe to collecting and analyzing the sample—may be automated and managed by a central computer system that records the data and provides real-time feedback. This automation may improve the accuracy and efficiency of grain quality assessment while reducing labor demands and the potential for human error.

Implementing an automated grain sampling apparatus in truck trailers may offer significant advantages by augmenting existing on-site labor and allowing workers to focus on more value-added tasks. This technological integration may enhance operational efficiency and productivity in several ways. Automated sampling may reduce the time needed to collect and analyze grain samples, potentially accelerating the entire process. This may enable faster turnaround times and improved throughput, allowing facilities to handle larger volumes without increasing labor requirements. By minimizing human error, automation may result in more accurate and consistent sampling outcomes. Reliable grain quality data may support better-informed decisions regarding storage, processing, and sales.

With repetitive and time-consuming sampling tasks automated, on-site labor may be redirected toward more strategic responsibilities such as equipment maintenance, process optimization, quality control, and customer service—activities that may contribute more directly to a facility's efficiency and profitability. Automated systems may reduce the need for manual involvement in potentially hazardous situations, such as climbing on truck trailers or handling heavy equipment. This may help lower the risk of workplace injuries and improve overall employee safety. These systems may integrate seamlessly with data management platforms to deliver real-time insights into grain quality and operational performance. Such data may be used to track trends, enhance decision-making, and optimize processes. By automating routine tasks, facilities may reduce labor costs related to grain sampling. Additionally, improved accuracy and operational efficiency may lead to better resource utilization and lower levels of waste.

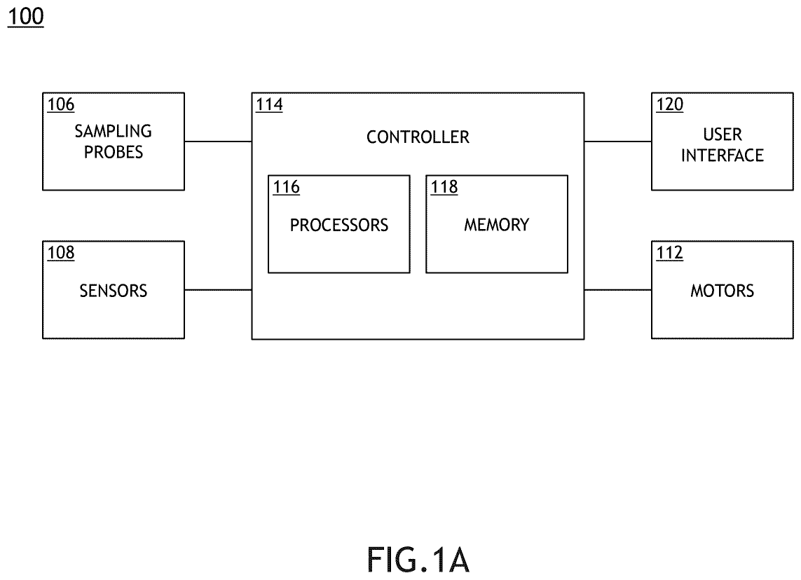

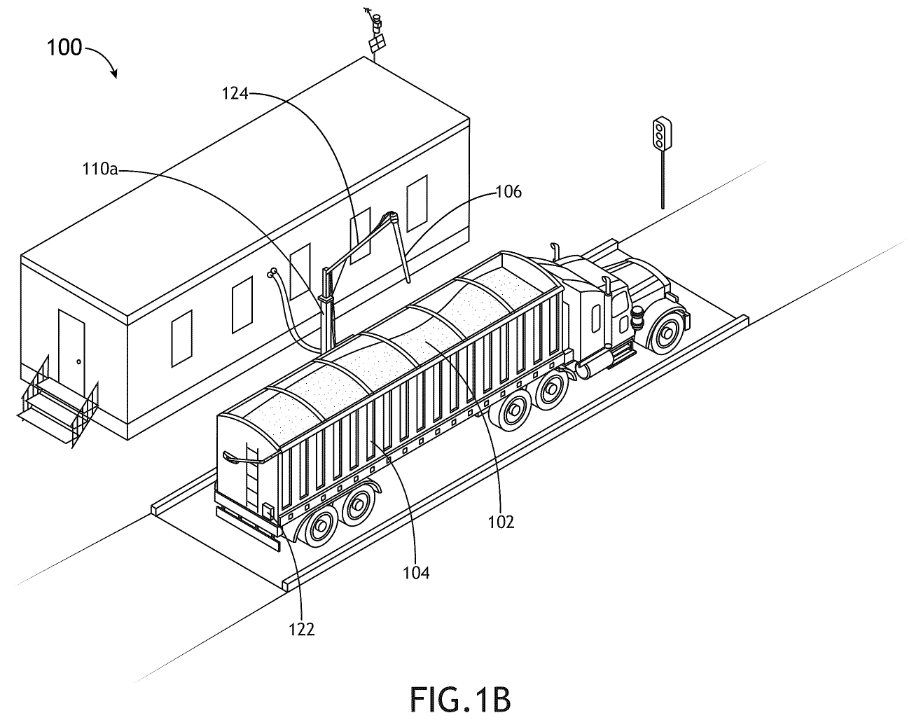

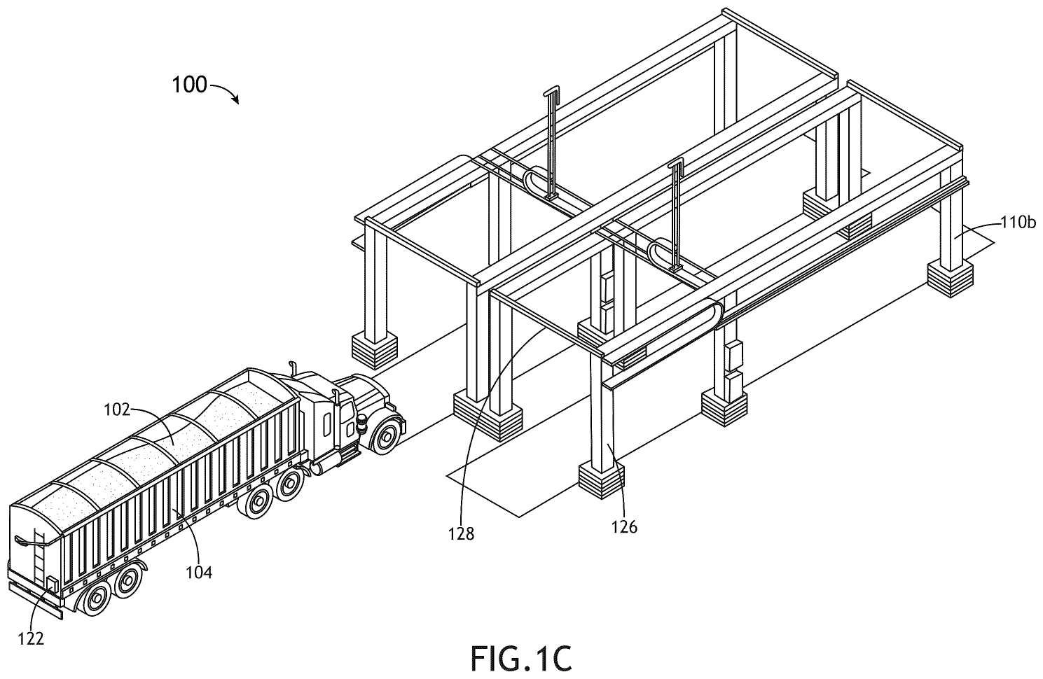

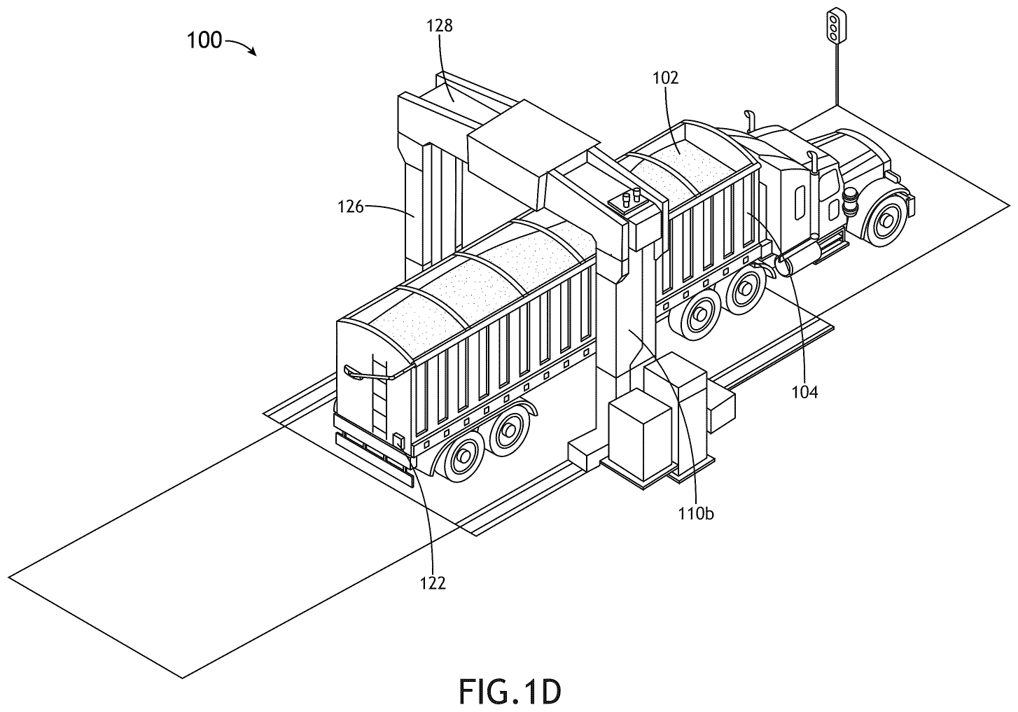

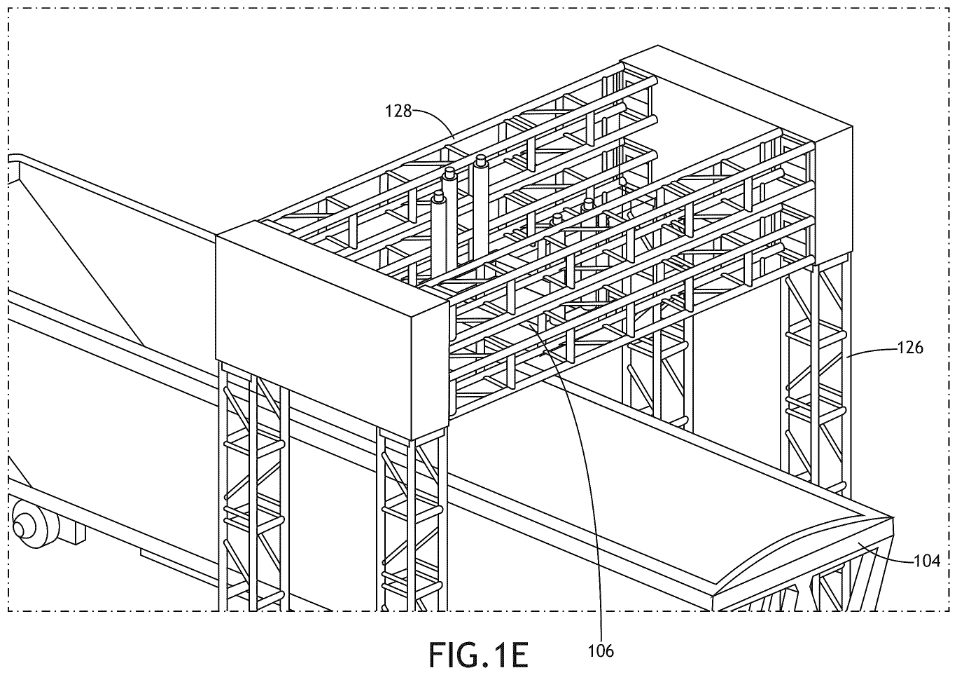

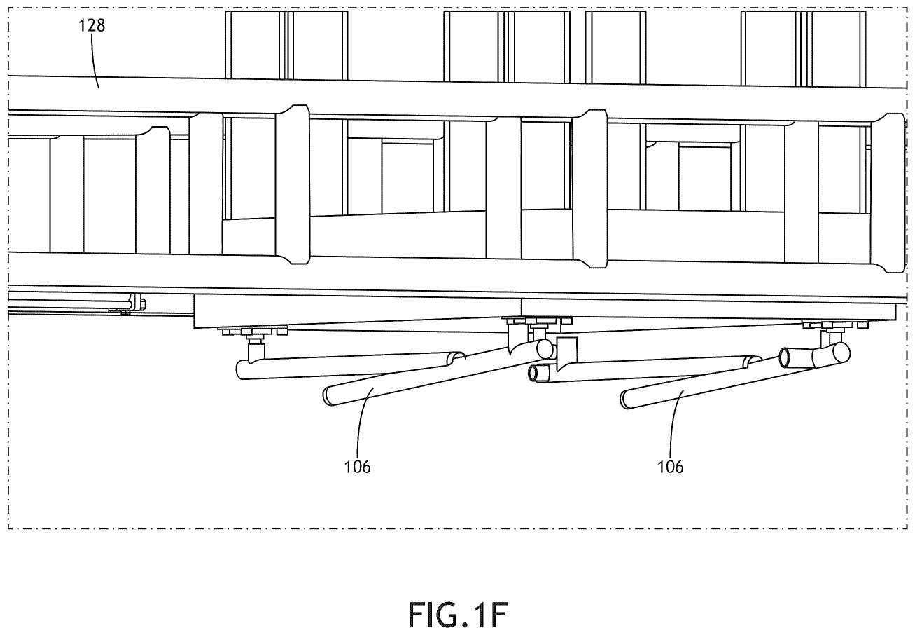

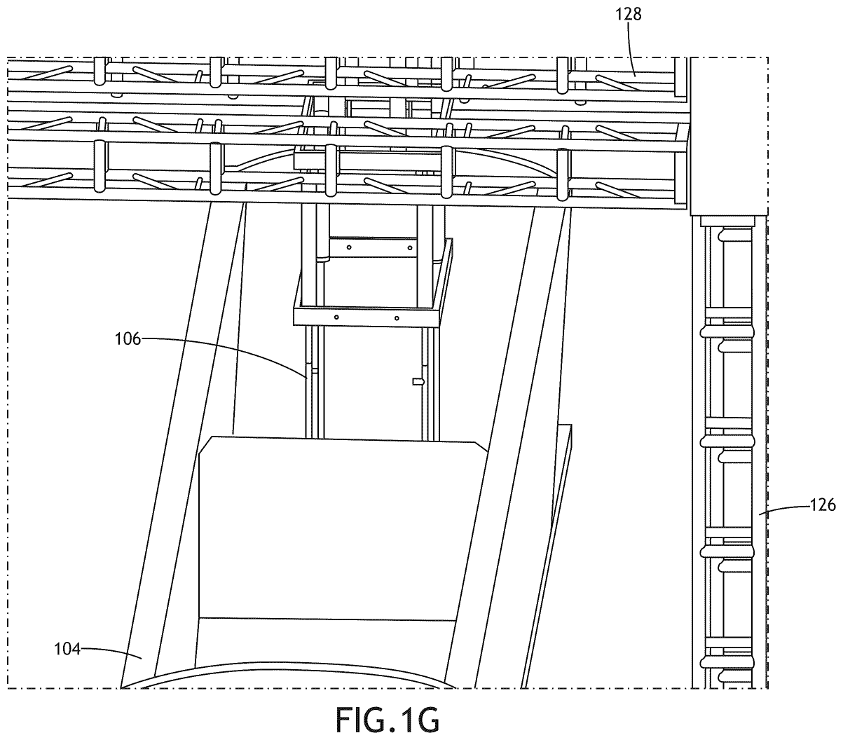

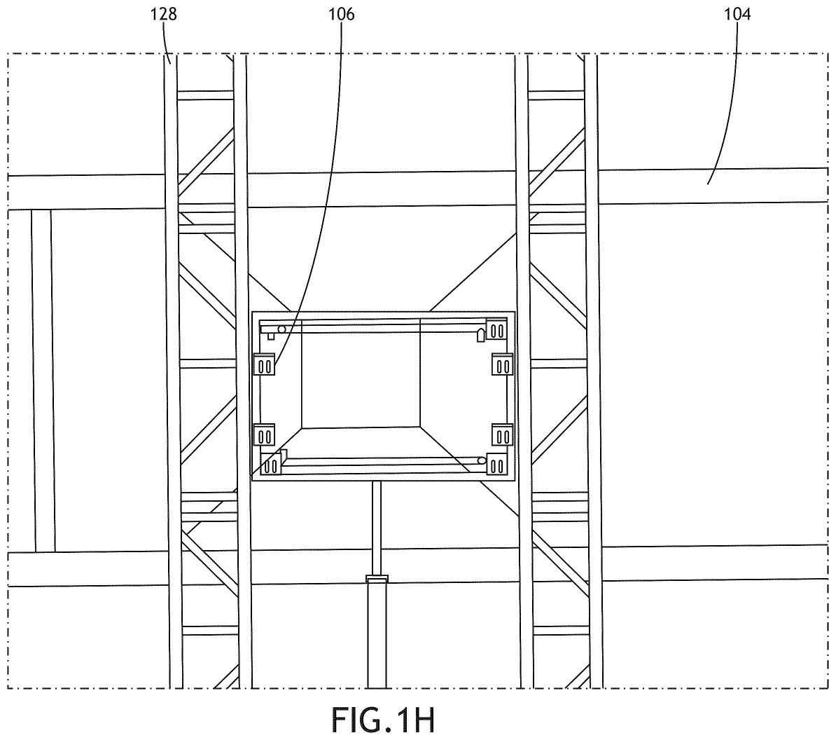

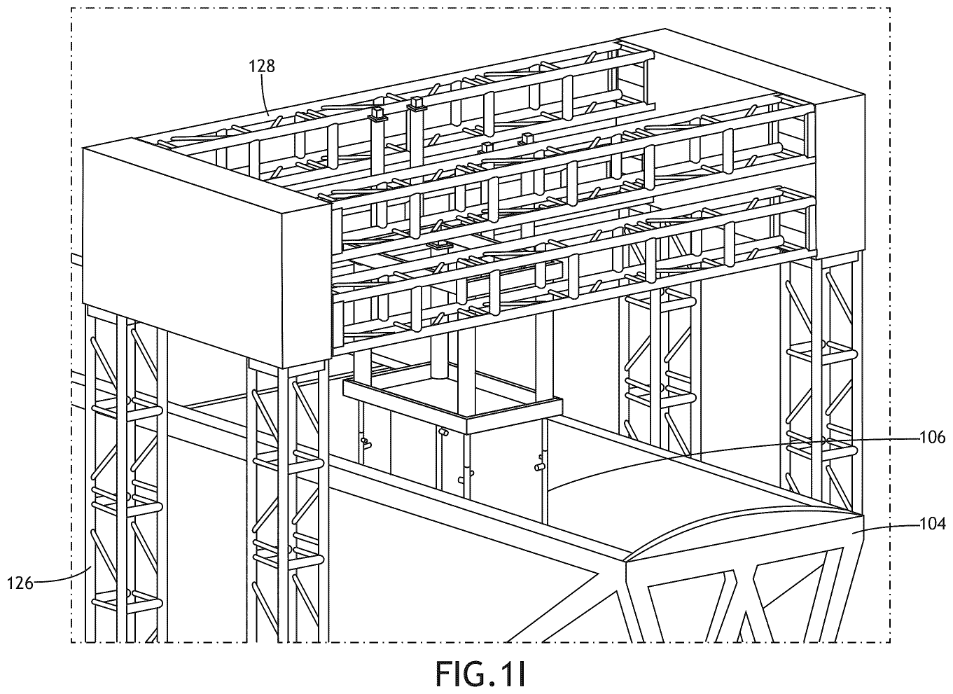

FIG. 1A illustrates a block diagram of a grading system 100, in accordance with one or more embodiments of the present disclosure. The grading system 100 may be configured to grade a material 102. The material 102 may be stored in a container 104. It is noted that while reference is made to material throughout the instant application, that material may include, and is not limited to, grains, fruits, vegetables, meat, livestock, or the like. The container 104 may include things such as, but not limited to, trucks, train cars, silos, flat storage, outdoor piles, conveyor belts, gantries, or other grain storage. It is noted that one or more grading systems 100 may be utilized for each container 104. It is further noted that the container 104 may continue to move while samples are taken by the grading system 100.

In embodiments, the grading system 100 includes one or more sampling probes 106. The sampling probes 106 may be configured to collect a sample of the material 102 and/or take one or more measurements of the material 102.

The sampling probes 106 may be pneumatic probes (e.g., telescoping vacuum probes designed to penetrate a portion of the material 102, withdraw a sample of the material 102, and retract prior to container 104 exit), a rotary core sampler (e.g., mechanized auger-based samplers capable of extracting a column of material 102), or a sweeper arm sampler (e.g., actuated arms with integrated scoops or conveyors to collect material 102 from the surface layer of the container 104).

The sampling probes 106 may travel in two dimensions, both forward and backward, as well as side-to-side across the dimensions of the container 104. The sampling probes 106 may also move in three dimensions, adding vertical movement to lift out of the container's way before and after taking a sample of the material 102. The sampling probes 106 may be powered by grid electrical power. The sampling probes 106 may be positioned above the container 104 and lowered when the container 104 is below and stationary. Alternatively, the sampling probes 106 may be located to the side of the container 104 and extend above it after stopping on the scale, or it may extend to the side of the container 104. In both cases, the goal may be to avoid interference with tall components like smokestacks, air diverters, or antennas by moving out of the way when not in use. It is noted that the sampling probes 106 may be capable of operating with a stationary container 104 or a moving container 104. Further, the sampling probes 106 may be suitable for handling multiple truckloads one after another.

It is noted that the sampling probes 106 may be uncovered or may require partial or full enclosure including one or more of a roof, walls, or moveable doors, pending the climate requirements of the customer site(s).

The sampling probes 106 may automatically determine a random sampling path that avoids obstructions and probe those random locations to withdraw one or more corresponding material 102 samples. The sampling probes 106 may measure the odor signature of the entire load of material 102, as well as each individual sample of material 102. The sampling probes 106 may scan the load of material 102 to identify the type of material 102 in each sample. The sampling probes 106 may also measure moisture levels, damage, foreign material content, test weight, and odor in each sample. Additionally, mycotoxin levels, oil content, protein content, and starch content may be measured. The graded material 102 data may be stored on-site, at a central server, or in the cloud for long-term storage. This may avoid retaining physical samples by keeping a digital record instead, which could include images, odor signatures, moisture content, foreign material levels, test weight, damage levels, and other relevant data.

In embodiments, the grading system 100 includes one or more sensors 108. The sensors 108 may include moisture sensors, odor sensors, optical sensors, volume and weight sensors to obtain test weight, hyperspectral or near infrared sensors, or the like. It is contemplated that the sensors 108 may be coupled to the sampling probes 106 such that the sensors 108 collect information of the material 102.

For example, optical sensors may collect optical data of the material 102. The optical data may include defects in the material 102, discoloration of the material 102, whiteness (e.g., for rice), insect damage, insects themselves, mold damage, heat damage, or foreign material within the material 102. The optical sensors may utilize computer vision. Computer vision may enable automated remote viewing of samples through captured images, where image analysis may be trained to categorize kernel size, estimate the number of kernels, detect foreign material, assess the percentage of damaged kernels, assess the amount of heat damage, identify presence of mold in the sample, and identify the possible presence of insects. An automated report may be generated to record these observations. Additionally, computer vision may be applied to estimate the percentage of foreign material, mold, cracks, broken kernels, and particle size in a sample or across the entire volume through either instantaneous snapshots or continuous scanning of the material 102 flow. It may also be used to assess the color of the material 102, which could help indicate potential disease or heat damage, or help to determine the type of material being measured, as some materials have similar size but differ due to color.

By way of another example, odor sensors may develop an odor signature for the material 102. The odor signature may indicate a type of the material 102, a spoilage of the material 102, the presence of undesirable materials such as fertilizer, drugs, explosives, fuels, treated seed, or one or more chemicals mixed with the material 102. The odor signature may be useful for detecting sour or musty smells which may indicate a material 102 is not of sufficient quality. In the case of fruits, the odor sensors might be trained to understand “sweetness” levels (e.g., a sweet smell in the fruits). In the case of vegetables, the odor sensors might be trained to understand vegetable-specific insects and to detect perishable levels. The odor sensors may include highly sensitive digital receptors grouped together as sensor array on a microchip. This may be able to detect millions of different gases and volatile organic compounds (VOC) combinations by collecting data and utilizing machine learning classification models to transform this data into insights as part of the grain grading system. This may enable the ability to detect and collect data characteristics specific to enable identifying grain irregularities caused by insects, disease, mold, or variations in crop type.

The moisture sensors may measure a moisture content of the material 102.

By systematically documenting and analyzing the odor profile upon the arrival of material 102 at commodity companies, more informed decisions may be made regarding grading, pricing, storage conditions, and marketability. Standardized procedures and quality control measures may help promote consistency and transparency in the trading process, potentially benefiting both producers and consumers across the agricultural supply chain.

The presence of a sour or musty smell in the dockage of material 102 may indicate microbial activity, mold growth, heat damage, or moisture damage within the sample. Dockage refers to foreign material such as dirt, stones, glass, chaff, insects, or broken particles that may be separated from the main lot during processing or cleaning. When dockage emits a sour or musty odor, it may suggest possible issues related to moisture, suboptimal storage conditions, or microbial contamination such as molds or fungi. The sour smell may result from organic acids produced during microbial decomposition, while the musty odor may be linked to mold growth and potential mycotoxin formation. These odors may serve as indicators of quality concerns that warrant further evaluation to avoid contamination of the primary supply. Addressing such odors may require prompt action to identify underlying causes, mitigate risks to quality, and prevent spoilage. Practices such as proper drying and aeration, maintaining ideal storage conditions through active monitoring and aeration automation, and conducting routine quality checks may be effective in protecting the integrity of material 102. Monitoring for odors associated with dockage may also help ensure high-quality outputs and support industry standards for safety, quality, and customer satisfaction.

Using the data collected from the sampling probes 106 and the sensors 108, grain deliveries, shipments, and grading may be simplified. For example, computer vision may be used to view a load or material 102 sample to help automatically determine which type of material 102 is in a truck, while odor sensing may be applied to analyze the smell of the load or sample for the same purpose. Similarly, computer vision may be used to view the contents of a bin or silo to identify the type of material 102, and odor sensing may assist in determining the material type based on scent. These technologies may support automated systems that direct trucks from the scale or sampling location to a corresponding bin or silo, potentially preventing the accidental mixing of material 102. Non-conforming material types may be flagged to help avoid dumping errors, and automated warning systems may inform site managers of potential incorrect dumping events, such as identification of treated seed, fertilizer, fuel, or other nonconforming material that cannot be accepted by a receiving site. Additionally, automated doors may be programmed to open only when a confirmed match is made between the material 102 in the truck and the material 102 in the assigned dump pit, bin, flat storage structure, pile, or silo. A system of lights at the dumping site may also guide trucks to the correct dumping location based on automated identification and matching processes. It is further contemplated that the containers 104 may be embodied as autonomous trucks. In such a case, the autonomous trucks may receive signals, where the signals guide the autonomous trucks to a dumping site or notify whether or not a load has been accepted.

In embodiments, the grading system 100 includes a support structure 110. It is noted that the support structure 110 may be deployed as a boom arm support structure 110a (e.g., as shown in FIG. 1B) or a gantry support structure 110b (e.g., as shown in FIGS. 1C-11). Broadly speaking, the support structure 110 may be any structure that is capable of positioning the sampling probes 106 above the container 104.

In embodiments, the grading system 100 includes one or more motors 112. The motors 112 may be one or more of electric motors, hydraulic motors, hydraulic cylinders, electric actuators, or any other device that is capable of actuating the sampling probes 106. Broadly speaking, the motors 112 may be any component capable of causing actuation of the sampling probes 106.

In embodiments, the grading system 100 includes a controller 114 communicatively coupled to any components therein. In embodiments, the controller 114 includes one or more processors 116. For example, the one or more processors 116 may be configured to execute a set of program instructions maintained in a memory 118. For example, the program instructions may be configured to cause the processors 116 to execute one or more steps of the present disclosure. In embodiments, the grading system 100 includes a user interface 120 communicatively coupled to the controller 114.

For example, the program instructions may cause the processors 116 to actuate the sampling probes 106 such that the sampling probes 106 are inserted into the container 104 at one or more locations to collect the information of the material 102 within the container 104. By way of another example, the program instructions may cause the processors 116 to receive the information collected by the sensors 108. By way of another example, the program instructions may cause the processors 116 to grade the material 102 within the container 104 based on the information collected by sensors 108.

The processors 116 may also be configured to generate alerts if something is not right with the material 102. For example, the processors 116 may determine if the odor signature for the material 102 is within selected bounds and provide an alert if the odor signature is not within the selected bounds. By way of another example, the processors 116 may determine, based on the optical data, if the material 102 is a correct type of material 102 and provide an alert if the type of material 102 is incorrect. By way of another example, the processors 116 may determine, based on at least one of defects in the material 102, discoloration of the material 102, or foreign material within the material, if the optical data for the material is within selected bounds and provide an alert if the optical data is not within the selected bounds. By way of another example, the processors 116 may determine if the container 104 is located in a correct area and provide an alert if the container 104 is located in an incorrect area. By way of another example, the processors 116 may determine if the odor of the material in the container 104 contains traces of non-conforming materials such as fertilizer, treated seed, or fuel, and provide an alert if the container 104 has traces of any of these non-conforming materials.

In embodiments, the controller 114 may be coupled to the sampling probes 106, the sensors 108, the motors 112, or any other component of the grading system 100.

In embodiments, one or more identifiers 122 are located on the container 104. It is noted that the identifiers may be utilized in order to track the material 102 through the processes of sourcing, receiving, grading, and shipping, as well as other practices.

The identifiers 122 may be any type of identifier that allows traceability of the material 102 (e.g., through detection by the sensors 108). For example, the identifiers 122 may be a license plate, a quick response (QR) code, a universal product code (UPC), a radio-frequency identification (RFID) tag, a global navigation satellite system (GNSS) tracker, or a global positioning system (GPS) tracker.

Knowing the specific location where a load of material 102 originated may offer several important benefits for both producers and consumers. It can enhance traceability by enabling precise tracking of material 102 from source to end, which is vital for food safety and regulatory compliance. Such traceability may help identify and isolate issues like contamination or quality defects to particular fields, allowing for quicker corrective actions. It may also support more accurate and targeted agronomic decisions, as farmers could analyze field performance and implement tailored management practices to improve yield and quality. Additionally, field-specific data may provide valuable insights into soil health, pest pressures, and weather impacts, encouraging more sustainable farming practices. For consumers, this level of transparency may increase trust and confidence in the food supply chain. Overall, field-level traceability may improve quality control, sustainability, and consumer assurance.

Leveraging vehicle-based GPS trackers, it may be possible to automatically associate a load of material 102 with a graded grain sample, a combine harvester, a field, a farm, and the grower. This may allow linking the sampled material 102 to one or more parameters in order to proactively adjust harvesting activities and maximize potential revenue. For example, it may be valuable to inform a combine harvester driver to move to a drier part of the field if trucks are delivering grain with excessively high moisture content. Similarly, drivers may be advised to adjust combine settings if the harvested grain shows signs of excessive damage or contains too much foreign material.

Digitally recording specific loads of materials 102 along with their parameters and track where those material 102 are stored within grain storage facilities may be performed. Odor signatures from grain entering the storage structure may be linked to those detected during unloading, automatically associating the grain with its field of origin. Additionally, scale weight data may be automatically captured and assigned to specific loads, further enhancing traceability and quality control.

In embodiments, an RFID chip may be used as the identifier 122. Each bag or load of material 102 may be tagged with an RFID chip containing information about the field of origin, date of harvest, and crop details. As the material 102 is loaded into containers 104, RFID readers at the loading point may scan the tags, logging the origin and details of each load. Upon delivery to the facility, RFID readers at the entry gate may scan the container's contents, update delivery details and linking them to the originating field. The container 104 may be weighed on a digital scale that records the total weight and associates it with the truck's RFID data, providing accurate weight information for each field's delivery. During unloading, RFID readers on conveyors and augers may continue to track the movement of material 102, ensuring that the origin is logged at every stage. Finally, material 102 may be stored in bins, flat storage, piles, or silos equipped with level sensors that monitor volume and link the data back to the field of origin, maintaining traceability throughout storage.

In embodiments, a GPS tracker may be used as the identifier 122. Material 102 may be harvested and loaded directly into containers 104 equipped with GPS trackers that log the exact location of each load's origin. A digital scale-enabled cart at the field may weigh the material 102 before it is loaded into the container 104, recording the weight and linking it to the GPS data due to proximity of containers 104 during loading and/or unloading operations. Containers 104 with GPS capabilities may continuously transmit their location and status to a central system, providing real-time tracking from the field to the facility, or alternatively, may record their location and upload the data when in proximity to a data uplink for non-real-time tracking. Upon arrival at the facility, containers 104 may be weighed again to verify the load weight, with the digital gross and tare scale data combined with GPS information to confirm the field of origin. Automated conveyors and augers at the facility may be equipped with barcode or QR code readers that scan tags attached to the material 102 loads, further confirming their origin. Sensors 108 may monitor material 102 volume, and track the specific field source of each batch, ensuring complete traceability throughout storage.

embodiments, a blockchain-integrated system may be used as the identifier 122. During harvesting, data about the field, such as crop type, pesticides used, planting date, harvest date, water applied, cropping operations, specific seed hybrid, and fertilizer application, may be uploaded to a blockchain ledger via mobile devices or internet of things (IoT) devices. Containers 104 equipped with loT devices may record and transmit loading data, including weight and GPS coordinates, to the blockchain ledger. At the facility's weigh station, scales integrated with the blockchain system may automatically record the weight and verify it against the initial load data. As material 102 is unloaded, loT devices on conveyors and augers may track and record the unloading process on the blockchain ledger, ensuring that each batch's movement is documented. Bins and silos with loT devices may continuously monitor and record material 102 volume and storage conditions, such as temperature and humidity, on the blockchain, maintaining a trace of the journey of the material 102 from field to storage.

Referring now to FIG. 1B, aspects of the boom arm support structure 110a are discussed in further detail. In embodiments, the boom arm support structure 110a includes one or more arms 124. The arms 124 may be coupled to one another. It is contemplated that one arm 124 may be secured to the ground and stationary, while another arm 124 is actuated. Motors 112 may rotate and move the sampling probes 106 into the grain container 104 to access the material 102. The sampling probes 106 may be located at an end of an arm 124.

Referring now to FIGS. 1C and 1D, aspects of the gantry support structure 110b are discussed in further detail. In embodiments, the gantry support structure 110b includes one or more vertical members 126 and one or more horizontal members 128. Pairs of the vertical members 126 may be located on opposite sides of the container 104 from each other. The horizontal members 128 may be coupled to a pair of the vertical members 126, where the horizontal members 126 span the container 104. The sampling probes 106 may be coupled to the horizontal members 126, with the motors 112 providing motion for the sampling probes 106 in at least two dimensions along the horizontal member 126. It is further contemplated that a single vertical member 126 may be used such that the gantry support structure 110b is located on the side of the container 102.

It is noted that the gantry support structure 110b may be static (e.g., as shown in FIG. 1C), such that the container 104 is positioned under it, or mobile (e.g., as shown in FIG. 1D) such that the gantry support structure 110b is positioned over the container. In FIG. 1C, this 1C configuration, the sampling probes 106 may be activated vertically, or along an arc, to descend into the container 104.

FIGS. 1E and 1F illustrate a grading system 100 with more than one sampling probe 106, where the sampling probes 106 are in a stored state, in accordance with one or more embodiments of the present disclosure. FIGS. 1G-11 illustrate a grading system 100 with more than one sampling probe 106, where the sampling probes 106 are in a deployed state, in accordance with one or more embodiments of the present disclosure. For example, when the grading proves are in a stored state, it may be easier to position a container 104 under the sampling probes 106. However, it may be necessary to deploy the sampling probes 106 before sampling the material 102. The sampling probes 106 may be moved between the deployed and stored position with motors 112.

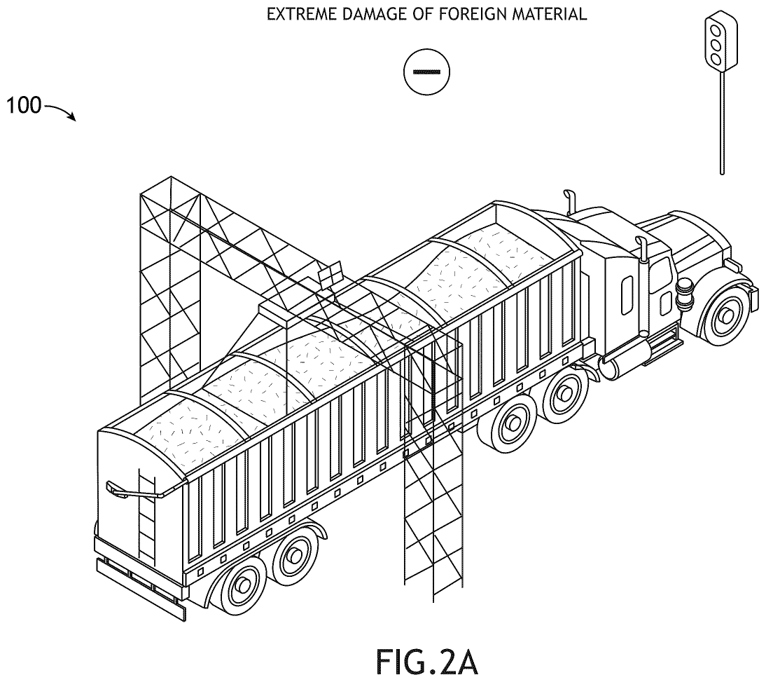

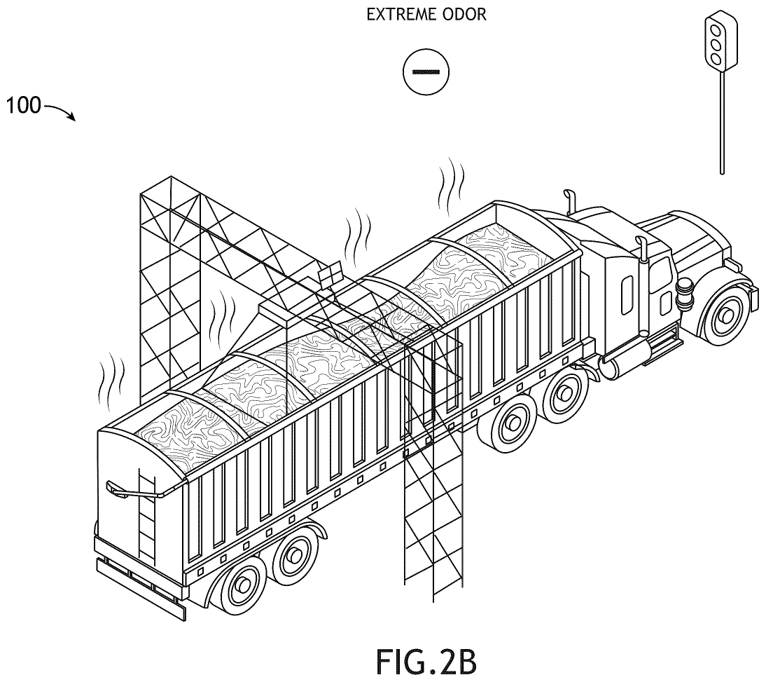

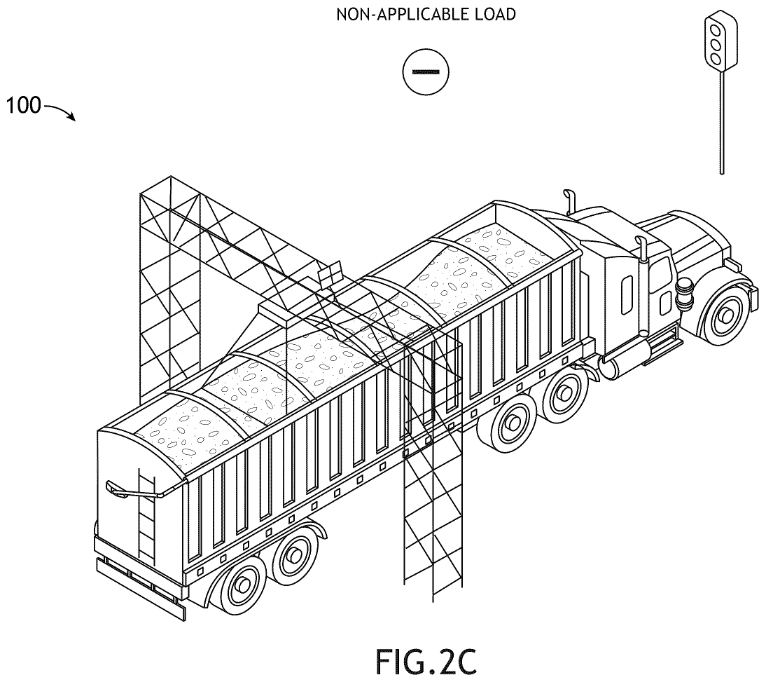

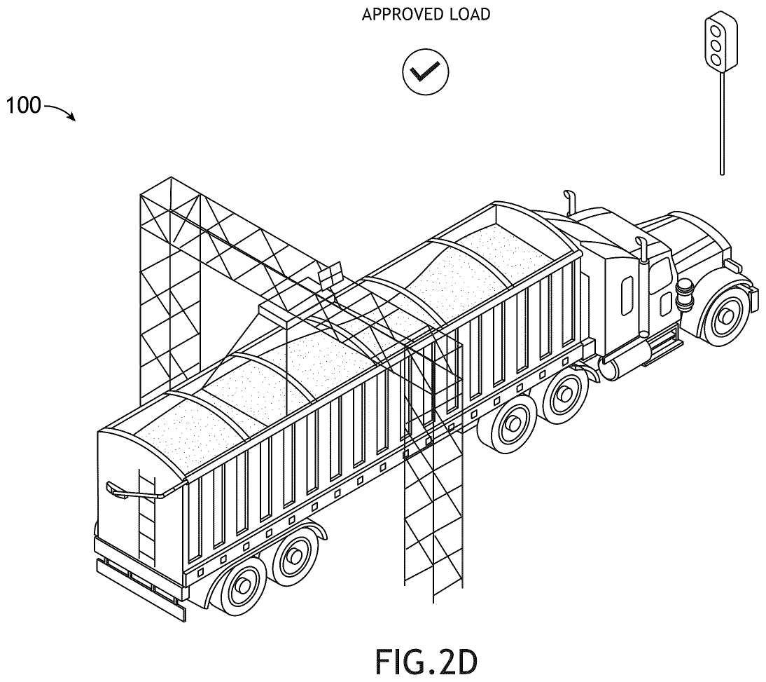

FIG. 2A illustrates rejection of material 102 due to damage, in accordance with one or more embodiments of the present disclosure. FIG. 2B illustrates rejection of material 102 due to odor, in accordance with one or more embodiments of the present disclosure. FIG. 2C illustrates rejection of material 102 due to the material not being correct, in accordance with one or more embodiments of the present disclosure. FIG. 2D illustrates approval of material 102 due to criteria being met, in accordance with one or more embodiments of the present disclosure. It is noted that FIGS. 2A-2D illustrate a step in the process of receiving material 102 that allows the receiving company to reject the truck before dumping, even prior to the sampling probe 106 being inserted into the material 102.

The grading system 100 may be powered on and undergo a self-check to ensure all components are functioning properly. The sensors 108 may be activated to monitor incoming containers 104. As a container 104 approaches, the system may capture optical data from multiple angles to support accurate material identification. Data container 104 identifiers 122, such as geospatial systems, RFID tags, or license plates, may be integrated into the grading system 100 to help confirm a unique container identifier. Advanced image recognition algorithms may then analyze the captured optical data against a pre-existing database of registered container identifiers or license plates to verify identity. Once the container 104 is successfully identified, the grading system 100 may confirm its registration status and notify the central management system of its arrival.

It is noted that the sampling probes 106 may be used to automatically gather a series of material 102 samples from the container 104 in a random manner to minimize fraud. This may allow for the grading system 100 to probe the container 104 in completely random ways and capture samples of material 102 from all depths and across the full topmost area within the container 104, reducing the chance that incoming material 102 has issues, such as excess moisture, foreign material, or non-conforming materials such as fertilizer, fuel, treated seed, rocks, or the like. The sampling probes 106 may extend into the material 102 using the motors 112, penetrating the material 102 to collect samples without disrupting the load significantly. It is noted that the sampling probes 106 may also be programmed to enter the container 104 in pre-programmed locations.

The graded sample may be retained without needing to move the material 102 to a scale-house. When the container 104 stops on the scale, the grading system 100 may use computer vision to scan the container 104 and automatically calculate any obstructions such as cross beams. However, it should not be considered limiting for the grading system 100 to be located at the scale, as many newer receiving facilities separate the scaling from the grain sampling, but some locations still have both operations at the same physical location.

Before sampling with the sampling probes 106, the sensors 108 (e.g., the optical sensors) may scan the container 104 to determine if there are any obstructions. For example, the grading system 100 may also determine whether the container tarp is rolled back. If it is not, the system may avoid taking a sample of the material 102 and automatically inform the operator of the error. If the tarp is rolled back, the grading system 100 may proceed to take one or more material 102 samples. The grading system 100 may use guided technology to make real-time adjustments based on the orientation and movements of the container 104, ensuring precise alignment, and avoiding damage to cross members and other container components. The grading system 100 may use pressure or force sensing to provide numerical inputs to ensure that the one or more sampling probes 106 do not push so hard that it breaks through the bottom of the container 104 or damage customer infrastructure. The sampling probes 106 may extract material 102 samples from different strata of the container 104 to obtain a representative sample of the overall material quality. If necessary, the sampling probes 106 may perform multiple insertions at different locations within the container 104 to ensure comprehensive sampling. This frequency and number of samples may be set by the material receiving company (e.g., a grain company who is receiving the grain). Additionally, the sampling probes 106 may be configured to remove more than one sample of the material 102 from the container 104 each time the sampling probes 106 are inserted into the container 104, wherein each sample of the more than one samples is collected from a different location within the container 104.

After collection, samples of material 102 may be graded (e.g., by the processors 116 in cooperation with the sensors 108). This may include evaluating foreign material content, identifying cracks, testing moisture levels, assessing bulk density, detecting odors, and measuring other relevant quality metrics. Once analysis is complete, the results may be automatically compiled and reported. The grading data may then be digitally linked to the scale ticket, used to alert relevant personnel if any quality issues are detected, and update the inventory management system accordingly. If certain preset criteria are met, such as the presence of treated seed, fuel, fertilizer, mycotoxins, explosives, excessive foreign material, or identification of mixed material, the grading system 100 may automatically reject the load. Similarly, if other thresholds are exceeded, such as high moisture content or foreign material, the system may assign a docked price to the load based on those conditions.

The grading system 100 may process feedback from previous operations to refine its algorithms and improve accuracy for future identifications and samplings. After completing a cycle, the sampling probes 106 may reset and prepare for the arrival of the next container 104.

Various measures may be taken in order to increase the level of human error possible, in order to maintain the accuracy and efficiency of the grading system 100. For example, a traffic light or another notification system may be incorporated to inform an operator or an autonomous container that they are permitted to leave the sampling area and proceed to the designated dumping location once the grading process is complete. This traffic signal may also be integrated into the on-site routing system to guide the container 104 to the correct dumping location after scaling, sampling, and grading are finished. Additionally, an automated gate or door may be included in the route to prevent the wrong container 104 from dumping into the incorrect pit or material 102 conveyance system. Automated gates and/or conveyor interlocks may further be implemented to ensure that only the appropriate container 104 unloads into the designated pit or dumping infrastructure at the receiving facility. This concept of fool-proofing (e.g., preventing unwanted operations to occur) can also be applied to intrasite material conveyance operations, as well as outgoing material operations, often using trucks and/or train cars for movement of the loaded material.

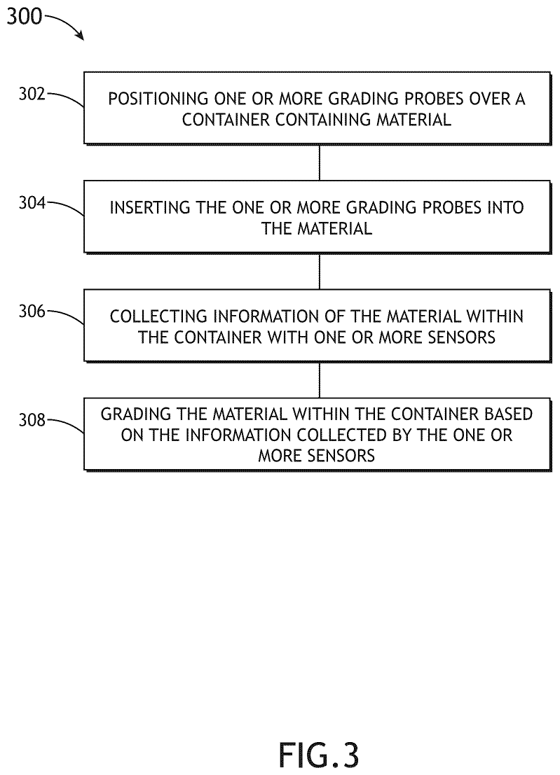

FIG. 3 illustrates a flow diagram of a grading method 300, in accordance with one or more embodiments of the present disclosure. Applicant notes that the embodiments and enabling technologies described previously herein in the context of the grading system 100 should be interpreted to extend to grading method 300. It is further noted, however, that the grading method 300 is not limited to the architecture of the grading system 100.

In embodiments, the grading method 300 includes a step 302 of positioning one or more sampling probes over a container containing a material. For example, the sampling probes may be positioned over the container by a support structure. The support structure may be designed such that it sits above or to the side of the container and elevates the sampling probes above the container.

In embodiments, the grading method 300 includes a step 304 of inserting the one or more sampling probes into the material. For example, the sampling probes may be actuated by one or more motors (e.g., electric motors, electric actuators, or hydraulic cylinders, or the like), such that they are inserted into the material. The sampling probes may be inserted into random locations of the material in order to get an accurate representation of the qualities of the material. Additionally, the sampling probes may avoid obstacles on the container, such as cross supports or coverings when being inserted into the material after the container is scanned by the sensors. The sampling probes may also be configured to remove more than one sample of the material from the container each time the sampling probes are inserted into the container, wherein each sample of the more than one samples is collected from a different location within the container.

In embodiments, the grading method 300 includes a step 306 of collecting information of the material within the container with one or more sensors after the one or more sampling probes deliver the material to the one or more sensors. The sensors may include one or more odor sensors to detect one or more odor signatures of the material, one or more optical sensors to collect optical data of the material, and/or one or more moisture sensors to measure a moisture content of the material. The odor signatures of the material may include the ability to automatically detect a specific material, a spoilage of the material, or the presence of one or more chemical mixed with the material. The optical data may include one or more images of the material, defects in the material, discoloration of the material, and/or foreign material within the material.

In embodiments, the grading method 300 includes a step 308 of grading the material within the container based on the information collected by the one or more sensors. For example, the material may be graded based on any combination of the odor signature, the optical data, or the moisture content.

In embodiments, the grading method 300 includes a step of determining if one or more odor signatures for the material is within selected bounds and providing one or more alerts if the odor signature is not within the selected bounds. For example, if the odor signature indicates spoilage, an incorrect material, or chemicals, an alert may be provided.

In embodiments, the grading method 300 includes a step of determining, based on the optical data, if the material is a correct type of material and providing an alert if the type of material is incorrect.

In embodiments, the grading method 300 includes a step of determining, based on at least one of the defects in the material, the discoloration of the material, or the foreign material within the material, if the optical data for the material is within selected bounds and providing an alert if the optical data is not within the selected bounds For example, if there are too many defects, if the material is discolored beyond an acceptable level, or if there is too much foreign material present, an alert may be issued, which may lead to the container being rejected, docked, or segregated from other received materials.

In embodiments, the grading method 300 includes a step of identifying one or more identifiers on the container. The container may include identifiers which may indicate a location the material is from, a type of material, or the like.

In embodiments, the grading method 300 includes a step of determining if the container is located in a correct area and providing an alert if the container is located in an incorrect area. For example, if the indicators show that the container is located in the wrong area for that type of material, and alert may be issued. Furthermore, if the container is located in certain unauthorized areas, gat, conveyor, or other lock-outs may be engaged to prevent contamination of materials.

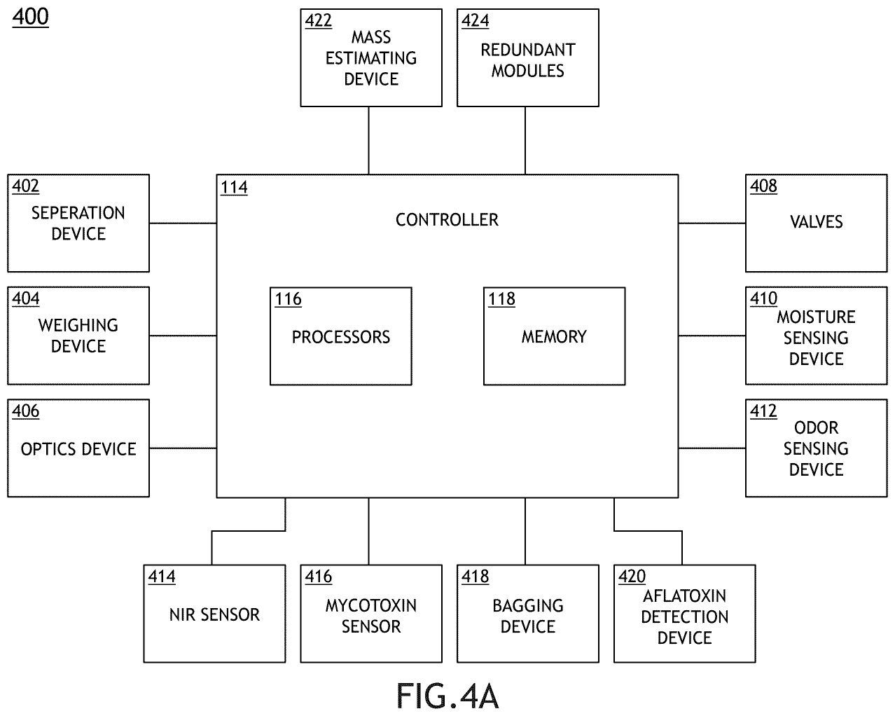

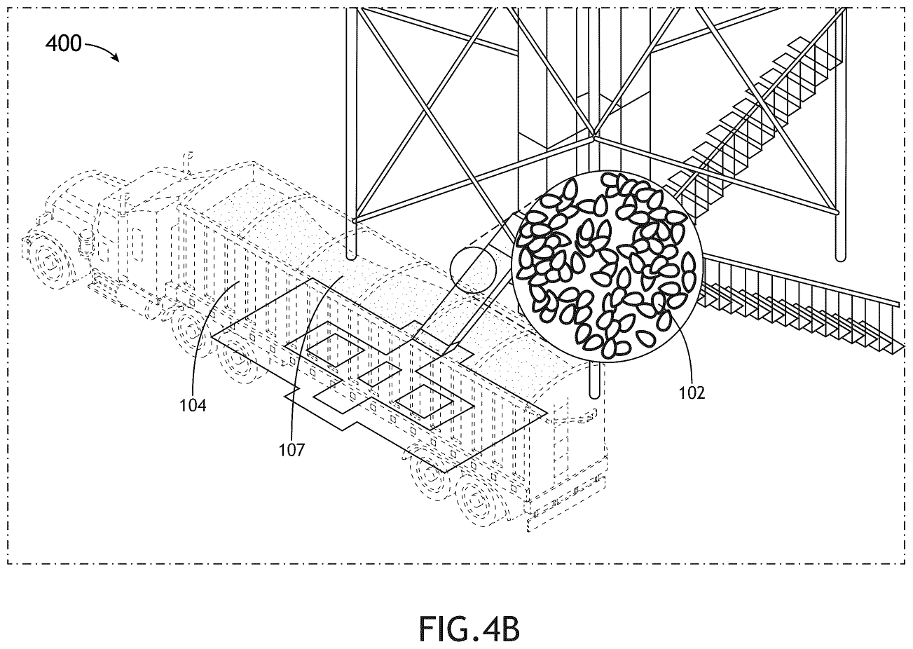

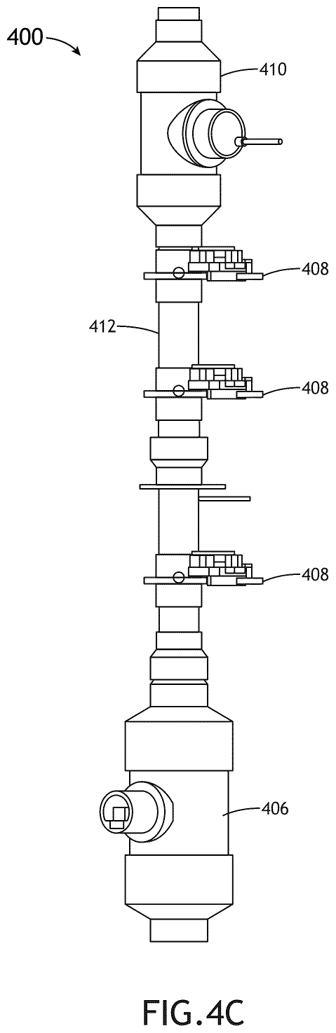

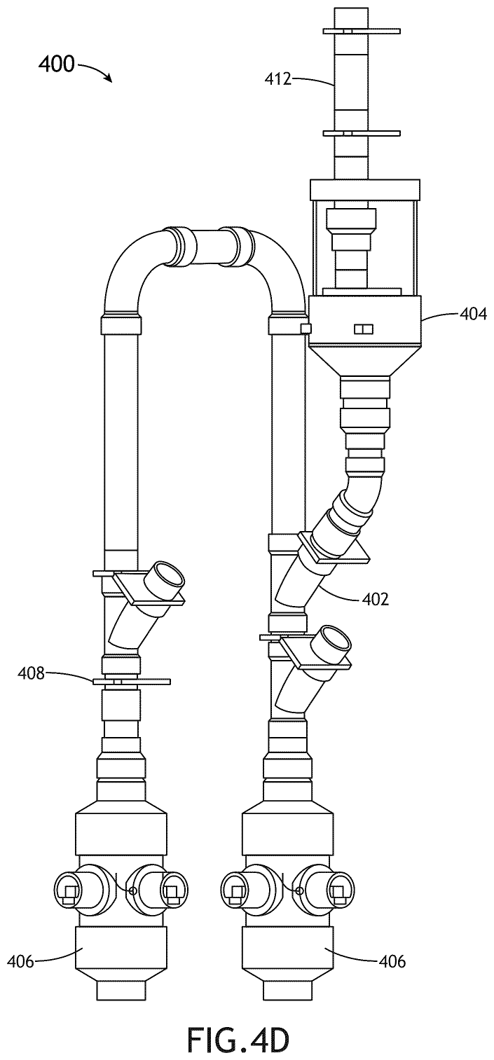

FIG. 4A illustrates a block diagram of a continuous grading system 400, in accordance with one or more embodiments of the present disclosure. FIGS. 4B and 4C illustrate a continuous grading system 400, in accordance with one or more embodiments of the present disclosure. It should be noted than any aspects (e.g., modules or devices) of the continuous grading system 400 described herein may be modular, in that a particular device or module may be removed from the continuous grading system 400, while retaining the functionality of all other modules in the continuous grading system 400. Additionally, the continuous grading system 400 may be continuous. In this way, material 102 may flow from one module to another without the need for benchtop instruments or human intervention (e.g., to load and/or unload the material 102). It is further noted, that due to the modularity of the continuous grading system 400, any components may be positioned in any order, while retaining the desired effect. Further, any components of the continuous grading system 400 may be in line (e.g., in series) with each other such that the material 102 passes from one component to another without the need for human, or any other external, intervention.

The continuous grading system 400 may also receive material 102 from a flow of material 102. After it passes through the continuous grading system 400 and been graded, it may be returned to the flow of the material 102.

It is noted any aspect and/or functionality of the grading system 100, including, but not limited to, the sampling probes 106, the support structure 110, and the motors 112 may be applicable to the continuous grading system 400 (e.g., to introduce material 102 to the continuous grading system). However, it should be noted that the material 102 does not need to be introduced to the continuous grading system 400 with the sampling probes 106, the support structure 110, and the motors 112, and may be introduced to the continuous grading system 400 in any manner. For example, the continuous grading system 400 may be attached to the sampling probes 106 outside the traditional scale house or kept in the traditional scale house using the traditional pneumatic system for moving the samples of the material 102. In this way, the material 106 mat enter the continuous grading system 400 from a container 104 by a sampling probe 106, the continuous grading system 400 may grade the material, and return the material 102 the container 104 with the grading probe 106, after the material 102 has been graded. In this way, the material 102 may move from the sampling probes 106 (e.g., be removed from the container 104 by the sampling probes 106) to the continuous grading system 400 and back to the container 104 without the need for human, or any external, intervention.

It is further noted that the material 102 may automatically be blended based on the grade of the material 102 such that the blending achieves target specifications. For example, automatically blending the material 102 using data outputs from the continuous grading system 400 to adjust gates and grain handling equipment to fine tune the blending process to keep the material 102 within target specifications may be possible based on the grading of the continuous grading system 400.

The continuous grading system 400 may be integrated with one or more cameras and lighting spectrums designed to detect specific characteristics of material 102, such as material not matching the intended product, breaks or cracks in the material 102, presence of mold, presence of heat damage, presence of germ damage, level of protein content, level of oil content, starch content, and indication of mycotoxins, or the like. Additionally, the continuous grading system 400 may incorporate odor sensors tuned to identify specific VOC signatures, which in the context of the material 102 may be analogous to how human smell is used in the grain industry to detect sour or musty odors. The continuous grading system 400 may be mounted onto existing handling equipment and may house sensors, cameras, lighting, and odor-sensing components. Grading data gathered by the continuous grading system 400 may be transmitted to downstream handling equipment, enabling automated routing of the container 104 based on the grading results. The odor sensors may be trained to identify a specific odor signature or odor profile, including target odors (e.g., odors that may pertain to characteristics of the material 102) in the presence of background odors.

Given the variability in quality across different loads, there may be strong interest in equipment capable of continuously grading the full volume of material 102 as it is dumped. To enable this, it is envisioned that a structural addition be installed at existing dumping pits at facilities. This structure may include one or more ramps to elevate containers 104 above the current infrastructure, creating sufficient space beneath to house sensors that assess either a portion or the full stream of material 102 as it is discharged.

In the continuous grading system 400, a representative stream of material 102 or all of the material 102 may move through an environment that continually measures various quality parameters such as moisture content, foreign material, heat damage, mold, cracks, odor, protein content, oil content, starch content, mycotoxin levels, or the like. When continuous grading data is available, it may be possible to automate gates connected to containers 104, allowing the desired material 102 to pass, while blocking other material 102.

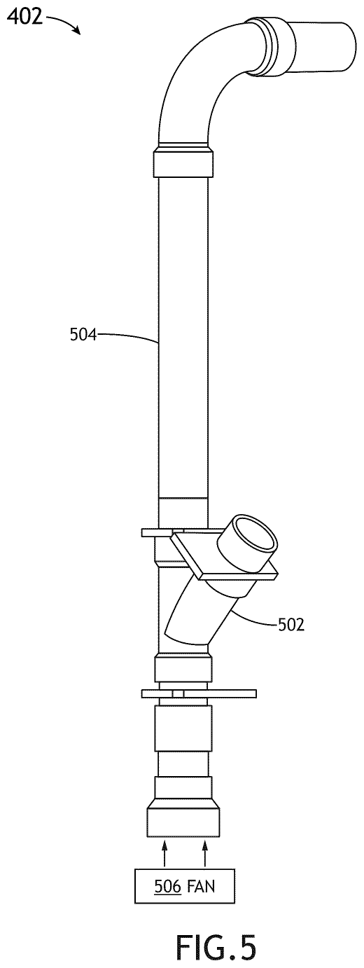

In embodiments, the continuous grading system 400 includes a separation device 402. The separation device may separate foreign material from the material 102 with a gas at selected velocity. Referring now to FIG. 5, the separation device 402 is discussed in greater detail. FIG. 5 illustrates the separation device 402, in accordance with one or more embodiments of the present disclosure. It is conceived that the velocity of the gas will be different depending on the materials being separated. For example, the separation velocity of gas for use in corn is different than the separation velocity for use in soybeans.

In embodiments, the separation device 402 includes a first tube 502.

In embodiments, the separation device 402 includes a second tube 504. The first tube 502 and the second tube 504 may be coupled to each other such that the second tube 504 enters the first tube 502 at an angle (e.g., an acute angle) relative to the first tube 502. The second tube 504 may introduce a combination of foreign material and the material 102 to the first tube 502.

In embodiments, the separation device 402 includes fan 506. The fan 506 may blow a gas at a selected velocity into the first tube 502. The gas may separate the material 102 from the foreign material and direct the foreign material in one direction in the first tube 502, while the material 102 falls in a second direction of the first tube 502. It is noted that the selected velocity of the gas may be dependent on the material 102.

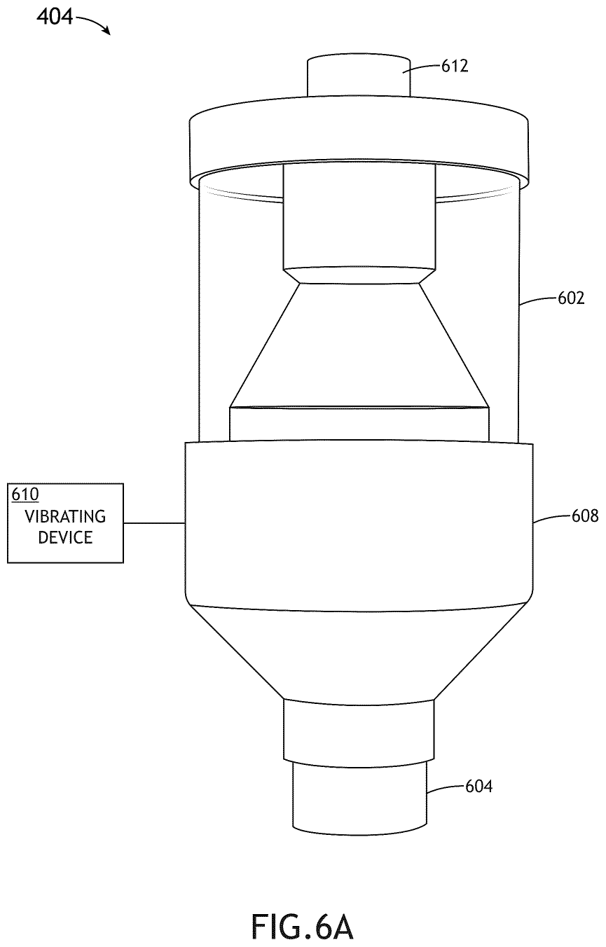

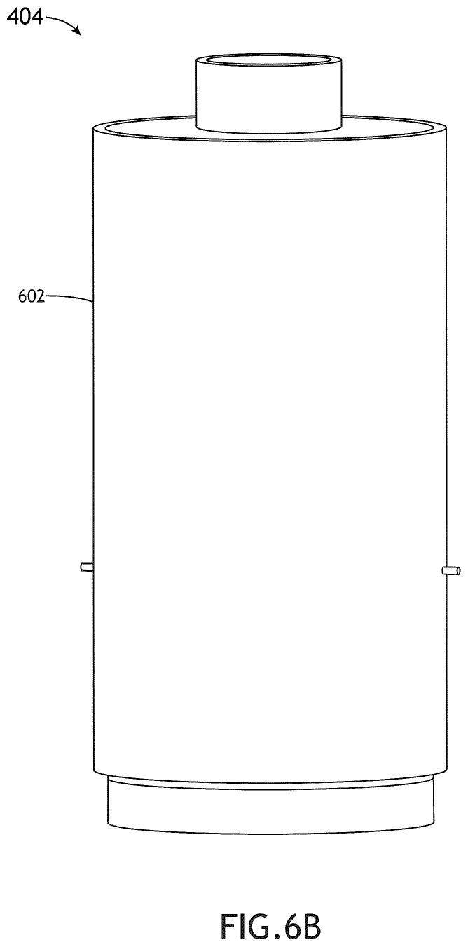

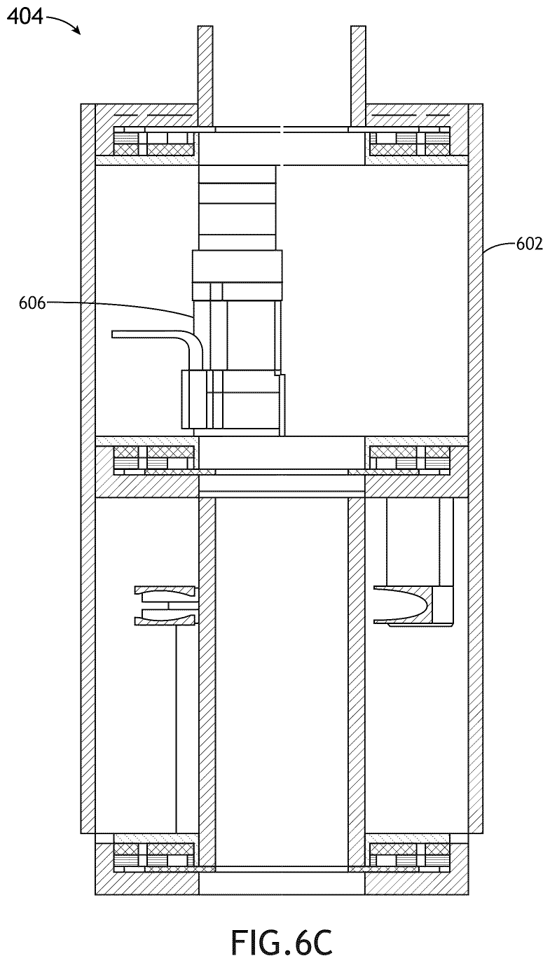

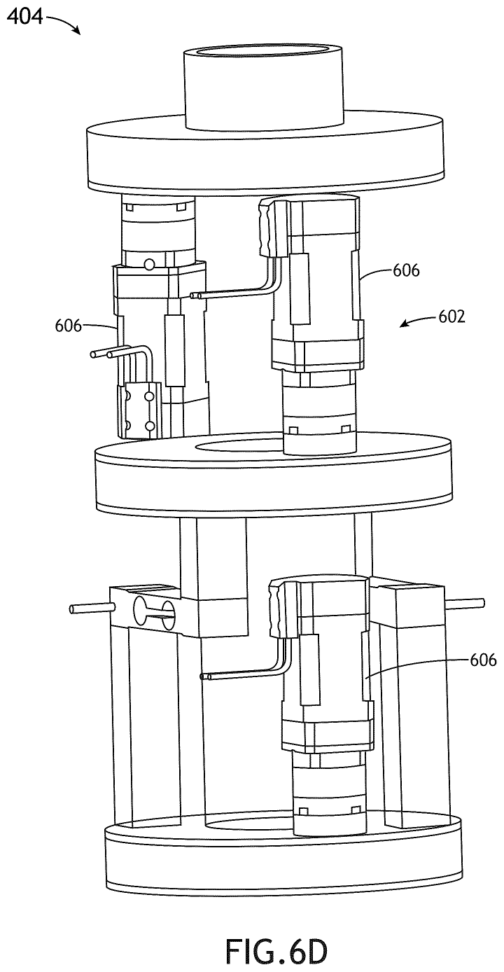

In embodiments, the continuous grading system 400 includes a weighing device 404. The weighing device 404 may weigh the material 102 that has been separated from foreign material. Referring now to FIGS. 6A-6D, the weighing device 404 is discussed in greater detail. FIGS. 6A-6D illustrate the weighing device 404, in accordance with one or more embodiments of the present disclosure. It is noted that the weighing device 404 may be used to determine test weight, weight, or bulk density.

In embodiments the weighing device 404 includes a fixed volume 602. A fixed volume 602 (e.g., a container with a known volume) may be positioned in-line with a pressurized tube system that conveys falling material 102 to the weighing device 404. As the material 102 flows into the weighing device 404, it may fill the fixed volume 602.

In embodiments the weighing device 404 includes a bottom valve 604. The bottom valve 604 may remain closed while the fixed volume 602 is being filled to prevent the material 102 from exiting. The weighing device may also include a top valve that closes upon being filled to cut off any excessive volume, prior to the volume being weighed.

In embodiments the weighing device 404 includes one or more load cells 606. Load cells 606 may be attached to the fixed volume 602 and may be used to weigh the material 102, automatically capturing a digital weight that is stored and linked to the specific sample of material 102. Before the material 102 enters the fixed volume 602, the load cells 606 may be tared to ensure accurate measurement. Since the volume of the fixed volume 602 is already known, the tared weight may be applied to this volume to calculate a digital density for each sample of material 102. Once the weight measurement is complete, the bottom valve 604 of the weighing chamber may be activated, allowing the material 102 in the fixed volume 602 to be released and combined with the remaining portion of the material 102 for further analysis. Meanwhile the next sample may already be queued up against the top valve to be ready to fill the volume chamber upon exit of the previous sample material.

In embodiments the weighing device 404 includes a lower container 608. The lower container 608 may be positioned below the fixed volume 602.

In embodiments the weighing device 404 includes a vibrating device 610. As material 102 is conveyed to the weighing device 404, it may fill the fixed volume 602 until it begins to overflow. The excess portion of the material 102 may fall around the fixed volume 602 and collect in the lower container 608. Once the material 102 has fully flowed and the fixed volume 602 is overflowing, the vibrating device 610 may be activated to level the surface of the material 102 within the fixed volume 602 (e.g., by shaking the fixed volume 602). This vibration may continue until the surface is uniform and flat, helping to ensure a consistent and accurate measurement of weight and/or bulk density without compacting the material 102.

In embodiments the weighing device 404 includes an upper valve 612. Once the container is filled and begins to overflow, an upper valve 612 may close to seal the material 102 inside the fixed volume 602. It is important that this upper valve 612 does not compress the material 102 within the fixed volume 602, as any compression could alter the weight and/or bulk density measurements.

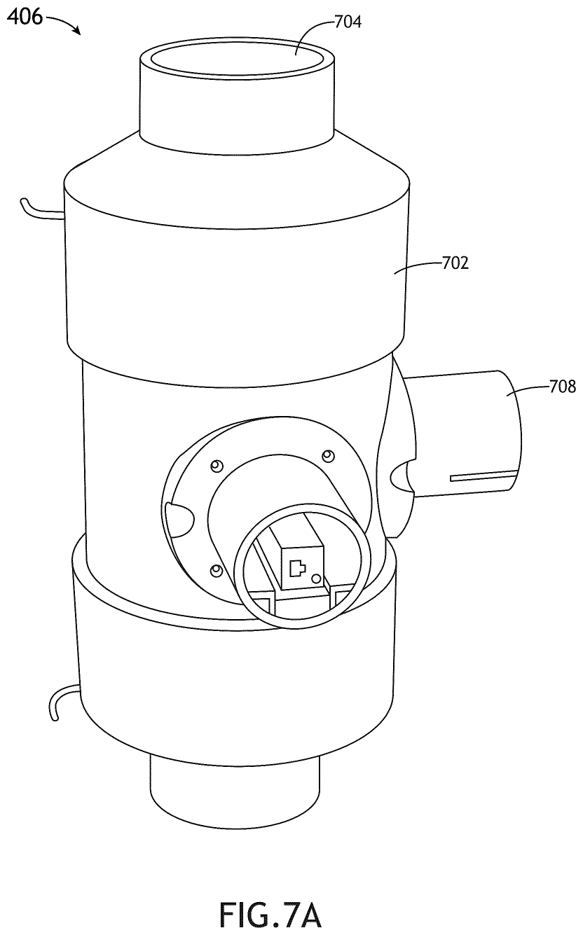

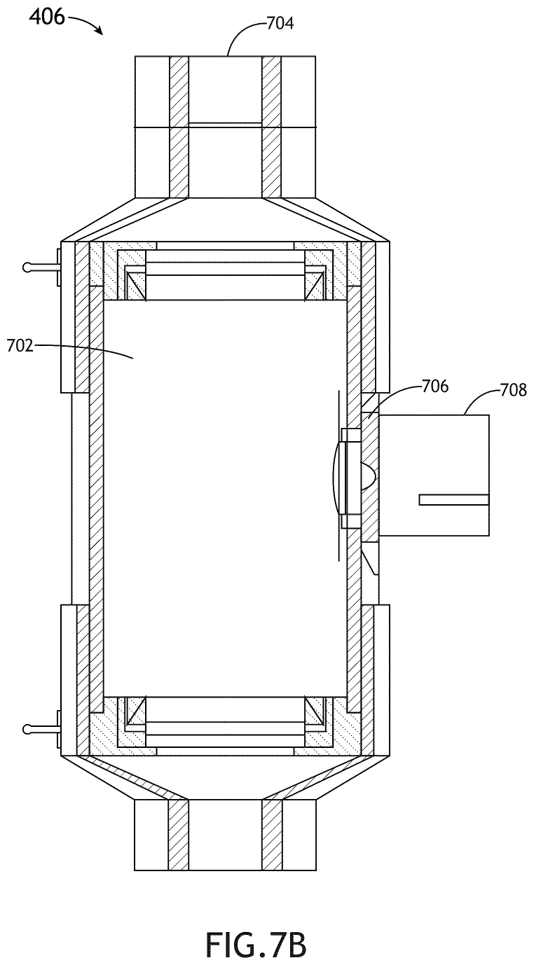

In embodiments, the continuous grading system 400 includes an optics device 406. The optics device 406 may be configured to capture one or more images of the material 102 that has been separated from the foreign material as the material 102 passes through the optics device 406. The optics device 406 may image the material 102 during freefall. Referring now to FIGS. 7A and 7B, the optics device 406 is discussed in greater detail. FIGS. 7A and 7B illustrate the optics device 406, in accordance with one or more embodiments of the present disclosure.

The optics device 406 may determine if the one or more images of the material 102 indicates the material is an incorrect material. If the material 102 is incorrect, the continuous grading system 400 may prevent mixing with the correct material 102.

In embodiments, the optics device 406 includes a chamber 702. The chamber 702 may be designed to allow the material 102 to fall through the chamber 702 without obstruction. In this way, the material 102 may fall through the chamber 702 as a steady stream. The interior of the picture chamber 702 may be painted with a light-reflective color to help ensure full illumination when images of material 102 are captured. Alternatively, the background might have a contrasting color to ease picking out specific targets from the resulting images using one or more algorithms or other artificial intelligence tools.

In embodiments, the optics device 406 includes a valve 704. The valve 704 may be configured to control the amount of material 102 to be imaged by opening to a selected amount (e.g., based on the type of material 102 being imaged). In this way, the valve 704 may control the stream of material 102 through the optics device 406.

The valve 704 may be designed to alter the path of the material 102 through the chamber 702. For example, the material 102 may be controlled such that it predominantly aligned perpendicular to the imaging axis and falls straight through the chamber 702. This orientation may maximize the accuracy of a length measurement in a single image, improving the confidence in classifying whether the material 102 is broken or whole. By way of another example, the valve 408 may be turned to induce tumbling of the material 102. This may allow imaging of the material 102 from different angles as the material 102 rotates, which may offer a more comprehensive shape and length measurement. Multi-frame views enable improved classification accuracy, especially for borderline or irregular kernels, by allowing fusion of multiple observations to assess continuity and overall kernel integrity.

In embodiments, the optics device 406 includes lighting 706. The lighting 706 may be any type of lighting, but specifically strobable (e.g., having the capacity to produce a strobe effect) LED lighting and may illuminate within the chamber 702. Additionally, the lighting may be positioned within the chamber 702. Embodiments for the lighting 706 may include LED ring lights that surround the camera 708 and/or surround the top material valve inlet.

In embodiments, the optics device 406 includes one or more cameras 708. The cameras 708 may be coupled to the chamber 702 (e.g., the wall of the chamber 702). Additionally, the cameras 708 may be configured to image the material 102 inside the chamber 702. The cameras 708 may image the material 102 at more than one angle.

Material 102 may fall from a previous step or directly through the optics device 406 via the valve 704. The cameras 708 and lighting 706 may be synchronized with the activation of the valve 704, ensuring that high-speed, high-quality images are captured as the material 102 falls. The lighting 706 and cameras 708 may be precisely timed to the opening of the valve 704 to optimize image clarity and detail. The number of images taken may depend on both the sample size and gravitational flow rate, ensuring a complete digital representation of the material 102. Additionally, images may be captured from multiple angles to fully digitize the material 102, which may be especially important for identifying characteristics that appear only on specific sides or surfaces during the grading process.

The cameras 708 in the optics device 406 may be surrounded by light emitting diode (LED) ring lighting to provide consistent and high-intensity illumination within the chamber 702. Additional LED ring lighting may be positioned at the top and bottom of the optics device 406 to ensure that the falling material 102 is brightly and evenly lit from multiple directions, supporting the capture of high-quality images. Other types of lighting 706, such as LED strip lighting, halogen, or incandescent lighting, may also be considered. However, LED lighting may be optimal due to its low operating temperature intensity, and lower power requirements. Maintaining low temperatures may help prevent the drying out of the material 102, which could otherwise compromise measurement accuracy.

Synchronized strobe lighting may be used in coordination with one or more cameras 708 to enhance image quality. These strobes may be timed to activate precisely with the camera shutters, providing the necessary lighting conditions for machine learning-based image analysis. Additionally, the strobes and cameras 708 may be synchronized with the valve 704 at the top of the instrument so that high-quality images of the falling material 102 can be captured at the exact moment it passes through the opening, improving consistency and accuracy in visual assessments.

It is further noted that the lighting 706 may include backlighting and front-lighting. The backlighting and front-lighting illumination techniques may enable continuous, high-resolution image acquisition of the material 102 as it moves through the chamber 702. Backlighting may be employed to enhance contrast and silhouette definition of the material 102, while front-lighting may provide complementary reflectance information. The integration of both illumination modes may enable robust detection and characterization of material 102 attributes in real time within a continuous flow environment. Backlighting illumination may provide critical visual contrast necessary for the automated detection and analysis of specific material 102 characteristics (e.g., fractures and breakage), where backlit images may produce clear edge contours and internal shadowing to facilitate identification of kernel fractures, cracks, and incomplete grains.

Germ identification may be supported by capturing differential translucency between germ regions and the endosperm through backlit imaging, allowing precise detection and localization within individual pieces. Additionally, by measuring transmitted light intensity profiles, the optics device 406 may quantify whiteness or translucency attributes to enable classification of the material 102 by opacity categories. Contrast between organic matter and non-grain inclusions may be enhanced through silhouette visualization, potentially improving contaminant detection accuracy. This continuous imaging approach may ensure that each piece of material 102 is individually analyzed without requiring batch separation, which could increase throughput and minimize manual handling.

Backlighting may be used to automatically generate segmentation masks for machine learning dataset creation. Specifically, backlit images may be subjected to edge-detection algorithms such as adaptive thresholding or Canny edge detection, exploiting the pronounced silhouette contrast to produce pixel-accurate masks corresponding to individual grain outlines. These auto-generated masks may be stored alongside raw image data to form structured datasets suitable for supervised learning workflows, with metadata labels (e.g., fracture status, germ presence, or whiteness level), provided either by an operator or parallel automated classification modules. The masks and labeled images may be partitioned into training, validation, and test datasets to enable reproducible model development for deep learning architectures like convolutional neural networks, which may be used to further refine grain grading algorithms.

In some embodiments, the backlighting module may be synchronized with a conveyance mechanism regulating grain flow through the imaging chamber, ensuring consistent orientation, and spacing of kernels while reducing motion blur. The system may optionally trigger backlight pulses coordinated with high-speed camera exposures to optimize image clarity and reduce power consumption.

This system may offer several operational advantages, including high-contrast, noise-resistant imaging, where backlighting could significantly improve segmentation accuracy compared to front-light-only systems, especially for translucent or irregularly shaped grains. Automated dataset creation may reduce labor requirements and increase the volume and consistency of training data by eliminating manual masking. Furthermore, the illumination, imaging, and data labeling components may be designed with scalable and modular architecture, potentially adaptable to other particulate materials beyond grain, such as seeds or legumes.

To address challenges associated with airborne dust generated by falling material, the cameras 708 may be designed for quick and easy lens cleaning. Additionally, camera lenses may be treated with a nanotechnology coating to reduce dust accumulation and maintain image clarity over time.

Additionally, air may be used to form an air curtain to protect the cameras 708 from dust. When applied to a camera lens, an air curtain may act as a protective shield that helps keep debris away without the need for physical covers or frequent cleaning. A small fan or compressed air system may direct a thin, continuous flow of air across the surface of the lens, creating a barrier that pushes away dust, water droplets, and other small particles before they can settle. This airflow may deflect airborne contaminants, such as fine dust or moisture, which is particularly advantageous in outdoor or industrial environments where such interference is common. By keeping the lens cleaner for longer periods, the air curtain may reduce the need for maintenance and physical wiping, which can lead to scratches or wear over time. Additionally, because it does not involve a physical barrier like a glass cover, the air curtain may preserve optical clarity and help maintain sharp, undistorted images. It is also contemplated that a camera 708 may be used within the optics module to view the status of the imaging lens, for the purpose of determining if lens cleaning is necessary. It could be possible for a pressurized air line to pulse and blow clean air at the lens to blow dust off and/or clean the lens.

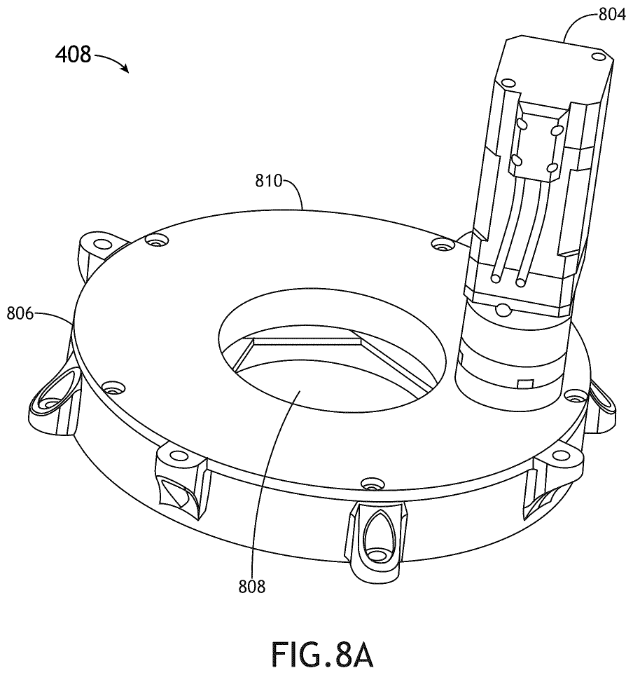

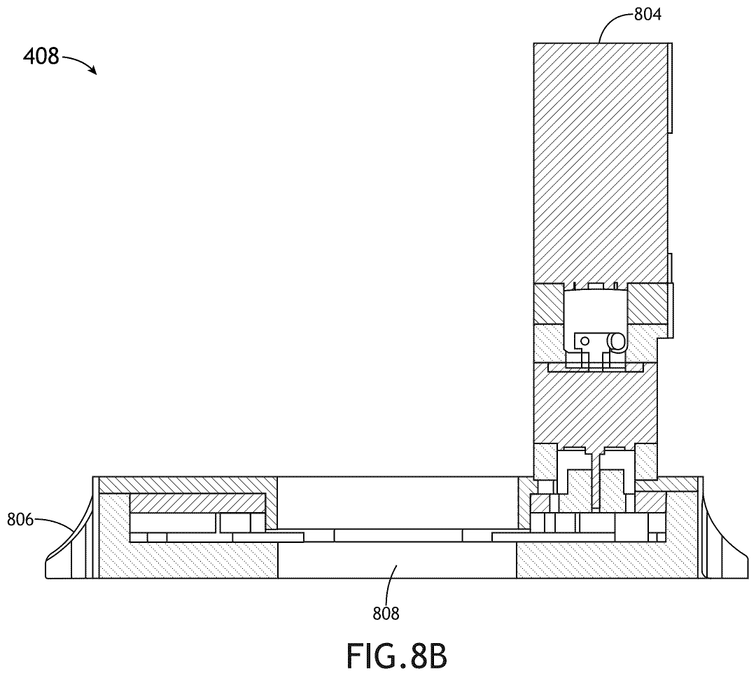

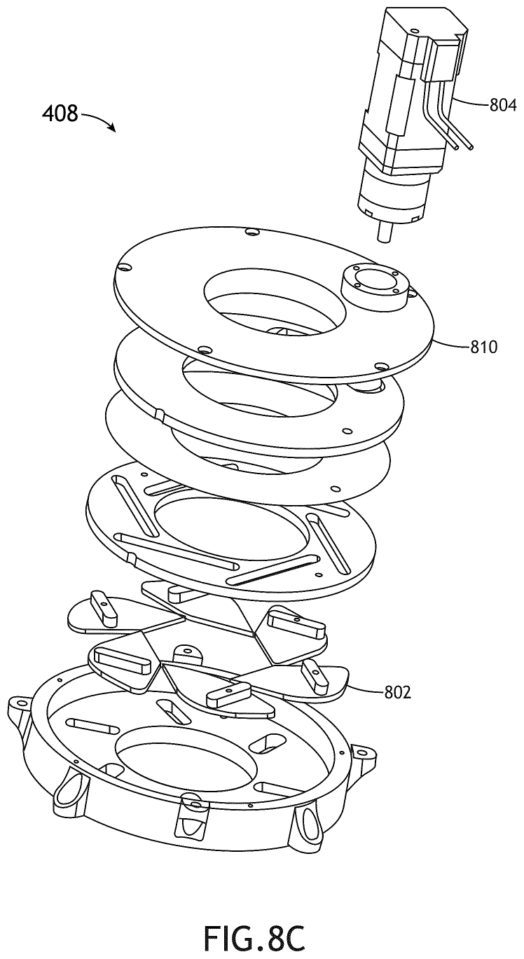

In embodiments, the continuous grading system 400 includes one or more valves 408. A valve 408 may be any mechanical device that controls the flow (e.g., restricts the movement) of materials, light, or fluids using a set of overlapping, retractable blades arranged in a circular pattern. It is noted that any valve discussed in the present disclosure may be a valve 408 as shown in FIGS. 8A-8C. Referring now to FIGS. 8A-8C, the valves 408 are discussed in greater detail. FIGS. 8A-8C illustrate the valves 408, in accordance with one or more embodiments of the present disclosure. It is noted that the valve 408 shown in FIGS. 8A-8C may be an iris gate, though that should be interpreted as illustrative rather than limiting. Alternatively, the valves could be rotating valves at one or more locations in the automated grading system, where one or motors rotate a closing mechanism, perpendicular to the direction of the material flow, for the purpose of providing an even flow to the next step of the process. A less useful, but also possible valve solution might utilize a traditional translational valve, using a slide gate.

In embodiments, the valves 408 include a plurality of blades 802. The blades 802 may be positioned in a circle and at least partially overlap. The blades may be curbed and thin pieces.

In embodiments, the valves 408 include an actuator 804. The actuator 804 may be any manual, electric, pneumatic, or hydraulic system that moves the blades 802.

In embodiments, the valves 408 include a housing 806 with a central hole 808 and a ring structure 810. The housing 806 may be a circular frame that holds the blades 802 and guides their motion. The blades 802 may also rest on the housing 806. When actuated, the blades 802 may slide inwards or outwards simultaneously, increasing or decreasing the size of a central hole 808. When fully closed, the blades 802 form a tight seal, while the fully open position provides an unobstructed passage through the central hole 808. Benefits of the valve 408 include self-cleaning capability. Another benefit includes that valves 408 are small, resulting in space saving.

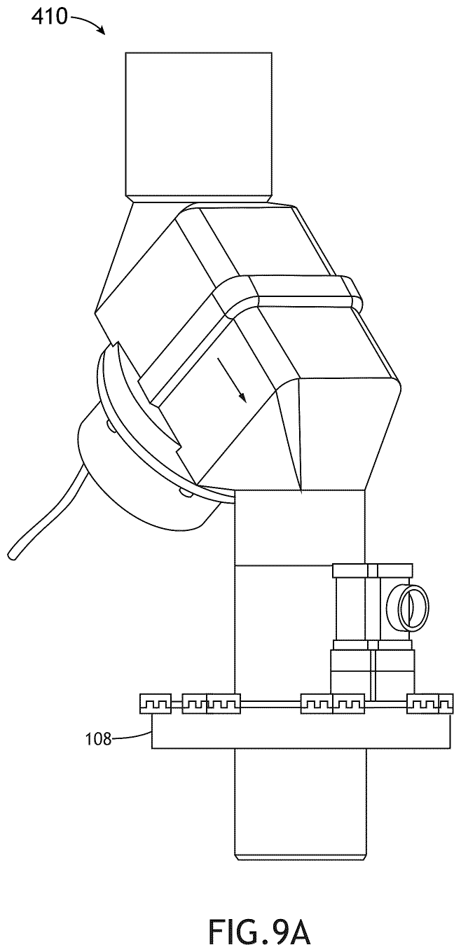

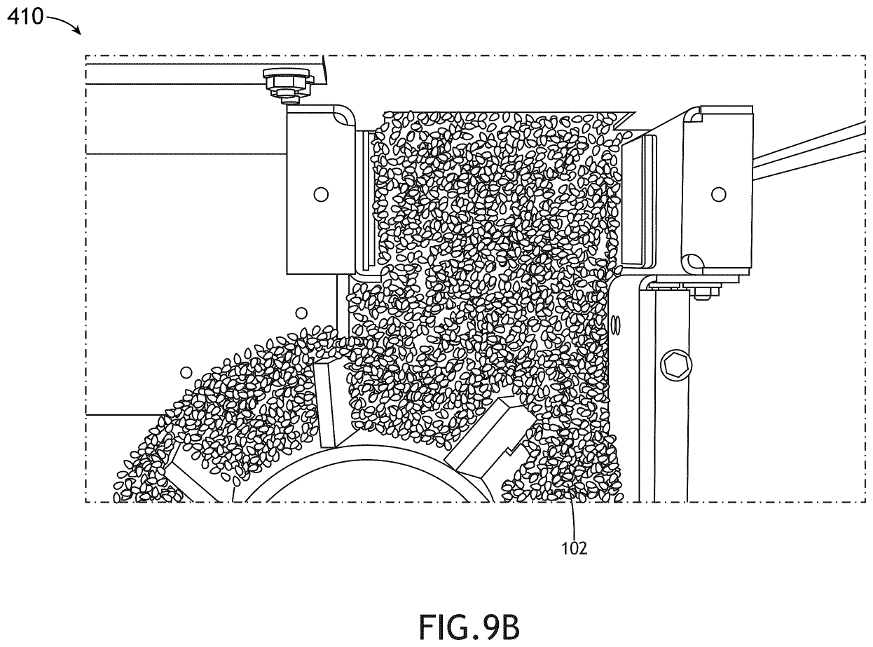

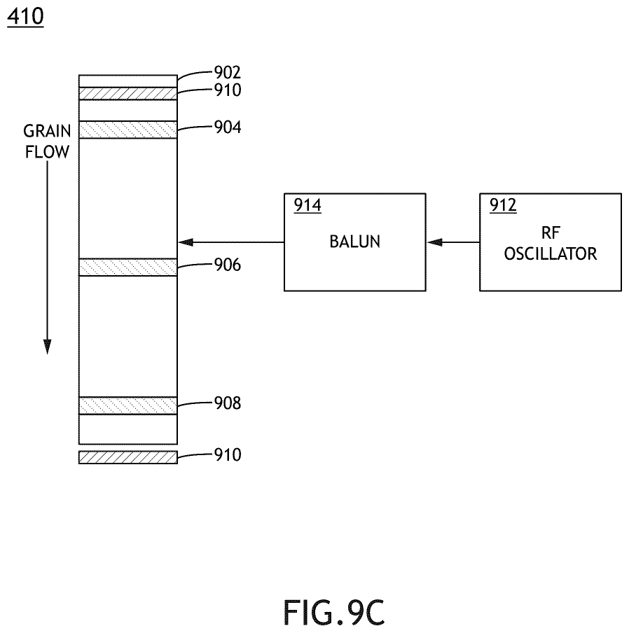

In embodiments, the continuous grading system 400 includes a moisture sensing device 410. The moisture sensing device 410 may determine a moisture content of the material 102 based on a cross section of the material 102. Referring now to FIGS. 9A-9C, the moisture sensing device 410 is discussed in greater detail. FIGS. 9A-9C illustrate the moisture sensing device 410, in accordance with one or more embodiments of the present disclosure.

The moisture sensing device 410 may be positioned within the continuous grading system 400 in a way that enables a full cross section of the material 102 to be available to the sensing instrument and enough sample movement to occur to enable an accurate measurement. For example, a valve 408 may close and the material 102 fills up inside of the moisture sensing device 410. The in-line moisture sensors typically will begin measurements once the chamber has become full of material. The microwave-type sensor can penetrate the moving grain to take readings. These readings can be averaged to obtain a single moisture reading or used individually.

Capacitance moisture sensing may require a grain sampling tube 902, accurately directing grain flow from top to bottom. The wider tube representation emphasizes physical symmetry, and grain flows past three conductive rings positioned along the tube: the top ring (RF+) 904, center ring (GND) 906, and bottom ring (RF−) 908. Static drain rings 910 are located at both ends to manage charge buildup.

The moisture sensing device 410 may include a radio frequency (RF) oscillator 912 that produces a fixed-frequency signal, which is fed into a balun 914 to create a differential RF output. This output drives a sensor comprising a pass-through, obstruction-free tube encircled by three axially aligned conductive rings, two outer rings (e.g., the top ring (RF+) 904 and bottom ring (RF−) 908) driven differentially and a center ring (GND) 906 held at virtual ground, forming a symmetric electric field. This may be part of a varactor-tuned LC resonant circuit that presents a frequency-dependent impedance. A directional coupler may monitor both forward and reflected RF power, with its outputs digitized by an analog-to-digital converter (ADC). Meanwhile, a digital-to-analog converter (DAC), accessed via a shared 12C bus with the ADC, adjusts the varactor bias, allowing the system to sweep through resonance frequencies.

Control logic may identify the resonance condition by detecting the minimum reflection coefficient, and correlate shifts in this condition to changes in material moisture, using a baseline air-filled reference for comparison. The moisture sensing device 410 may include additional features such as a programmable LED to indicate calibration or measurement status, a self-calibration routine that compensates for oscillator drift, and the use of coplanar waveguide with ground (CPWG) traces to route RF signals while contributing to the tank circuit's inductance. The DAC tuning voltage, buffered by op-amps and also sampled by the ADC, may provide a secondary reference for resonance detection. The directional coupler is implemented using a monolithic integrated circuit with logarithmic outputs, and all electronics are powered by a regulated direct current (DC) supply derived from a buck converter followed by a low-dropout regulator for noise reduction. This design supports configurability in tube diameter and length without altering the RF signal topology, ensuring adaptability across various applications.

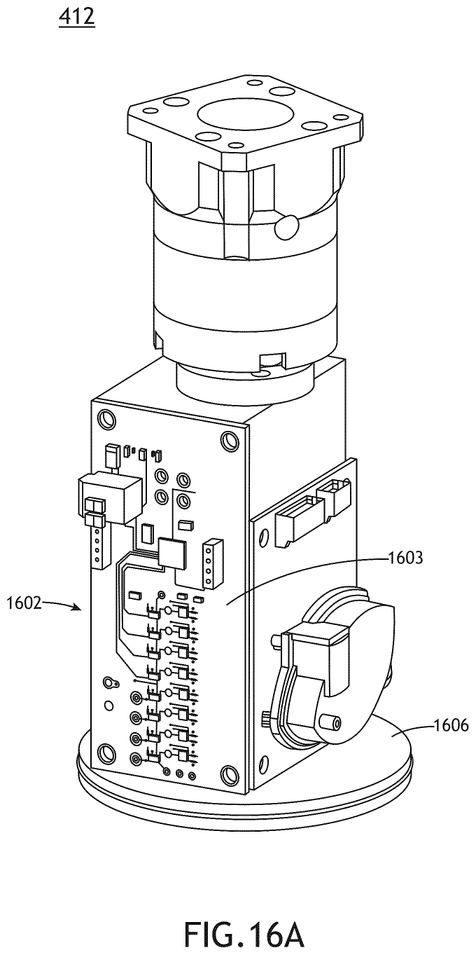

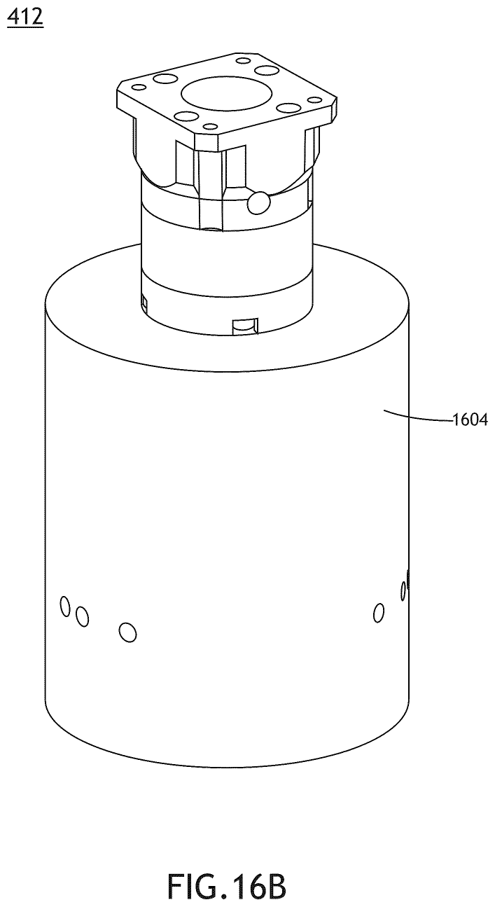

In embodiments, the continuous grading system 400 may include an odor sensing device 412. The odor sensing device 412 may determine one or more odor profiles of the material 102. The odor profile may determine a type of the material 102, a spoilage of the material 102, or one or more chemicals mixed with the material 102, or the like. Referring now to FIGS. 16A and 16B, the odor sensing device 412 is discussed in greater detail. FIGS. 16A and 16B illustrate the odor sensing device 412, in accordance with one or more embodiments of the present disclosure.

In embodiments, the odor sensing device 412 includes an odor sensor array 1602. The odor sensor array 1602 may include multiple, identical sensor modules 1603.

In embodiments, the odor sensing device 412 includes a cover 1604. The cover 1604 may be configured to at least partially cover the odor sensor array 1602.

In embodiments, the odor sensing device 412 includes a carousel 1606. The odor sensor array 1602 may be mounted on the carousel 1606, with each sensor module 1603 of the odor sensor array 1602 mounted behind its own protective cover and airflow port. At any given time, sensor module 1603 of the odor sensor array 1602 may be indexed into the active sampling position (e.g., air drawn past its sensing elements), while the others remain protected from contaminants. The sensor modules 1603 of the odor sensor array 1602 may be periodically rotated (e.g., on an annual or other predetermined interval) to bring a fresh sensor module 1603 into service. This approach may increase the overall service life of the array (e.g., four one-year sensor modules 1603 yielding a four-year lifespan), allowing cross-validation of readings by sampling the same airstream with two or more modules before and after rotation. Additionally, this approach may support automatic isolation (“masking”) of any sensor module 1603 whose output drifts beyond acceptable bounds. By combining replaceable cartridges with in-line validation, the odor sensing device 412 delivers both longer maintenance intervals and higher confidence in detection accuracy.

The entire odor sensing device 412 may be pre-packaged within a weather-tight, transportable cover 1604 (e.g., a container or a compact cabinet), which may simplify installation requirements. On-site setup may require only a level pad (e.g., a concrete pad) for odor sensing device 412 placement, conduit stub-outs through the pad or surrounding walls for power and vacuum (or compressed air) connections, and a utility stub for network or data connectivity. Power, vacuum intake and exhaust, and Ethernet (or other digital I/O) connections may terminate at standardized bulkhead fittings, either on the exterior of the cover 1604 or within a scale-house-mounted cabinet version. These fittings are typically quick-disconnect types, which may allow the cover 1604 to be easily swapped or serviced without modifying the facility's fixed utilities.

The system may be delivered as a complete, ready-to-operate grain-probe solution or as a modular addition to an existing probe installation. In locations where outdoor space is limited, a smaller version may be installed inside an existing scale house. By enclosing the pump, vacuum plumbing, sensors, control systems, and networking hardware within a single self-contained unit, the system may significantly reduce commissioning time, potentially to just a few hours, while eliminating the need for custom field wiring or civil work beyond concrete pad preparation and stub-out installation.

Odor sensing devices 402 may be positioned in one or more locations where odor detection is considered critical. Furthermore, the odor sensing devices 412 can be used together with other sensing modules or stand-alone by itself. These odor sensing devices 412 may use technologies such as electrochemical sensors, metal oxide semiconductor sensors, or photoionization detectors to detect and quantify VOCs associated with odors. As air flows across the odor sensing device 412, they may detect changes in VOC concentration. The measurement process is may be triggered by the engagement of one or more fans that pull data across the VOC sensors. The continuous grading system 400 may utilize either active sampling, where a fan draws a consistent airflow through the odor sensing device 412, or passive sampling, relying on the natural airflow within the automated material 102 grading system.

Once an odor is detected, the odor sensing device 412 may convert chemical signals into electrical signals. The odor sensing device 412 may use algorithms to analyze the data and distinguish between various odors based on their unique chemical signatures and concentrations. The processed data may be calibrated against predefined thresholds to determine whether the detected odor levels fall within acceptable limits or require further action. Alternatively, the raw data may be transferred to a central controller or sent to the cloud for analysis.

An odor sensing device 412 may feature a circuit board with embedded odor sensors exposed to an incoming airflow. Air may be drawn from a stream, such as a stream of material 102, using a fan assembly positioned outside the sensing unit, which pulls air through a dedicated fitting. A protective cover may help direct the material around the sensor array to avoid direct contact while still enabling odor detection. Such an assembly may also have broader applications beyond material 102 grading, including detecting illness in animal production environments, monitoring dust levels in areas at risk of explosion, or identifying the presence of drugs or hazardous materials at inspection points. Furthermore, it is contemplated that clean air may be blown across the sensing assembly to remove VOCs from the chamber and to minimize the chance for contamination from a previous sample.

The odor sensing device 412 may be designed to isolate the odor of the material 102 by preventing other odors from entering. After the material passes, the odor sensing device 412 may be exposed to a known odor in order to prevent trailing effects of the material 102 and check the calibration of the sensor against a known odor.

The odor sensing device 412 may be configured to determine if the odor profile of the material 102 indicates the material is an incorrect material. If incorrect, the continuous grading system 400 may be configured to prevent mixing with the correct material 102.

The odor sensing device 412 may be trained to identify a specific odor signature or odor profile, including target odors (e.g., odors that may pertain to characteristics of the material 102) in the presence of background odors.

In embodiments, the continuous grading system 400 includes a near-infrared (NIR) sensor 414. The NIR sensor 414 may perform spectroscopy on the material 102.

NIR sensing may offer a range of benefits when applied to the analysis of the material 102. The sensor 414 may allow for rapid, on-site testing, potentially improving throughput in processing facilities and enabling faster decision-making. Because NIR spectroscopy is non-destructive, the original material 102 may be preserved for further testing if needed. The technique may simultaneously assess multiple nutritional components, such as moisture, protein, fiber, fat, oil content, and carbohydrates, mycotoxin levels, ash content, or the like, thereby providing a comprehensive profile from a single analysis.

By reducing the dependency on extensive lab testing and delivering quick results, NIR sensing may help lower operational costs, especially in large-scale operations handling high volumes of material 102. The consistency and accuracy of NIR may support reliable quality assurance and regulatory compliance. Additionally, this sensing method may be integrated into the continuous grading system 400, to enable continuous, real-time monitoring that minimizes human error and streamlines workflow. Real-time data could allow for immediate process adjustments to optimize feed quality and animal performance. Since NIR sensing is chemical-free and non-destructive, it may also reduce environmental impact. Furthermore, data derived from NIR sensing may support predictive maintenance and improved inventory management by forecasting material 102 quality over time.

In embodiments, the continuous grading system 400 includes a mycotoxin sensor 416, either using the NIR sensor, ultraviolet lighting and camera, or using a lab in a box type module. The mycotoxin sensor 416 may determine a presence of mycotoxin in the material 102.

A series of one or more optical cameras may be synchronized with the activation of an valves 408. When the valves 408 opens, ultraviolet lighting and cameras may work in tandem to capture high-speed, high-resolution images of the falling material 102. The valves 408 opening may be adjusted to a specific size depending on the type of material 102 being evaluated, allowing a controlled volume of material 102 to pass through. The timing and number of images captured may be calibrated based on gravitational flow to ensure that a sufficient number of images are taken to digitally represent part or all of the sample, depending on system settings. Images may also be captured from multiple angles to create a comprehensive digital model of the sample, which may be important for assessing characteristics present on the backside of the kernels, such as indications of mycotoxin contamination. A grinding module may be utilized when ground material is needed to obtain the mycotoxin levels on the internal parts of the kernels that cannot be imaged from the exterior. Grinding may not always be necessary, and only necessary if toxins are not visible from the exterior.

If potential mycotoxin traits are identified during this image analysis step, the continuous grading system 400 may redirect the sample to undergo grinding. Because mycotoxins are often embedded within the material 102 and not always visible externally, grinding the material 102 to a specified particle size may expose internal contamination. The ground material 102 may then be analyzed using a mycotoxin sensor 416 to more accurately assess the internal presence of mycotoxins, supporting a more complete and reliable grading process.

In embodiments, the continuous grading system 400 includes a bagging device 418. The bagging device 418 may be configured to bag, or pack, graded material and label the graded material based on the grade.

In certain scenarios, a bagging device 418 may be implemented to streamline the process of bagging samples for analysis or verification of graded a material 102 that has been graded. The bagging device 418 may be integrated into the continuous grading system 400 and may involve multiple coordinated components to automate the bagging and labeling sequence. Initially, segregated material 102 samples may be transported to a designated bagging module through various methods, such as air conveyance, conveyor belts, augers, or other transport mechanisms. As the material 102 reaches the bagging module, sensors may detect the appropriate sample quantity, helping ensure each bag is filled consistently and accurately. The material 102 may then be dispensed into preformed bags. The bags may be composed of materials like polyethylene or kraft paper, chosen to preserve sample integrity.

The bagging device 418 may have precise measurement controls to ensure that each bag receives the designated amount of material. After filling, the bags may proceed to a sealing module, where they are securely closed using methods such as heat sealing, stitching, or adhesives, depending on the bag material. Sealed bags may then move to a labeling station, where automated label applicators generate and affix labels that contain key details such as material type, sample weight, batch number, packaging date, and potentially relevant quality certifications. Labels may include barcodes or QR codes to facilitate efficient tracking and inventory management.

The bagging device 418 may be designed to maintain the integrity of the material 102 for further testing, verification, or archival purposes. It may also include sensors and feedback mechanisms that trigger alerts in the event of discrepancies or equipment malfunctions. Overall, the bagging device 418 may enhance operational productivity, reduce labor input, and support traceability while maintaining the reliability of the grading and analysis of the material 102.

In embodiments, the continuous grading system 400 further includes an aflatoxin detection device 420.

The moisture sensing device 410 may begin by transporting material 102 samples from the storage or processing area. Automated sampling devices might extract small, representative portions at regular intervals without disrupting the overall material flow. Once collected, these samples may be transferred to an automated pre-processing unit, where they could undergo grinding and homogenization. In this step, solvents such as methanol or water with extraction agents may be automatically introduced within a fully enclosed environment to extract aflatoxins from the material 102 without requiring manual intervention.

Following extraction, the sample may be passed into an automated detection module that utilizes advanced analytical technologies. These may include fluorescent immunoassay systems that employ antibodies binding specifically to aflatoxins and emit measurable fluorescent signals, or Near-Infrared (NIR) spectroscopy, where spectral analysis combined with machine learning algorithms might estimate aflatoxin concentrations through reflected light signatures. Electrochemical sensors may also be used, detecting aflatoxin levels based on their interaction with sensor substrates and providing automated readouts in real time. Alternatively, the automated mycotoxin system could employ the use of disposable or permanent test strips that utilize chemistry to determine mycotoxin levels.

The resulting data may be processed by integrated software that determines aflatoxin levels, compares them against safety thresholds, and categorizes the material accordingly. Based on this analysis, the aflatoxin detection device 420 may automatically sort the material 102 into appropriate categories, such as suitable for use or requiring additional processing, and could trigger alerts or automated removal of contaminated samples. Detailed records may be generated and stored for quality control, traceability, and regulatory compliance. Additionally, the aflatoxin detection device 420 may function as part of the continuous grading system 400, offering real-time aflatoxin risk management to maintain the safety and quality of material 102 throughout the handling and processing stages.

In embodiments, the continuous grading system 400 includes a controller 114 communicatively coupled to any components therein. In embodiments, the controller 114 includes one or more processors 116. For example, the one or more processors 116 may be configured to execute a set of program instructions maintained in a memory 118. For example, the program instructions may be configured to cause the processors 116 to execute one or more steps of the present disclosure. In embodiments, continuous grading system 400 includes a user interface 120 communicatively coupled to the controller 114.

For example, the program instructions may be configured to cause the processors to receive data regarding the weight of the material 102, images of the material 102, moisture content of the material 102, and the odor profile of the material 102. Based on the received the weight of the material 102, images of the material 102, moisture content of the material 102, and the odor profile of the material 102, the processors may grade the material 102. It should be noted that the data may also include, mycotoxin presence, protein content, oil content, ash content, damage, foreign material, or the like.

Additionally, the processors 116 may be configured to prevent unwanted mixing of the material 102 based on the material type or the material grade (e.g., with coordinated series of gates, conveyors, motors, grain handling equipment, or the like). Additionally, based on the continuous grades produced by the continuous grading system 400, information may be communicated to harvesters, which may determine areas of the field to harvest, harvesting speed, or the like. Traceability of the material 102 (e.g., via identifiers 122) may be utilized in order to communicate with the correct harvesters.

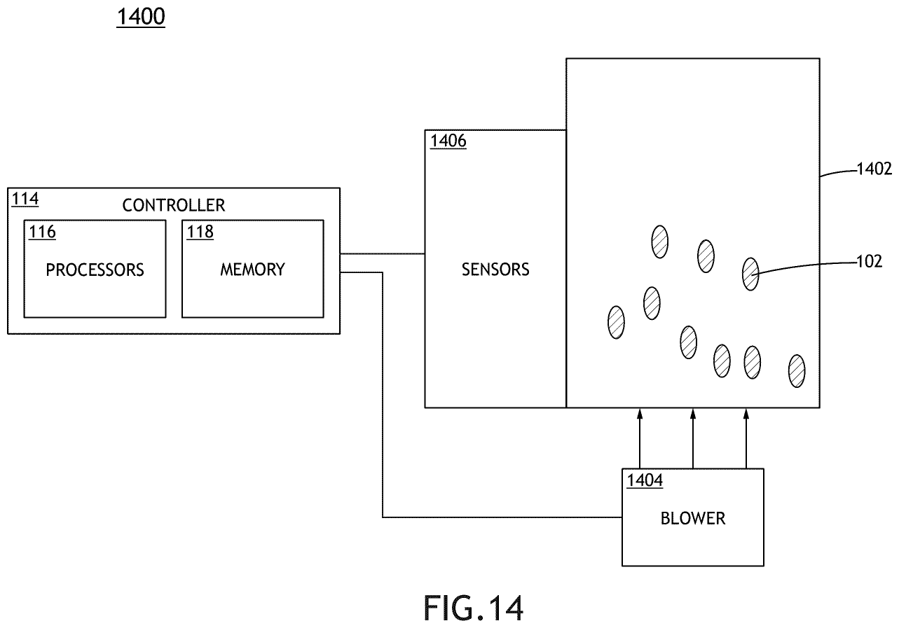

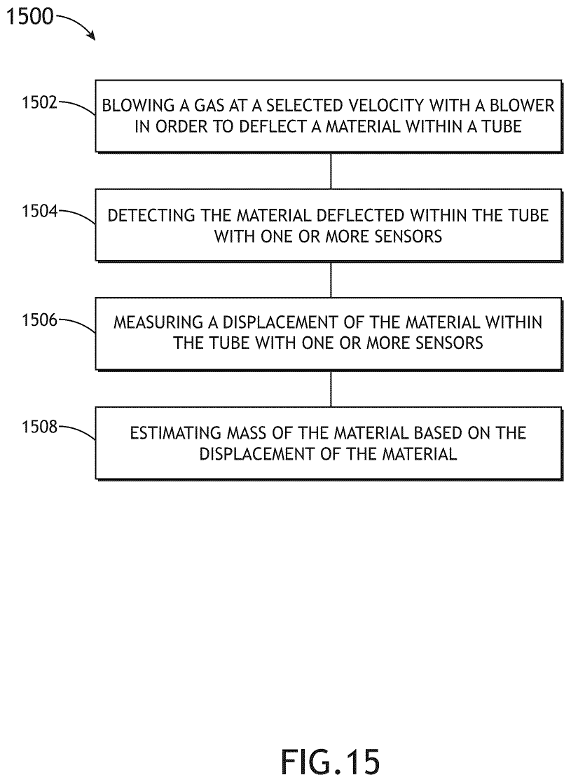

In embodiments, the continuous grading system 400 includes a mass estimating device 422. Referring now to FIGS. 14 and 15, the mass estimating device 422 is discussed in greater detail. FIG. 14 illustrates the mass estimating device 422, in accordance with one or more embodiments of the present disclosure. FIG. 15 illustrates a flow diagram of a mass estimating method 1500, in accordance with one or more embodiments

In embodiments, the mass estimating device 422 includes a tube 1402. The tube may contain a material 102. The material 102 may be mixed with one or more foreign materials within the tube.

The mass estimating device 422 may need to detect and account for foreign material to minimize their impact on mass estimation and quality assessments. These extraneous materials may include cob parts (e.g., irregularly shaped fragments that may disrupt airflow dynamics and affect weight measurements; leaves and leaf fragments, which may exhibit large surface areas and distinctive aerodynamic behavior that could be misclassified without proper trajectory analysis) and dirt clods, which may appear under wet conditions and possess similar mass profiles to larger grains, requiring optical methods for identification. Non-target seeds from weeds or other crops may also be present, potentially overlapping in physical characteristics with the intended grain but differing in grading significance. Insect parts, such as wings or larvae, may introduce erratic deflection patterns that interfere with measurement accuracy and may be relevant for food safety evaluations. Additionally, other agricultural debris like string, chaff, husks, or plastic shreds may enter the mass estimating device 422 and may distort flow patterns or sensor readings if not appropriately excluded.

It is noted that, depending on the grading standard, foreign material is either included with the material 102 during grading or must be removed from the material 102 before grading. For those grains requiring foreign material-free grading, a separation device (e.g., separation device 402) may be used to strip out foreign material prior to the mass estimate. All separated foreign material may then be measured and logged alongside the graded sample so we capture both “clean” material weight and the foreign material fraction.

In embodiments, the mass estimating device 422 includes a blower 1404. The blower 1404 may be any device known in the art configured to blow a gas (e.g., a fan or one or more jets). The gas may be blown at a selected velocity, a selected frequency, or for a selected duration. The gas may be blown in a laminar manner.

In embodiments, the mass estimating device 422 includes one or more sensors 1406. The sensors may include an optical sensor (e.g., cameras or high-speed imaging systems) or laser displacement sensors (e.g., to capture the deflection arc, angle, or displacement trajectory of material 102 after exposure to the air jet).

It is noted that the mass estimating device 422 may be configured to measure the deflection of the material 102 and/or individual particles within the material 102.

In embodiments, the mass estimating device 422 includes a controller 114 communicatively coupled to any components therein. In embodiments, the controller 114 includes one or more processors 116. For example, the one or more processors 116 may be configured to execute a set of program instructions maintained in a memory 118. For example, the program instructions may be configured to cause the processors 116 to execute one or more steps of the present disclosure. In embodiments, the mass estimating device 422 includes a user interface 120 communicatively coupled to the controller 114.

The controller may be configured to receive the displacement of the material 102 and/or individual particles within the material 102. from the one or more sensors and estimate mass of the material based on the displacement of the material. A physics-informed model or neural network may interpret deflection data relative to airflow parameters to estimate mass of the material 102 and/or individual particles within the material 102. The model may account for air resistance, drag coefficient variation by material geometry, and/or compensate for flow speed and material orientation. Calibration may be performed dynamically using known references or occasional sampling for ground truth validation.

FIG. 15 illustrates a mass estimating method 1500, in accordance with one or more embodiments of the present disclosure.

In embodiments, the mass estimating method 1500 includes a step 1502 of blowing a gas at a selected velocity with a blower in order to deflect a material within a tube. For example, the blower may include fans or jets. The gas may also be blown for a selected duration or a selected frequency. The aspects of the gas may be chosen based on the material being measured. The material as a whole, or individual particles within the material, may be deflected.

In embodiments, the mass estimating method 1500 includes a step 1504 of detecting the material deflected within the tube with one or more sensors. For example, the one or more sensors may include optical sensors (e.g., cameras or high-speed cameras). The one or more sensors may also be a laser displacement sensor (e.g., to measure a position of the material within the tube). The deflection may also be detected for individual particles within the material.

In embodiments, the mass estimating method 1500 includes a step 1506 of measuring a displacement of the material within the tube with one or more sensors. For example, the blower may blow the material a certain distance. That distance may be measured in order to determine one or more characteristics of each particle within the material. The foreign material may also be deflected a different distance from the material based on the displacement, which would translate into a different particle density and mass. The displacement may also be measured for individual particles within the material.

In embodiments, the mass estimating method 1500 includes a step 1508 of estimating mass of the material based on the displacement of the material. The estimation may be based on the displacement of the material (e.g., because heavier material will be displaced less than light material). The estimation may correct for air resistance, drag coefficient variation by material geometry, and/or compensate for flow speed and material orientation. The mass may also be determined for individual particles within the material.

In embodiments, the continuous grading system 400 includes one or more redundant modules 424. The redundant modules 424 may be arranged in series or parallel with any other component of the continuous grading system 400. Additionally, the redundant modules 424 may provide redundancies for any component of the continuous grading system 400. In this way, the redundant modules 424 may include a redundant separation device 402, a redundant weighing device 404, a redundant optics device 406, a redundant moisture sensing device 410, a redundant odor sensing device 412, a redundant NIR sensor 414, a redundant mycotoxin sensor 416, a redundant bagging device 418, a redundant aflatoxin detection device 420, and/or a redundant mass estimating device 422. The redundant modules 424 may be utilized to gain capacity, provide redundancy for potential failures, or check results.

It is noted that the various components of the continuous grading system 400 may be modular. In this way, multiple modules that can physically be added together. For example, any of the components described herein may be stacked upon each other. Such a configuration may provide for movement of the material 102 by gravity.

Module control and networking may be configured in multiple ways depending on operational needs, including individual control, networked configurations, or hybrid arrangements combining both. Each module may operate independently, providing precise, localized control suitable for isolated tasks, manual testing or calibration, and standalone automation based on predefined inputs or logic. Alternatively, modules may be networked through wired or wireless communication protocols to enable centralized or distributed control. This setup may allow for synchronized operations across multiple modules, scalability through easy addition of new components, and remote monitoring and control via a centralized system or cloud interface. In some implementations, modules may be paired or grouped in hybrid configurations to meet specific performance goals. These pairings may include arrangements where several modules function as a single unit to increase capacity, hierarchical control structures with primary and secondary roles, or dynamic reconfiguration that allows modules to alternate between standalone and networked operation. This modular approach may offer flexibility, scalability, and adaptability in complex systems involving material 102 processing, grading, or handling.

Further, the continuous grading system 400 may offer significant customizability. Grading requirements for the material 102 may vary significantly based on a range of factors, and the use of annotations may enable fine-tuning of grading models to suit specific needs. Each facility might use different grading modules or quality standards, requiring site-specific annotations of images or odor profiles to account for local variations. Such annotations may help mitigate inconsistencies caused by environmental conditions unique to that location. Similarly, regional grading algorithm customization may be necessary due to differences in regulations, climate, material size, material shape, or market expectations. In these cases, annotations gathered from different regional sites may allow the algorithm to adjust for localized defects, preferred sizes, or maturity levels, supporting consistent grading within a region while acknowledging natural variability.

Crop-level customization may also be beneficial, as different crops exhibit distinct characteristics such as texture, shape, or defect thresholds. Personnel might annotate specific defect types, size parameters, or ripeness indicators for each crop, enabling the grading system to apply tailored models that improve accuracy and reduce classification errors. On a company level, organizations may have proprietary standards for size, color, texture, or defect tolerance. Feedback from customers could be incorporated via annotation, allowing the grading system to align with evolving business requirements and client expectations. Specifically, various customers might define different docking mechanisms based upon the automated grading data. It is likely that FGIS (Federal Grain Inspection Services), the grading arm of the United States Department of Agriculture (USDA) will have specific requirements for export-bound grain grades. State weights and measures departments will likely also have specific standards that must be met.

As part of a continuous learning process, the grading system may evolve through ongoing annotation, correction, and reinforcement. Personnel might review and adjust model outputs, ensuring the system remains current with seasonal trends, shifting quality definitions, or emerging customer needs. The benefits of this annotation-based customization approach may include greater accuracy, improved operational efficiency through automation, enhanced customer satisfaction due to tailored quality assessments, and scalable deployment across various crops and sites through cloud-connected infrastructure.

FIG. 4D illustrates material 102 entering the continuous grading system 400, in accordance with one or more embodiments of the present disclosure.

The continuous grading system 400 may be installed onto a conveyor that transports material 102 from the container 104 dumping location to one or more storage areas at the site, which may include steel bins, concrete silos, flat storage buildings, or temporary pile-based storage. The continuous grading system 400 may be integrated into various types of equipment, such as conveyors, augers, sorters, or distributors, to automatically direct the movement of material 102 throughout the facility. It may facilitate the transfer of material 102 from one or more storage containers to others, with the objective of sorting similar material into corresponding storage units. Additionally, the system may be used to blend material 102 from multiple storage sources to achieve targeted characteristics such as desired moisture content, foreign material thresholds, or odor profiles.

The continuous grading system 400 may automatically analyze one or more loads to determine the type of material 102, enabling automated sorting to the appropriate handling and storage locations. In this context, the continuous grading system 400 may serve as the central control unit that activates specific augers, conveyors, sorters, or other equipment to ensure that incorrect material is not mistakenly routed to the wrong storage area. Additionally, loads that fail to meet predefined specifications may be automatically directed to designated segregation storage structures based on the evaluation of the continuous grading system 400. The continuous grading system 400 may also incorporate Artificial Intelligence (Al) and Machine Learning (ML) to improve its performance over time by learning from large datasets. Furthermore, the continuous grading system 400 may integrate with RFID tags on container 104 units to automatically associate each load with its corresponding delivery vehicle and potentially even the field of origin, thereby supporting a comprehensive traceability framework.

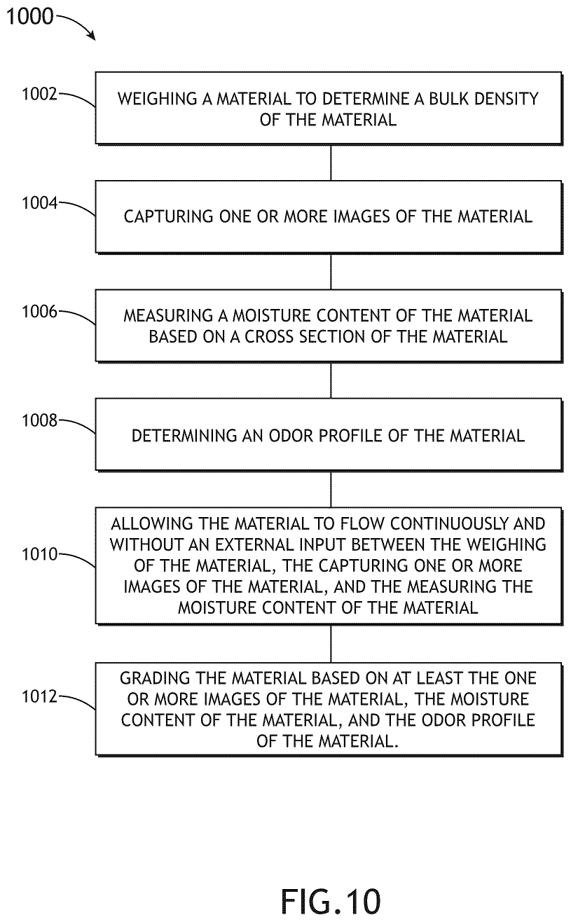

FIG. 10 illustrates a flow diagram of a continuous grading method 1000, in accordance with one or more embodiments of the present disclosure. Applicant notes that the embodiments and enabling technologies described previously herein in the context of the continuous grading system 400 should be interpreted to extend to continuous grading method 1000. It is further noted, however, that the continuous grading method 1000 is not limited to the architecture of the continuous grading system 400.

In embodiments, the continuous grading method 1000 includes a step 1002 of weighing a material to determine a bulk density of the material. For example, the material may be weighed in a known volume. In this way, a bulk density may be determined which will allow weight of the material to be determined from a volume of the material, and vice versa. Additionally, it should be noted that the material may be weighed along, or may be weighed along with any foreign material mixed with the material.

In embodiments, the continuous grading method 1000 includes a step 1004 of capturing one or more images of the material. It may be beneficial to capture images of the material after the foreign material has been separated in order to better analyze the material itself. However, it may also be beneficial to capture images of the material mixed with the foreign material in order to determine the amount and/or type of foreign material mixed with the material.

In embodiments, the continuous grading method 1000 includes a step 1006 of measuring a moisture content of the material based on a cross section of the material. For example, moisture content may be measured for the entire sample, including both the material and any foreign material.

In embodiments, the continuous grading method 1000 includes a step 1008 of determining an odor profile of the material. For example, it may be beneficial to determine an odor profile of the entire material, including any foreign material. It may be important to include foreign material when determining an odor profile because the foreign material may include one or more undesirable characteristics. The odor profile may include a type of the material, a spoilage of the material, or one or more chemicals mixed with the material.

In embodiments, the continuous grading method 1000 includes a step 1010 of allowing the material to flow continuously and without an external input between the weighing of the material, the capturing one or more images of the material, and the measuring the moisture content of the material. For example, the material may be able to continuously flow through the measurement steps without any external inputs, such as humans, robotic arms, or the like. The flow of the material may be controlled by valves, which may be automatically controlled. Further, because the measurements of the material may be performed with modular structures, the material may be measured in any order, may have the same measurements taken more than once, or may have additional measurements taken.

In embodiments, the continuous grading method 1000 includes a step 1012 of grading the material based on the one or more images of the material, the moisture content of the material, the damage of the material, and the odor profile of the material. All of these factors may be considered when generating a grade for the material. However, any other factors may be analyzed when generating the grade for the material in addition to the one or more images of the material, the moisture content of the material, the damage of the material, and the odor profile of the material.

In embodiments, the continuous grading method 1000 includes a step of performing spectroscopy on the material. Performing spectroscopy on the material may include determining at least one of moisture content, protein levels, fiber, fat, oil, ash, or carbohydrate content of the material based on the spectroscopy of the material. It is contemplated that a near-infrared sensor may be used to perform spectroscopy in order to change material storage characteristics and preserve the material.

In embodiments, the continuous grading method 1000 includes a step of determining a presence of mycotoxin in the material. For example, mycotoxins may be seen in images of the material. However, in order to determine mycotoxins, the material may need to be ground and further analyzed.

In embodiments, the continuous grading method 1000 includes a step of deflecting the material with a gas at a predetermined velocity to determine a mass of the material. For example, the foreign material may be lighter than the material. Therefore, the gas may be able to deflect the foreign material and measuring the respective deflections of various particles in order to determine the respective particles mass and be able to constantly determine mass of the foreign material. Additionally, the velocity of the gas may be automatically selected based on the target type of material.

In embodiments, the continuous grading method 1000 includes a step of separating the material from foreign material with a gas at a predetermined velocity. For example, the material may be lighter than the foreign material. In this way, a velocity of gas may be selected so it blows away any foreign material, while not discarding the material.

In embodiments, the continuous grading method 1000 includes a step of bagging graded material and labeling the graded material based on the grade. For example, an automated bagger may be used to bag the graded material. It should be noted that the material may be bagged so like grades are together.

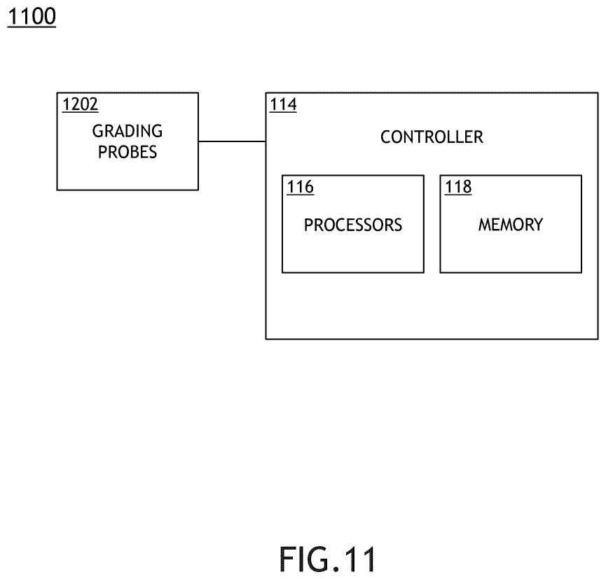

FIG. 11 illustrates a block diagram of a futures index generation system 1100, in accordance with one or more embodiments of the present disclosure.

In embodiments, the futures index generation system 1100 includes a network of grading devices 1102. network of grading devices 1102 may be any devices that are capable of grading a material 102. It is noted that the network of grading devices may include the grading system 100 or the continuous grading system 400 as described herein. However, the 1102 is not required to include the grading system 100 or the continuous grading system 400 and may include any type of grading devices 1102. Each grading device may be located at a different receiving point.

Each device may be deployed at a deployed at receiving points (e.g., grain elevators, cooperatives, processors, feed mill, rock quarry, fertilizer plant, seed facility, blending plant, flour mill, food processing plant, a grain terminal, a port, a ship, a railroad, or the like) captures standardized quality metrics for each material 102 delivery.

In embodiments, the futures index generation system 1100 includes a controller 114 communicatively coupled to any components therein. In embodiments, the controller 114 includes one or more processors 116. For example, the one or more processors 116 may be configured to execute a set of program instructions maintained in a memory 118. For example, the program instructions may be configured to cause the processors 116 to execute one or more steps of the present disclosure. In embodiments, the futures index generation system 1100 includes a user interface 120 communicatively coupled to the controller 114.

For example, the program instructions may be configured to cause the processors 116 to receive the grade of the material from each of the network of grading devices as grade data (e.g., moisture content, test weight, a presence of foreign material, damage to the material, or an odor). For example, the grade data may be transmitted wirelessly or through any other communication medium.

By way of another example, the program instructions may be configured to cause the processors 116 to aggregate the grade data. The grade data may be aggregated across multiple sites and timeframes, normalizing data formats and applying statistical weighting methods to generate composite quality scores for defined regions, crops, or other materials. Additionally, machine learning may be utilized in aggregating the grade data. It is noted that aggregation may occur in the cloud in order to enable real-time aggregation.

By way of another example, the program instructions may be configured to cause the processors 116 to generate an index, wherein the index is based on an average value of the material, wherein the average value of the material is based at least partially on the grade data. The index may represent the average quality-adjusted value of a given commodity over a defined time window and geography.

By way of another example, the program instructions may be configured to cause the processors 116 to construct a futures fund based on the index. The quality-based index may serve as the underlying benchmark for a tradable futures contract or exchange-traded fund (ETF), allowing market participants to speculate on, or hedge, against movements in aggregate material quality, rather than volume alone. Multiple indexes may be created for different materials or different defined geographies, specifically for materials such as commodity grains.

The futures index generation system 1100 may offer several advantages that enhance the overall efficiency and transparency of the agricultural market. By incorporating remotely-measured, real-time, objective quality data for the material 102 into pricing mechanisms, it may improve market accuracy and support better-informed decision-making. This advancement may also enable the development of new financial instruments, such as futures contracts and ETFs, which are based on quality-weighted indexes, potentially creating a new class of agricultural financial products.

Additionally, storage considerations may be taken into account for commodity pricing. For example, as more grain companies use remote grain condition monitoring technologies, it may be possible to gain a measurable appreciation of the stored grain across a wide distribution of the market. This data may be turned into knowledge about how much and how good of condition of grain is in storage across a region. This would be of value to tradeable markets.

Furthermore, producers and buyers may benefit from improved risk mitigation strategies, as these tools can facilitate hedging against regional or seasonal fluctuations in material quality. The integration of quality-based pricing may also incentivize the adoption of superior post-harvest practices, as higher-quality outputs could be rewarded with more favorable pricing. Lastly, the approach may be highly scalable and adaptable across different crop types and regions, enabling the creation of multiple indexes tailored to specific geographic zones and material characteristics.

Index weighting within this system may be configurable to reflect specific end-user needs, such as differentiating between the requirements of ethanol producers, milling operations, animal producers, and food processors.

To support real-time decision-making, a dynamic dashboard may be utilized to visualize live movements in quality indexes, offering stakeholders and investors accessible, up-to-date insights. Additionally, smart contracts or blockchain technology may be incorporated to ensure the transparency, traceability, and immutability of index data, thereby reinforcing trust in the system's outputs and supporting regulatory or commercial validation needs.

The widespread availability of regional and global grain and other materials grading data may fundamentally transform futures trading by introducing real-time, objective quality insights into pricing models that have historically relied on generalized supply and demand estimates. Traditionally, futures markets have struggled to incorporate variations in commodity usability or quality across regions. However, with the disclosed systems and methods, grading data can be aggregated and published in a standardized format, enabling new market mechanisms.

For example, futures pricing may begin to reflect regional quality differentiation, allowing contracts to account not only for volume but also for the relative value or usability of crops. A drop in quality in a specific region, such as increased damage, foreign material, mycotoxin levels, insect levels, lower test weights, or other characteristic such as mold, fungus, or virus, may influence local futures benchmarks. Additionally, localized hedging instruments may become viable, enabling cooperatives or processors to hedge against region-specific quality risk, such as that caused by drought or disease.

This granularity of data may also open up inter-regional arbitrage strategies. Traders may be able to capitalize on differences in regional quality indexes by taking long or short positions across zones showing divergent trends. Globally, such data may enable the creation of international quality benchmarks, supporting premium pricing for food-grade or export-quality products, and standardizing trade negotiations.

Further, predictive analytics using these datasets may provide early signals for supply chain disruptions, well ahead of traditional government reports. A trend like declining test weights in several regions could indicate future issues in ethanol, feed, four, or other supply chains, influencing futures behavior in advance.

Finally, regulators and policy-makers may benefit from this transparency, using standardized grading data to detect market irregularities, better manage food security, and implement targeted interventions during crises. Overall, the integration of high-resolution grain quality data into commodity markets may represent a significant advancement in market precision, efficiency, and fairness.

The integration of real-time mycotoxin detection into Automated Grain Grading systems may significantly alter the dynamics of national and international trade, especially for export-driven agricultural economies. By embedding sensors that detect contaminants such as aflatoxins, fumonisins, ochratoxins, and deoxynivalenol or vomitoxin (DON), exporting nations may gain the ability to generate high-frequency, verifiable data that supports more accurate quality indexing. This capability can position these countries more competitively in global markets.

Real-time sensing may enable nations to certify outgoing shipments as compliant with international food and feed safety thresholds, reducing the likelihood of border rejections and opening access to higher-value markets with stringent regulations, such as, but not limited to, the European Union, Japan, and South Korea. With a transparent and traceable quality assurance system, countries might command price premiums for clean exports.

Furthermore, exporters may dynamically route and segregate grain at the point of origin, separating contaminated or borderline grain from high-grade product. This capability may streamline operations, minimize cross-contamination risks, and ensure that grain is directed toward appropriate market destinations based on safety and quality thresholds.

From a policy and diplomacy perspective, countries with reliable mycotoxin surveillance infrastructures may leverage this transparency in trade negotiations. The ability to provide real-time, certified safety data could serve as a trust mechanism in bilateral and multilateral trade agreements, accelerating customs clearance processes and fostering deeper trade partnerships.

On a broader level, nations adopting such technologies may develop national branding initiatives, such as “Clean Grain Certified” or “Safety Verified Export,” that enhance their reputation in food safety and agricultural innovation. These brands may become globally recognized indicators of quality, boost export revenues, and strengthening soft power.

Domestically, the presence of real-time monitoring may contribute to public health by enabling early intervention during contamination outbreaks. It may also ensure the stability of local food and feed chains, while internationally, it could provide a consistent, reliable supply of safe commodities, support global food security, and reduce the volatility that contamination issues introduce into the grain trade.

Finally, the data derived from validated mycotoxin detection may be integrated into futures index construction. Grain with certified safety attributes might serve as a premium tier in pricing indexes or as a separate commodity class altogether. This may introduce a new hedging instrument in agricultural finance—enabling risk mitigation strategies specifically focused on contamination, in addition to general quality and quantity metrics.

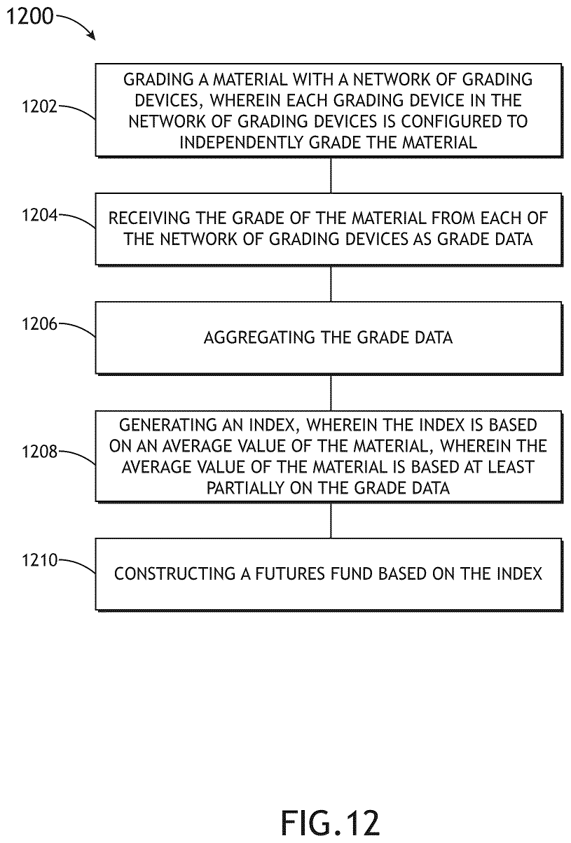

FIG. 12 illustrates a flow diagram of a futures index generation method 1200, in accordance with one or more embodiments of the present disclosure. Applicant notes that the embodiments and enabling technologies described previously herein in the context of the futures index generation system 1100 should be interpreted to extend to futures index generation method 1200. It is further noted, however, that the futures index generation method 1200 is not limited to the architecture of the futures index generation system 1100.

In embodiments, the futures index generation method 1200 includes a step 1202 of grading a material with a network of grading devices, wherein each grading device in the network of grading devices is configured to independently grade the material. It is noted that each grading device may be located at a receiving point. Additionally, each grading device may be located at a different receiving point (e.g., grain elevator, a cooperative, or a grain processor). Further, the grading devices may be automated grading devices.

In embodiments, the futures index generation method 1200 includes a step 1204 of receiving the grade of the material from each of the network of grading devices as grade data. For example, the grade data may be transmitted over a wireless connection or any other suitable data transfer connection(s).

In embodiments, the futures index generation method 1200 includes a step 1206 of aggregating the grade data. The grade data may be aggregated across multiple receiving points or multiple timeframes. Aggregating the grade data may also be done with machine learning.

In embodiments, the futures index generation method 1200 includes a step 1208 of generating an index, wherein the index is based on an average value of the material, wherein the average value of the material is based at least partially on the grade data. The index may be generated for a defined time and/or a defined geography. The index may also be adjusted for a known end-use of the material. Additionally, multiple indexes may be created for different materials or different defined geographies.

In embodiments, the futures index generation method 1200 includes a step 1210 of constructing a futures fund based on the index. Unlike traditional futures funds that are based on quantity, the futures fund generated by the present method may be based on quality and quantity. Such a futures index may result in heightened awareness of quality throughout regions.

In embodiments, the futures index generation method 1200 includes a step of generating quality scores for regions where the network of grading devices is located. For example, the quality scores may reflect how the quality of the material changes throughout different regions.

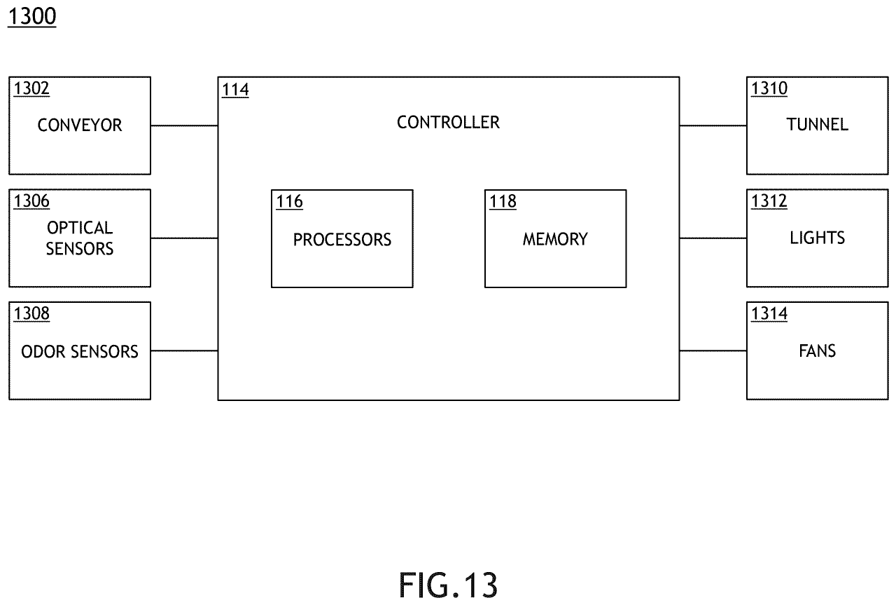

FIG. 13 illustrates a block diagram of a sorting system 1300, in accordance with one or more embodiments of the present disclosure.

In modern manufacturing and assembly lines, automated sorting systems may be used to help maintain efficiency and improve accuracy. These systems may leverage technologies such as computer vision, odor sensing, and various other measured metrics to identify, classify, and sort products or components. By automating the sorting process, manufacturers, including those in agricultural production, may reduce labor costs, minimize human error, increase throughput, and achieve more consistent product output.

One such technology that may be implemented is computer vision, which may utilize high-resolution cameras and image processing algorithms to detect subtle differences in shape, color, size, or texture. For example, in processing environments for vegetables, fruits, meat, or other food products, computer vision systems may detect defects or irregularities and remove non-conforming items from the line in near real-time, potentially resulting in more uniform shipments.

In addition to visual inspection, odor sensing may also be employed to detect volatile organic compounds, particularly in commodities like melons, pineapples, strawberries, or peppers, where freshness and aroma are critical quality indicators. Electronic noses equipped with arrays of odor sensors may analyze odor profiles and compare them to reference standards to assess product consistency.

Automated sorting systems may also incorporate other measurable properties, such as weight, density, and acoustic signatures. For example, weight-based sorting may ensure packaging consistency, and acoustic monitoring may help detect defects through variations in sound.

When multiple sensing modalities, such as vision, weight, and odor, are combined, sorting systems may gain improved precision and adaptability. These systems may also use machine learning algorithms to enable continuous learning and refinement, allowing them to adapt to new product variations and quality specifications over time.

In embodiments, the sorting system 1300 includes a conveyor 1302. The conveyor 1302 may be designed to move a material 102.

In embodiments, the sorting system 1300 includes an automated sorting device 1304. The automated sorting device 1304 may include robotic arms, pneumatic actuators, or conveyor diverters. The automated sorting device 1304 may be dynamically controlled by an automated system that interprets inputs from one or more sensors. As data from is processed in real time, the sorting system 1300 may automatically position the automated sorting device 1304 to guide each item to the appropriate bin or conveyor path. This may ensure accurate and efficient separation of material 102 based on criteria (or criterion) such as quality, type, or defect status.

In embodiments, the sorting system 1300 includes one or more optical sensors 1306. The optical sensors 1306 may be configured to capture one or more images of the material as it passes the optical sensors 1306 on the conveyor 1302.

The 1306 may be configured to perform hyperspectral imagery. Hyperspectral imagery (HSI) may be used as an advanced imaging technology that captures and processes information across a wide range of the electromagnetic spectrum. Unlike conventional imaging systems that typically capture three spectral bands (e.g., red, green, and blue) hyperspectral sensors may collect data across hundreds of narrow, contiguous spectral bands. This enhanced spectral resolution may allow for the detection of subtle differences in chemical composition, moisture content, and structural characteristics of materials, including agricultural commodities such as material 102. Potential benefits of using HSI for commodity grading may include more objective and consistent quality assessments by reducing reliance on manual grading and visual inspection; the capability for real-time or near-real-time screening of large volumes at facilities such as grain elevators or processing plants; early identification of contaminants or damage such as Fusarium or aflatoxin presence prior to visual manifestation; varietal classification of material 102, potentially distinguishing between hybrid and non-hybrid or genetically modified and non-genetically modified varieties based on their unique spectral signatures; and quantitative estimation of compositional attributes such as moisture and protein when combined with chemometric modeling. Recent improvements in sensor miniaturization, computational capabilities, and machine learning may make it feasible to deploy HSI in operational environments.

In embodiments, the sorting system 1300 includes one or more odor sensors 1308. The odor sensors 1308 may be configured to capture and odor profile of the material as it passes the odor sensors 1308 on the conveyor 1302.

In embodiments, the sorting system 1300 includes a tunnel 1310. To maintain consistency in image capture, the optical sensors 1306 may be housed within the tunnel 1310. This design may help eliminate variations caused by ambient lighting, dust, or other environmental factors. The tunnel 1310 may be constructed from lightweight, durable materials such as aluminum or stainless steel, often with matte or non-reflective coatings to reduce glare and unwanted reflections.

In embodiments, the sorting system 1300 includes one or more lights 1312. Inside the tunnel 1310, the one or more lights 1312 may be strategically placed to provide uniform illumination. High-intensity lights, such as LEDs, may be used due to their consistent brightness and low heat output, which reduces the risk of overheating the sensors or the material 102 being inspected. Depending on the application, lighting may be customized to include multispectral or hyperspectral sources to enhance the detection of subtle surface variations, contaminants, or defects that are not visible under standard lighting. The optical sensors 1306 may be mounted at specific angles within the tunnel 1310 to capture multiple perspectives of the material 102. High-speed cameras may be paired with line-scan or area-scan sensors, depending on the type of material 102 and the desired resolution. The optical sensors 1306 may be synchronized with a conveyor 1302 to ensure that the material 102 is scanned precisely as it moves through the tunnel 1310.

In embodiments, the sorting system 1300 includes one or more fans 1314. For odor sensing, maintaining a controlled and consistent airflow may be important. An effective way to capture odors from the produce may be by installing fans 1314 that direct air off the material 102 on the conveyor and into the 1308. These fans 1314 may create a gentle, directed breeze that moves air from the conveyor 1302 area into the odor sensors 1308.

To maintain consistent air quality within the tunnel 1310, the one or more fans 1314 may include filtered intake vents and exhaust fans to remove background odors or contaminants. This prevents cross-contamination and ensures that only the odors from the material 102 are analyzed.

The placement of fans 1314 and air ducts may be designed to avoid disrupting the movement of the material 102 while efficiently capturing the desired odor profile. In some setups, a vacuum or suction mechanism may be used instead of fans to gently pull air through a narrow passageway into the odor sensing chamber.

In embodiments, the sorting system 1300 includes a controller 114 communicatively coupled to any components therein. In embodiments, the controller 114 includes one or more processors 116. For example, the one or more processors 116 may be configured to execute a set of program instructions maintained in a memory 118. For example, the program instructions may be configured to cause the processors 116 to execute one or more steps of the present disclosure. In embodiments, the sorting system 1300 includes a user interface 120 communicatively coupled to the controller 114.

The systems and methods disclosed herein may have applications in numerous fields.

For example, the systems and method disclosed herein may have applications relating to grain quality. Utilizing odor sensing technology to distinguish a particular product may involve a systematic approach to capturing and analyzing its odor profile. The process may begin by collecting a wide array of product samples to establish a robust odor database, accounting for natural variations. These samples may then be analyzed using odor sensors, which detect and quantify volatile organic compounds (VOCs) that contribute to the product's unique odor signature. With this chemical profile established, machine learning algorithms may be employed to train models that recognize and classify the product based on its odor characteristics. This approach may support quality assurance, help authenticate branded goods, and assist in detecting counterfeits across various industries.

When applied to stored grain, such as agricultural commodities, odor sensing technology may offer several advanced quality control and operational benefits. For example, it may be used to automatically determine the age of stored grain by detecting odor profile shifts that correlate with degradation over time. This could unlock new marketing opportunities by assigning economic value to older, but still high-quality, material. Forward-looking grain marketing strategies may also benefit from odor sensing, enabling sellers to time their sales based on storage conditions, spoilage risk, or other odor-based quality parameters.

Additionally, the system may continuously monitor stored grain for odor-related changes, automatically alerting operators when the detected odor profile exceeds predefined thresholds indicative of spoilage or contamination. This data can feed into smart control systems that manage aeration fans, conveyance equipment (e.g., augers or conveyors), allowing real-time decisions to be made about aeration schemas, or where to send grain. For instance, material showing early signs of degradation may be automatically routed to a segregated storage bin to avoid contamination of high-quality inventory, or scheduled for more immediate sale. Alternatively, poor, and high-quality material may be blended to achieve desired average quality parameters.

By way of another example, the systems and method disclosed herein may have applications relating to insect infestation. Grain weevils, also referred to as granary weevils or rice weevils, may pose a significant threat to the quality and safety of stored grain. These small reddish-brown insects are known to lay their eggs directly inside individual kernels of grain, such as wheat, rice, oats, barley, or corn. As the larvae hatch and develop within the kernel, they consume the internal contents, causing structural damage and diminishing both nutritional value and marketability. This internal feeding process may result in substantial economic loss for storage operators and producers.

In addition to the physical destruction of the kernels, grain weevils may contaminate stored grain with fecal matter, shed skins, and other biological residues, leading to spoilage and making the grain unfit for human or animal consumption. Their activity may also promote the growth of mold and other harmful microorganisms, compounding the degradation and increasing the risk of mycotoxin development. This contamination often contributes to undesirable odors and flavors, which may render entire batches unpalatable and commercially unacceptable.

Weevil populations may proliferate rapidly within a storage environment, and once an infestation becomes established, eradication may prove difficult. As such, preventative measures are critical. Strategies may include maintaining rigorous sanitation protocols, implementing fumigation or insecticidal treatments where allowed, using sealed storage containers, and continuously monitoring environmental and biological conditions. Automated sensing technologies, including odor detection or visual inspection systems, may assist in the early identification of infestation indicators, enabling timely interventions. Addressing grain weevil risk is therefore essential to preserving the quality, safety, and economic value of stored grain throughout the supply chain.

By way of another example, the systems and method disclosed herein may have applications relating to rice. Each kernel's contour may be analyzed to extract measurements such as length, width, aspect ratio, and features like edge smoothness and truncation. Kernels that are shorter than approximately 75% of the calibrated whole-kernel mean length may be flagged as broken. Data processing may involve aggregating results over rolling windows, ranging from about 500 to 2,000 kernels, with the broken grain percentage calculated in real time. These broken grain statistics may be stored for traceability and displayed through local or remote user interfaces. In manual operation, operators may view real-time metrics and trends to potentially take corrective actions such as reducing roller pressure, adjusting gap settings, or modifying rice drying or tempering upstream. In automated systems, the grading module's output may be linked to the mill's control system to dynamically slow throughput to reduce impact forces, rebalance whitening versus polishing stages, or alert and adjust for mechanical issues like misaligned rollers or worn surfaces. This configuration flexibility may allow the grading system to be tuned for different grain flow characteristics, throughput requirements, and imaging goals.

The Kett whiteness estimation may be used to analyze the white ness of the rice. Kett whiteness values may be estimated using calibrated optical reflectance measurements, where kernels are imaged under full-spectrum white light or narrow-band multispectral sources. Reflectance intensity, particularly in visible and near-infrared bands, may be captured and processed using a whiteness estimation model. Model calibration may involve training a machine learning or regression model with ground-truth Kett meter readings across various rice samples with different polishing levels. The model may convert reflectance patterns into an estimated Kett whiteness index with precision typically within ±1-2 Kett units of the physical instrument. These estimated whiteness values may be streamed in real time and visualized on various interfaces. The data may be used to detect under-milling or over-milling, adjust abrasive or frictional milling stages dynamically, and track whiteness uniformity across batches.

The benefits of real-time broken and whiteness detection may include increased yield by minimizing broken grain and preserving more marketable rice, improved energy efficiency by avoiding over-polishing and reducing machine wear, enhanced quality control through immediate feedback allowing tighter control over product consistency and compliance, and automation readiness by integrating these parameters into closed-loop feedback systems for full or semi-automated milling optimization.

By way of another example, the systems and method disclosed herein may have applications relating to livestock. Livestock heat detection and optimized breeding timing may be supported through a deeper understanding of the estrus process, during which female livestock animals become sexually receptive and fertile. This reproductive phase may involve various hormonal and behavioral changes, primarily influenced by rising estrogen levels leading to ovulation. A key characteristic of estrus may be the emission of specific smells or pheromones that indicate the female's reproductive status to male animals. These chemical cues, often present in bodily secretions, may attract potential mates, and assist in synchronizing breeding behaviors.

During estrus, female animals may produce increased vaginal mucous secretions that contain pheromones, potentially giving off a distinct odor detectable by male animals even from a distance. Alongside olfactory changes, females may exhibit behavioral shifts such as increased vocalizations, restlessness, and mounting behavior; all of which may serve to signal their fertility and increase mating likelihood. Urinary odors may also play a role, as males are often highly sensitive to pheromonal markers in female urine that indicate ovulation timing and fertility readiness.

Pheromones may also be emitted through skin, breath, or other bodily secretions and may function to attract males and promote coordinated reproductive activity within a herd. Additionally, some females may engage in scent-marking behaviors, such as urinating or rubbing against surfaces to deposit pheromones in shared spaces.

By recognizing these olfactory and behavioral indicators, livestock producers may improve breeding efficiency, reduce the need for invasive hormonal treatments, and enhance reproductive outcomes. Technologies that detect these pheromonal emissions, such as odor sensors or electronic noses, may provide real-time monitoring of estrus status, enabling more precise breeding decisions and supporting optimized reproduction strategies in commercial livestock operations.

The birthing process in animals may represent one of the most vulnerable stages

in a production cycle, requiring timely oversight to protect the health and survival of both mother and newborn. Complications such as dystocia, improper fetal positioning, or maternal exhaustion may arise suddenly, and without prompt intervention, these events may result in the loss of the newborn, the mother, or both. Traditional monitoring methods, which often rely on human observation during periodic checks, may be insufficient in large-scale operations where the ability to provide individual attention to each animal is limited. To address this, a range of technological systems may be employed to deliver continuous, near real-time monitoring and early warning of birthing-related complications.

Vision sensing may be used to detect behavioral indicators of labor onset or complications. High-definition cameras may be installed in birthing areas to provide constant visual surveillance, and night vision or thermal imaging capabilities may be added to ensure monitoring even in low-light or nighttime conditions. These imaging systems may assist in detecting subtle changes in posture, activity level, or interactions between animals that signal the need for intervention.

Wearable sensors may track physiological data such as heart rate, temperature, and movement, offering insight into the mother's condition. Deviations from expected patterns may trigger automated alerts to caretakers. In parallel, sound-based monitoring systems may use microphones to capture vocalizations, which may be analyzed by pattern recognition software to distinguish between normal and distress-related sounds, further enabling timely responses. Additionally, calming music or natural soundscapes may be played in the birthing area to reduce environmental stress and support smoother labor.

Odor sensing systems may complement these methods by detecting specific pheromonal or biochemical changes in the mother's bodily emissions that signal imminent labor. These electronic noses may recognize scent patterns imperceptible to humans, enabling earlier detection of parturition onset. Furthermore, automated scent dispensers may deliver calming essential oils to the environment, promoting comfort and reducing anxiety in laboring animals.

When livestock animals are ill, various odors may be present that can serve as indicators of underlying health issues. These smells may arise due to metabolic changes, infections, digestive disturbances, or other physiological imbalances. Recognizing and interpreting such odors may support early detection of disease, allowing for timely veterinary intervention and improved herd health management. For instance, foul odors may result from bacterial infections, abscesses, or tissue necrosis, often indicating the presence of decaying or infected tissue. A persistent ammonia-like smell may be associated with high concentrations of urea or ammonia due to poor waste management, inadequate ventilation, or urinary tract issues. In cases of metabolic disorders such as ketosis animals may emit a sweet or fruity odor in their breath or urine due to elevated ketone levels. Digestive disorders like bloat may cause malodorous emissions due to fermentation gases, while diarrhea, depending on its cause, may be accompanied by foul, acidic, or putrid smells. Wounds or necrotic tissue may release odors reminiscent of decomposition, signaling infection or tissue death. In other cases, a strong urea smell may indicate kidney dysfunction, urinary infections, or imbalances in nitrogen metabolism. Odor-based monitoring, particularly when integrated with sensor technologies, may provide a valuable, non-invasive tool for detecting illness and maintaining animal welfare in livestock operations.

By way of another example, the systems and method disclosed herein may have applications relating to animal feed. The sense of smell plays a critical role in the feeding behavior and overall well-being of livestock animals. Their highly developed olfactory systems may influence appetite, food selection, and nutritional intake, making scent a key driver of feed interaction. The aroma of feed may attract animals, encouraging them to approach and consume their rations with more enthusiasm, especially when the odors are pleasant and appetizing. Familiar scents may help animals recognize specific feed types or ingredients, guiding their food preferences based on previous experiences and nutritional needs. Additionally, livestock may use smell to assess feed quality, detecting spoilage, contamination, or freshness issues that may not be visually apparent. Off odors might cause animals to avoid spoiled or unsafe feed, helping reduce health risks and digestive issues. Appealing aromas can also enhance the sensory experience of feeding, supporting better intake and growth performance by improving feed palatability and reducing stress-related feeding problems. Given the importance of scent in livestock feeding, odor sensing technologies may be employed to verify feed identity and quality. For example, feed recipes or rations may be assigned unique odor signatures, enabling automated systems to detect mismatches or confirm that the correct feed is being delivered to the correct bin, building, or site. Odor sensors may be positioned on feed truck bins, the truck stinger, or on the feed bins themselves to monitor and validate feed distribution. Such implementations may help prevent cross-contamination, reduce ration delivery errors, and support more precise livestock nutrition strategies. By incorporating odor sensing into feed management, producers may improve feed efficiency, animal health, and overall farm productivity. Odor sensing can be combined with other fool-proofing technologies such as GPS, RFID, QR codes, bar codes, etc. and in combination with feed bin level sensing solutions in order to ensure that the right feed is delivered to the right location.

Feed recipes or rations may be assigned unique odor signatures, allowing automated systems to detect and alert responsible managers when discrepancies or exceptions occur. These odor-based confirmations may also be tied to individual feed truck loads, ensuring that the correct formulation is being delivered. To prevent the wrong feed from being installed in the wrong bin, odor confirmation may be implemented at multiple points in the distribution chain. For instance, odor sensors may be installed in each bin of a feed truck, on the stinger used to deliver the feed, or on the receiving feed bins themselves.

By way of another example, the systems and method disclosed herein may have applications relating to dockage management. Maintaining the right ratio of good quality grain to poor quality grain is essential for livestock farmers to stay within target dock percentages and optimize feed efficiency. By carefully balancing the inclusion of high-quality grains with lower-quality grains in feed formulations, farmers can manage dockage deductions effectively and minimize economic losses associated with feed quality discrepancies.

Dockage refers to the deductions or discounts applied to the price of grain to account for foreign material, high moisture content, damaged kernels, or other quality factors. By blending good quality grains with poorer quality grains in the right proportions, farmers can mitigate the impact of dockage on overall feed costs and maintain profitability.

Balancing good grain with poor quality grain allows farmers to optimize feed costs while maintaining feed quality and nutritional value. By strategically managing the ratio of grains in feed formulations, farmers can minimize the impact of dockage deductions on overall feed expenses and maintain cost-effective feeding practices.

The right blend of good quality and poor-quality grains can help maintain a balanced nutritional profile in livestock feed. While high-quality grains provide essential nutrients and energy, lower-quality grains may still contribute to overall feed volume and bulk. Careful formulation of feed rations ensures that animals receive the necessary nutrients while managing feed costs and quality considerations.

Creating the right ratio of grains in feed formulations also helps farmers diversify their feed sources and mitigate risks associated with fluctuations in grain quality and availability. By incorporating a mix of grain types, farmers can reduce reliance on a single grain source, optimize resource utilization, and adapt to changing market conditions while staying within target feed ration percentages.

By emphasizing the importance of balancing good grain and poor-quality grain in livestock feed formulations, farmers can effectively manage dockage deductions, maintain feed quality standards, optimize feed costs, and support healthy growth and performance in their animals. Strategically blending grains to achieve the desired target dock percentages contributes to sustainable feeding practices, efficient resource utilization, and profitability in livestock production.

By way of another example, the systems and method disclosed herein may have applications relating to unsafe entries. Unsafe entry into industrial, livestock, or agricultural sites poses significant risks to workers, farm personnel, and the environment due to the presence of hazardous substances such as chemicals, anhydrous ammonia, methane in manure, and other potentially dangerous compounds. Exposure to these substances can lead to respiratory issues, skin irritation, chemical burns, poisoning, and, in severe cases, fatalities. Implementing a proactive system to mitigate such risks is essential for safeguarding the health and safety of individuals working in these environments. More importantly, keeping unauthorized personnel and vehicles out of livestock sites/buildings helps to keep unwanted diseases out of livestock areas.

Livestock facilities generate methane gas as a byproduct of manure decomposition. Methane is highly flammable and poses risks of explosion or asphyxiation if not managed properly. Installing effective ventilation systems, monitoring methane levels, and following safety guidelines for manure handling and storage are vital steps to minimize methane exposure hazards and enhance worker safety.

To mitigate risks associated with unsafe entry into sites containing hazardous substances, a proactive system may include thorough risk assessments, the implementation of safety protocols and standard operating procedures, comprehensive personnel training, clear warning signs and physical barriers, monitoring tools for detecting gas leaks or toxic fumes, and emergency response plans for accidents or spills.

One effective way to enhance safety is through the use of smart and connected sensors that detect gases such as CO2, methane, anhydrous ammonia, and other noxious substances like mold spores in enclosed grain storage or livestock structures. These sensors can operate on a frequent monitoring schedule, providing proactive alerts or notifications to workers to prevent entry into unsafe spaces. Additionally, data from pit levels may be incorporated with the presence of gases.

Specific applications of odor sensing technology include monitoring the odor signatures of anhydrous ammonia tanks and measuring ammonia (NH3) levels in the environment to issue timely warnings. Sensors can verify airspace safety in chemical storage facilities, fertilizer structures, manure-containing livestock buildings, and chemical loading/unloading areas to detect leaks or unsafe conditions.

Data collected from these sensor arrays can be transmitted to cloud platforms for automated alerting via mobile devices or processed on-site to trigger immediate notifications through lighting or other local alert systems.

By integrating such advanced odor and chemical sensing technologies into safety protocols, organizations can significantly reduce the risk of hazardous exposure, protect worker health, ensure regulatory compliance, and maintain safer working environments in industrial, livestock, and agricultural settings.

By way of another example, the systems and method disclosed herein may have applications relating to monitoring manure constituents. Manure constituent sensing may hold considerable value in modern agriculture because it could play an important role in nutrient management, environmental stewardship, and operational efficiency. Manure, a byproduct of livestock production, may contain essential nutrients such as nitrogen, phosphorus, and potassium that could be beneficial for crop fertilization. However, improper application or excessive accumulation of manure might lead to nutrient runoff, water contamination, and greenhouse gas emissions, which may pose environmental risks and regulatory challenges.

One key reason for the potential value of manure constituent sensing is precision nutrient management. Accurate measurement of nutrient concentrations in manure may allow farmers to better adjust application rates to meet crop requirements. This could help optimize nutrient uptake by crops, reduce waste, and improve fertilizer efficiency. Monitoring nitrogen, phosphorus, and potassium levels in manure may also support more balanced fertilization practices, which might promote crop growth while minimizing environmental impacts such as nutrient leaching and runoff.

Environmental protection is another important consideration. Manure runoff containing excess nutrients may contribute to eutrophication of water bodies, potentially causing algae blooms and degradation of aquatic ecosystems. Sensing technologies might help farmers manage nutrient applications to mitigate these risks. In addition, monitoring ammonia and other volatile compounds in manure may help reduce emissions that contribute to air pollution and odor problems, potentially improving air quality in rural communities.

Compliance with regulations may be facilitated through manure sensing as well. Many regions may have strict requirements regarding manure management and nutrient application to protect water quality and public health. Sensing technologies might provide accurate data on nutrient content and application rates, helping farmers meet these regulatory standards.

Operational efficiency and economic factors could also benefit from manure constituent sensing. Optimizing nutrient management through precise sensing may reduce fertilizer costs and minimize losses from inefficient use or overapplication of manure. Properly managed nutrient cycles may support healthy soil fertility and crop yields, which could contribute to sustainable agricultural practices and long-term economic viability.

Data-driven decision-making is another potential advantage. Continuous sensing and data collection may enable farmers to make informed decisions in real-time, adjusting management practices based on current nutrient levels and environmental conditions. Additionally, manure sensing technologies may integrate with precision agriculture systems, potentially enhancing overall farm management and decision-support tools for sustainable intensification.

Overall, remote sensing of manure constituents may provide both economic and environmental benefits. Economically, it may allow for more precise nutrient management, reducing the need for commercial fertilizers and lowering input costs. By applying manure more efficiently, farmers might maximize crop yields and improve soil health, leading to increased profitability. Environmentally, remote sensing technology could help prevent over-application of nutrients, minimizing runoff and protecting water bodies from eutrophication and pollution. It may also assist in monitoring and controlling emissions of volatile compounds such as ammonia, reducing air pollution and odors related to manure management. Furthermore, this technology might support compliance with environmental regulations, helping to avoid potential fines and improve sustainability credentials. Taken together, the integration of remote sensing for manure constituents may promote both economic efficiency and environmental stewardship, contributing to more sustainable agricultural practices.

By way of another example, the systems and method disclosed herein may have applications relating to agronomy. Knowing whether chemicals may have been applied to an agricultural field before humans enter it could be important for several reasons. This information may help protect workers from exposure to potentially harmful pesticides, herbicides, or fertilizers, which could cause acute or chronic health concerns. Access to chemical application data may also support compliance with safety regulations and re- entry intervals, reducing the likelihood of unintended exposure. In addition, understanding when chemicals may have been used might prevent accidental environmental contamination, particularly in nearby water sources or sensitive non-target areas. Furthermore, this awareness may allow for better coordination of field operations, enabling workers to plan around safe entry times and potentially improving overall productivity. Altogether, such knowledge may foster a safer working environment, help meet regulatory expectations, and support more efficient farm management.

Periodic odor measurements in agricultural fields may provide useful insights for proactive intervention and planning. Sensors that detect volatile organic compounds (VOCs) and other odors associated with crop health, pest presence, or disease development may enable real-time environmental monitoring. Shifts in odor profiles might indicate potential problems such as fungal growth, pest infestations, or nutrient deficiencies, possibly before visual symptoms are evident. Early identification of such issues may allow for timely responses, including irrigation adjustments, targeted pesticide or fertilizer application, or use of biological controls. When integrated with other environmental data, odor sensing may help farmers design informed operational strategies that promote crop health, enhance yields, and minimize losses, contributing to more sustainable agricultural practices.

The use of remote analytics applied to sensor data in agriculture may enhance decision-making, improve efficiency, and support sustainability. These systems may enable a more integrated and responsive approach to farm management. Combining inputs from multiple sensors (e.g., soil, weather, crop health) may offer a more complete picture of field conditions. Data fusion techniques might improve the reliability and accuracy of these insights.

Machine learning algorithms could analyze historical sensor data to forecast trends in pest activity, crop growth, or soil nutrient dynamics. Such models may help optimize timing for irrigation, fertilization, and other inputs. Statistical tools may identify irregularities in sensor data that could indicate emerging threats such as disease outbreaks or equipment malfunctions. Early detection may allow for preventive action.

Geospatial tools may be used to understand how soil characteristics, pest presence, or nutrient levels vary across fields. This analysis might support targeted input use and site-specific management.

Interactive dashboards may provide real-time displays of sensor data along with recommendations. DSS platforms may include tools to detect recent chemical applications and suggest safe re-entry times, monitor nitrogen volatilization through odor or chemical signals, or track regenerative practices to verify organic compliance.

Internet of Things (IoT) platforms may allow farmers to remotely monitor and control systems like irrigation, pest detection, and environmental sensors. This may help reduce labor and enhance operational efficiency.

Crop simulation models may combine environmental and sensor data to estimate yield potential under various conditions. This information may guide planting densities, harvest scheduling, and nutrient management. In addition, technologies such as crop odor sensors or vision systems may help determine crop growth stages remotely, enabling timely input decisions.

By way of another example, the systems and method disclosed herein may have applications relating to hazard monitoring. Hazard monitoring in the grain storage industry may play an important role in supporting the safety of stored grain and the continuity of facility operations. Grain storage environments can present a range of hazards that, if not appropriately monitored, may result in safety incidents, operational disruptions, or financial losses. By implementing systems that track environmental and mechanical conditions, facilities may be better equipped to mitigate risks and maintain grain quality over time.

Temperature fluctuations within grain bins may create favorable conditions for mold development, insect infestations, and spoilage. Hot spots, which may arise due to inconsistent airflow or uneven moisture distribution, could cause localized grain heating and increase the potential for combustion. Managing temperature across the entire bin may help reduce these risks.

Moisture content is another critical factor that may affect grain stability. Excess moisture may accelerate mold growth and fungal contamination, possibly degrading the grain's nutritional quality and market value. In some cases, elevated moisture levels could also cause grain to cake or clump, potentially compromising the structural stability of the storage system.

Gas buildup may also occur as a result of biological activity within stored grain. This process may lead to the accumulation of gases such as carbon dioxide and methane, while simultaneously reducing oxygen levels. Inadequate ventilation under such conditions may increase risks to personnel, including asphyxiation, and may heighten the chances of spontaneous combustion.

Grain dust, which often accumulates during handling and storage, may pose a serious explosion hazard. Under the right conditions, this fine particulate matter could ignite, leading to potential damage, injury, or loss of life. Dust management practices and proper hazard detection technologies may help mitigate this risk.

Operational systems such as motors and bearings may be vulnerable to overheating, especially with continuous operation or mechanical friction. Without proper monitoring, elevated temperatures in these components could result in equipment failure or fires. Bearings, in particular, may wear down due to environmental conditions, improper lubrication, or sustained load pressures. This wear and tear may not only interrupt operations but also pose safety risks if the equipment fails unexpectedly.

Electrical hazards may also exist in the form of faulty wiring, short circuits, or degraded insulation. These conditions may lead to fires or electrical shocks, potentially endangering both personnel and equipment within the facility.

Odor detection systems may help identify ozone or other chemical markers associated with overheated motors and bearings. Such early detection could allow operators to intervene before mechanical components fail.

Some systems may combine odor detection with vibration sensors and thermal imaging to enhance the accuracy of monitoring efforts. These integrated tools may provide a more complete view of equipment health and environmental risks.

Automated alerting systems may also be implemented to notify facility managers when early signs of potential failure are detected. These warnings may offer lead time for preventive maintenance or operational adjustments, reducing the chances of equipment breakdowns or safety events.

By adopting these sensing and alerting technologies, grain storage operations may improve their ability to monitor risk factors and respond proactively to emerging threats, ultimately supporting safer, more efficient storage environments.

By way of another example, the systems and method disclosed herein may have applications relating to harvesting. A smart harvesting solution may offer considerable advantages for agricultural operations by using near real-time data and interconnected technologies to guide harvesting decisions. When combine harvester settings are adjusted based on live data from grain dryers and storage facilities, the harvesting process may become more efficient and responsive to changing conditions. This adaptive approach may help reduce downtime, improve throughput, and potentially enhance grain quality by minimizing factors like foreign material and cracked kernels, which may otherwise result in docking fees at grain elevators. Additionally, when grain is harvested with more uniform moisture content, it may reduce the energy and cost required for drying, contributing to more sustainable resource use and improved farm profitability.

To support this process, a structured flow of operations may be implemented. It begins with data collection and communication across the system. Combine harvesters may already be equipped with sensors to monitor variables such as grain moisture, yield, and protein levels. Enhanced on-board grain grading systems may also provide additional insights by analyzing foreign material, cracked grain, color, and other quality indicators in real-time, allowing for more refined control of harvester settings. Such a grain grading system would likely be installed in or near the clean grain elevator on a combine. Meanwhile, sensors in grain dryers and storage facilities may measure incoming grain quality, including moisture and contamination. Smart trucks, fitted with GPS and loT devices, may coordinate grain transport between combines and storage locations, while smart scales at storage facilities may ensure accurate weighing and inventory tracking. All this data may be transmitted through wireless networks to a central system for aggregation.

Once collected, the system may engage in data integration and analysis, using real-time analytics to detect trends and predict grain conditions. Based on this analysis, a decision-making algorithm may recommend operational changes. These algorithms could take into account variables such as moisture, cracked grain, combine speed, dryer efficiency, fuel use, labor availability, and projected revenue. If certain thresholds are reached, such as moisture exceeding 15% or foreign material surpassing 2%, adjustments may be suggested to optimize harvester settings or harvesting order.

A detailed operational flow may begin with confirming sensor functionality and inputting key field parameters such as layout and expected quality goals. As harvesting starts, combine harvesters may gather and transmit data while trucks and grain carts report their location and load status. Grain quality measurements from various system points, combine, truck, scale, dryer, or storage, may feed into the central system. The system may then generate recommendations for adjustments to combine settings or harvesting sequence. For example, operators may be advised to move to another part of the field where lower moisture levels are predicted, reducing drying costs. These adjustments may continue throughout harvesting as new data is received. It might also be the goal of a smart harvesting grading system to connect the data from the loads arriving at a grain receiving location with the grading system on a grain cart or combine in order that the smart system recommends changed actions due to the constantly changing parameters. For example, if the grain is arriving dryer than the target moisture. The smart system might recommend that the combine harvest in a wetter portion of the field for part of the load to try to optimally blend one or more truckload.

In post-harvest analysis, smart harvesting may evaluate the effectiveness of its recommendations and use the data to improve future operations. Scenarios without smart trucks may still benefit from central recommendations and operator adjustments based on real-time quality feedback. However, when smart trucks are included, further optimization may occur. These vehicles may receive dynamic routing updates to minimize delays and integrate their transport data into the system for continuous analysis and logistical refinement. The smart system may be able to automatically route trucks to certain sites or storage structures to enable the most uniform of stored materials.

When machine learning and artificial intelligence (AI) are added to smart harvesting, predictive models may be trained using historical data to forecast optimal harvester settings, drying requirements, or ideal field routes. Clustering algorithms may segment fields into productivity zones based on soil conditions and environmental trends. As data flows in, AI may continuously refine its recommendations and help coordinate actions across harvesters, trucks, and grain facilities.

Additionally, on-board grain grading systems may be used. These on-board grain grading systems may use spectroscopy and optical sensors to assess grain quality, detecting issues like excess foreign material or suboptimal protein content. Upon detecting problems, the system may recommend immediate corrective actions, such as reducing speed or adjusting fan settings, to improve grain quality in real time. The resulting data may then be merged with insights from smart scales and storage systems to optimize the broader post-harvest process.

By integrating sensing technologies, real-time communication, machine learning, and AI, this Smart Harvesting System of Systems may support more informed, efficient, and adaptive decision-making. While specific outcomes may vary by operation and environmental conditions, the system may contribute to better grain quality, reduced operational costs, and more sustainable farming practices over time.

By way of another example, the systems and method disclosed herein may have applications relating to meat grading and sorting. An automated meat grading process may utilize including imaging systems, artificial intelligence (AI), and machine learning, to achieve consistent and efficient grading. High-resolution cameras and imaging sensors may capture detailed images of meat cuts as they move along production lines. These images may be analyzed by AI algorithms trained to detect quality indicators such as marbling, fat distribution, and color. Automated systems may also incorporate tactile sensors or pressure-sensitive devices to estimate firmness (e.g., meat tenderness) by mimicking the human sense of touch. In addition, ultrasound technology may be used to measure internal tissue density by analyzing how sound waves travel through the meat. These systems may deliver consistent measurements that align with predetermined quality benchmarks, potentially minimizing the subjectivity and variability associated with manual inspections.

Over time, automated systems may also learn from accumulating data, adapting to changes in industry standards or consumer preferences. Unlike manual processes, automated grading systems may operate continuously, helping to reduce labor bottlenecks and increase processing speed. Although manual grading may offer the benefit of human judgment and experience, it may also introduce inconsistency due to fatigue or personal bias. In contrast, automation may standardize grading decisions, increasing reliability and throughput.

When paired with automated meat sorting, these grading systems may further streamline operations. Grading data may be instantly communicated to sorting mechanisms, such as robotic arms or intelligent conveyor belts, which may direct each cut to a specific packaging line or storage bin based on its grade. This real-time coordination may reduce manual handling errors, maintain grading integrity, and support precise product categorization. Higher-grade cuts may be allocated to premium retail packaging, while lower grades may be routed for bulk sales or further processing. Additionally, sorting systems may collect performance data that supports operational analysis, inventory management, and strategic planning.

The integration of automated meat traceability may also enhance food safety, transparency, and regulatory compliance. Each meat cut may be linked to a unique carcass identifier, via barcodes or RFID tags, which may enable traceability throughout the production chain. This data may include information about the animal's origin, the producer's location and farming practices, and any health treatments administered. During processing, the system may log dates, handling methods, and quality control checkpoints. Certification labels such as “organic” or “grass-fed” may also be associated with individual cuts, supporting consumer trust and marketing efforts. In cases of safety concerns or recalls, traceability data may allow companies to quickly isolate affected products and comply with food safety regulations.

Together, these technologies may create a highly integrated system that enhances consistency, reduces manual variability, and supports transparency and efficiency in the meat production process. While implementation may depend on operational capacity, investment, and regulatory context, the potential value in quality control, consumer trust, and operational optimization may be significant.

By way of another example, the systems and method disclosed herein may have applications relating to fruit and vegetable monitoring and grading. This may be completed also with odor sensors. These odor sensors are designed to emulate the human sense of smell by using arrays of odor sensors that detect volatile organic compounds (VOCs). The data from these odor sensors may be processed using machine learning algorithms to recognize complex chemical signatures. Odor sensors may be able to detect a variety of substances, including esters, acetic acid, butyric acid, natural toxins such as and mycotoxins, chemical residues from pesticides like chlorpyrifos or herbicides such as glyphosate, as well as industrial pollutants like ammonia, benzene, and hydrogen sulfide. In some cases, odor sensors may be able to identify substances like illicit drugs or explosives.

Some decaying fruits emit methanethiol and dimethyl sulfide, both of which have intensely foul odors. Bacteria, (e.g., Acetobacter), can oxidize ethanol into acetic acid, producing a pungent, vinegar-like smell. Overripe or fermenting fruit often releases a sweet, boozy aroma, similar to wine, cider, or beer, due to yeast fermentation converting sugars into ethanol and carbon dioxide. Fungi (e.g., Penicillium, Aspergillus, or Botrytis) that grow on the fruit's surface can create musty, earthy, or dusty odors, indicating spoilage. Even before fruit begins to rot, very ripe specimens may develop a cloying, syrupy scent from esters like isoamyl acetate and ethyl butyrate, which can become nauseating at high concentrations. Bananas may initially emit sweet esters (e.g., isoamyl acetate) which may give off the characteristic “fake banana” smell, followed by sour and alcoholic notes as they decay. Apples may begin with a fruity, cider-like aroma that shifts to a vinegary scent, and eventually, a musty odor associated with decay. Citrus fruits may develop moldy and sharply sour smells, and in some cases, a kerosene-like odor due to the degradation of limonene. Grapes may produce a wine-like aroma as they ferment, which can evolve into the sharp scent of acetic acid and sulfurous compounds. Melons may start off with a sickly sweet smell that later turns into rancid odors from fatty acids and sulfur compounds as they spoil. Odor sensors may be supported by odor signature databases, which, in turn, may be supported by big data and artificial intelligence. By aggregating chemical profiles from various food products and known contaminants, models may be created that predict the presence of harmful substances based on specific odor patterns.

Odor sensors may be calibrated in manufacturing environments to detect selected odors or combinations of odors that are linked to spoilage, contamination, or other quality concerns.

Conventional detection methods in food processing often rely on laboratory-based analyses that require significant time and manual labor, which can delay corrective actions. In contrast, the integration of odor sensors may provide real-time detection of spoilage or contamination, allowing for faster responses that reduce waste or prevent public health risks. These odor sensors may offer economic benefits as well. Odor sensors currently cost only a few hundred dollars in some cases, and while optical or thermal imaging equipment may be more expensive, the cost may be justified when spread across large volumes of agricultural produce, potentially making these systems viable for commercial use.

Odor sensors may be integrated with hyperspectral, thermal, and optical imaging technologies to study and classify odor patterns associated with fruits, vegetables, and broader food commodities. VOC sensors may be trained to detect early signals of spoilage or degradation. In the context of fresh produce, such technology may enable early detection of microbial growth during storage or shipment, allowing for proactive interventions before spoilage becomes visible. This could be particularly valuable in reducing waste and improving food quality throughout the supply chain.

Odor sensors may also assist in monitoring ripeness by detecting when fruits are nearing or have passed their optimal freshness, which could help retailers better manage inventory. Similarly, by identifying early signs of deterioration, predictive maintenance actions may be taken to preserve quality and reduce losses. During transportation, odor monitoring systems may provide real-time assessments to ensure that goods remain within acceptable quality parameters, potentially minimizing rejected loads and associated financial losses.

In retail settings, data from odor sensors may support more informed decisions about pricing and product placement, potentially helping managers determine which items to prioritize for sale. From a food safety standpoint, early detection of harmful bacteria or mold (e.g., before it is visible) may reduce the risk of foodborne illnesses. The odor sensors may also support strategies to extend shelf life by signaling when storage conditions should be modified, such as adjusting temperature or humidity to delay spoilage. It is contemplated that low-cost odor sensing technologies may be utilized in the transport of produce, either in the storage container, crate, and even as a sticker applied to each piece of higher value fruit to enable easy sorting and to prevent spoilage. Using these technologies, it would be possible to segregate fruit by spoilage date and use up older or faster degrading produce sooner. Used in transport and in supermarket operations, smart technologies can assist to reduce the disposal of high value produce. The smart stickers are intended to change color when a chemical emitted from fruit such as pineapples, such as ethylene is present in higher concentrations that correspond to less or more desirable taste.

As odor sensors collect data over time, the insights gained may be used to analyze trends in spoilage, ripeness, or other quality indicators. This information may help optimize supply chain practices and improve inventory turnover strategies. For consumers, technologies that provide real-time freshness information (whether through packaging or in-store displays) may increase confidence in product quality. Finally, by reducing spoilage and improving efficiency, such systems may contribute to environmental sustainability by decreasing food waste and the greenhouse gas emissions associated with decomposing organic matter.

Referring now broadly to the controller 114, the processors 116, the memory 118, and the user interface 120, aspects of the controller 114, the processors 116, the memory 118, and the user interface 120 are further discussed.

It is noted that any components recited in the present application may have their own controller 114, processors 116, memory 118, and/or user interface 120. Additionally, one or more components recited in the present application may share a common controller 114, processors 116, memory 118, and/or user interface 120. Further, the controller 114, the processors 116, the memory 118, and the user interface 120 may be located onboard any of the components recited in the present application or may be located remotely (e.g., via a wireless connection) from any of the components recited in the present application.

The controller 114 may be located onboard any system disclosed herein. For example, if the controller 114 is located onboard any system disclosed herein the controller may be able to access a server (e.g., over a wireless connection or a wired connection). Additionally, the controller 114 may be located remotely from any system disclosed herein. For example, the controller 114 may connect by a wireless connection.

The one or more processors 116 of a controller 114 may include any processor or processing element known in the art. For the purposes of the present disclosure, the term “processor” or “processing element” may be broadly defined to encompass any device having one or more processing or logic elements (e.g., one or more micro-processor devices, one or more application specific integrated circuit (ASIC) devices, one or more field programmable gate arrays (FPGAs), or one or more digital signal processors (DSPs)). In this sense, the one or more processors 116 may include any device configured to execute algorithms and/or instructions (e.g., program instructions stored in memory). In embodiments, the one or more processors 116 may be embodied as a desktop computer, mainframe computer system, workstation, image computer, parallel processor, networked computer, or any other computer system configured to execute a program configured to operate or operate in conjunction with any system disclosed herein, as described throughout the present disclosure. Moreover, different subsystems of any system disclosed herein may include a processor or logic elements suitable for carrying out at least a portion of the steps described in the present disclosure. Therefore, the above description should not be interpreted as a limitation on the embodiments of the present disclosure but merely as an illustration. Further, the steps described throughout the present disclosure may be carried out by a single controller or, alternatively, multiple controllers. Additionally, the controller 114 may include one or more controllers housed in a common housing or within multiple housings. In this way, any controller or combination of controllers may be separately packaged as a module suitable for integration into any system disclosed herein.

The memory 118 may include any storage medium known in the art suitable for storing program instructions executable by the associated one or more processors 116. For example, the memory 118 may include a non-transitory memory medium. By way of another example, the memory 118 may include, but is not limited to, a read-only memory (ROM), a random-access memory (RAM), a magnetic or optical memory device (e.g., disk), a magnetic tape, a solid-state drive, and the like. It is further noted that the memory 118 may be housed in a common controller housing with the one or more processors 116. In some embodiments, the memory 118 may be located remotely with respect to the physical location of the one or more processors 116 and the controller 114. For instance, the one or more processors 116 of the controller 114 may access a remote memory (e.g., server), accessible through a network (e.g., internet, intranet, and the like).

In embodiments, the user interface 120 may include, but is not limited to, one or more desktops, laptops, tablets, and the like. In another embodiment, the user interface 120 includes a display used to display data of any system disclosed herein to a user. The display of the user interface 120 may include any display known in the art. For example, the display may include, but is not limited to, a liquid crystal display (LCD), an organic light-emitting diode (OLED) based display, or a CRT display. Those skilled in the art should recognize that any display device capable of integration with a user interface 120 is suitable for implementation in the present disclosure. In embodiments, a user may input selections and/or instructions responsive to data displayed to the user via a user input device of the user interface 120.

The herein described subject matter sometimes illustrates different components contained within, or connected with, other components. It is to be understood that such depicted architectures are merely exemplary, and that in fact many other architectures can be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermedial components. Likewise, any two components so associated can also be viewed as being “connected” or “coupled” to each other to achieve the desired functionality, and any two components capable of being so associated can also be viewed as being “couplable” to each other to achieve the desired functionality. Specific examples of couplable include but are not limited to physically interactable and/or physically interacting components and/or wirelessly interactable and/or wirelessly interacting components and/or logically interactable and/or logically interacting components.

It is believed that the present disclosure and many of its attendant advantages will be understood by the foregoing description, and it will be apparent that various changes may be made in the form, construction, and arrangement of the components without departing from the disclosed subject matter or without sacrificing all of its material advantages. The form described is merely explanatory, and it is the intention of the following claims to encompass and include such changes. Furthermore, it is to be understood that the invention is defined by the appended claims.

1. A grading system, comprising:
one or more sampling probes;
one or more sensors, wherein the one or more sensors are configured to collect information of a material within a container, wherein the one or more sampling probes deliver the material to the one or more sensors, wherein the one or more sensors position the one or more sampling probes relative to the container;
a support structure, wherein the support structure is configured to position the one or more sampling probes above the container;
one or more motors, wherein the one or more motors are coupled to the one or more sampling probes in order to actuate the one or more sampling probes; and
a controller communicatively coupled to the one or more sensors and the one or more motors, the controller including one or more processors configured to execute a set of program instructions stored in memory, the set of program instructions configured to cause the one or more processors to:
cause the one or more motors to actuate the one or more sampling probes such that the one or more sampling probes are inserted into the container at one or more locations to collect the information of the material within the container;
receive the information collected by the one or more sensors; and
grade the material within the container based on the information collected by the one or more sensors.

2. The grading system of claim 1, wherein the one or more sensors includes an odor sensor configured to detect an odor signature of the material.

3.-5. (canceled)

6. The grading system of claim 2, wherein the one or more processors are further configured to:
determine if the odor signature for the material is within selected bounds; and
provide an alert if the odor signature is not within the selected bounds.

7. The grading system of claim 1, wherein the one or more sensors includes an optical sensor configured to collect optical data of the material.

8. The grading system of claim 7, wherein the one or more processors are further configured to:
determine, based on the optical data, if the material is a correct type of material; and
provide an alert if the type of material is incorrect.

9. The grading system of claim 7, wherein the optical data of the material indicates at least one of defects in the material, discoloration of the material, material damage, or foreign material within the material.

10. The grading system of claim 9, wherein the one or more processors are further configured to:
determine, based on at least one of the defects in the material, the discoloration of the material, the material damage, or the foreign material within the material, if the optical data for the material is within selected bounds; and
provide an alert if the optical data is not within the selected bounds.

11. The grading system of claim 1, wherein the one or more sensors includes a moisture sensor to measure a moisture content of the material.

12. The grading system of claim 1, wherein the support structure comprises:
one or more arms actuated with the one or more motors to control the one or more sampling probes, wherein the one or more sampling probes are positioned on an end of one of the one or more arms.

13. The grading system of claim 1, wherein the support structure comprises:
one or more vertical members, wherein at least one of the one or more vertical members are located on a side of the container from each other; and
one or more horizontal members, wherein each of the one or more horizontal members are coupled to of the at least one of the one or more vertical members, wherein the one or more horizontal members spans the container, wherein the one or more sampling probes are coupled to the one or more horizontal members, wherein the one or more motors provide motion for the one or more sampling probes in at least two dimensions along the horizontal member.

14. The grading system of claim 13, wherein the support structure is static such that the container is positioned under the support structure.

15. The grading system of claim 13, wherein the support structure is mobile such that the support structure is positioned over the container.

16. The grading system of claim 1, wherein the one or more sampling probes are programmed to insert into the container at at least one of a pre-programmed location or random locations.

17.-20. (canceled)

21. The grading system of claim 1, wherein the one or more sampling probes are configured to remove more than one sample of the material from the container each time the one or more sampling probes are inserted into the container, wherein each sample of the more than one samples is collected from a different location within the container.

22. The grading system of claim 1, wherein the container is scanned by the one or more sensors to determine if there are any obstructions before inserting the sampling probe into the container.

23.-37. (canceled)

38. A continuous grading system, comprising:
a weighing device, wherein the weighing device weighs a material to calculate a bulk density;
an optics device, wherein the optics device is configured to capture one or more images of the material as the material passes through the optics device;
a moisture sensing device, wherein the moisture sensing device determines a moisture content of the material based on a cross section of the material;
an odor sensing device, wherein the odor sensing device determines an odor profile of the material;
one or more valves, wherein the one or more valves are configured to control movement of the material, wherein the one or more valves are configured to allow the material to flow without an external input; and
a controller communicatively coupled to the weighing device, the optics device, the moisture sensing device, an odor sensing device, and the one or more valves, the controller including one or more processors configured to execute a set of program instructions stored in memory, the set of program instructions configured to cause the one or more processors to:
receive weight of the material, the one or more images of the material, the moisture content of the material, and the odor profile of the material; and
grade the material based on the one or more images of the material, the moisture content of the material, and the odor profile of the material.

39. (canceled)

40. The continuous grading system of claim 38, further comprising:
a separation device, wherein the separation device comprises:
a first tube;
a second tube, wherein the first tube and the second tube are coupled such that the second tube enters the first tube at an angle relative to the first tube, wherein the second tube introduces a combination of the material and foreign material to the first tube; and
a fan, wherein the fan is configured to blow a gas at a selected velocity, wherein the gas separates the material and the foreign material, wherein the gas directs the foreign material in one direction of the first tube and the material falls in a second direction of the first tube.

41. The continuous grading system of claim 38, wherein the weighing device comprises:
a fixed volume, wherein the fixed volume is configured to be filled with the material such that the material entirely fills the fixed volume without compressing the material;
a bottom valve, wherein the bottom valve is configured to prevent the material from exiting the fixed volume; and a top valve cuts the material off, to ensure that the fixed volume is fully filled with material, without compressing the material itself; and
one or more load cells, wherein the one or more load cells are coupled to the fixed volume such that the one or more load cells weigh the material when the material entirely fills the fixed volume without being compressed.

42. The continuous grading system of claim 38, wherein the optics device comprises:
a chamber, wherein the chamber is configured to allow the material to fall through the chamber;
a valve, wherein the valve is configured to be opened to a predetermined amount depending on a type of material to be imaged;
one or more cameras, wherein the one or more cameras are coupled to a wall of the chamber, wherein the one or more cameras are configured to image the material inside the chamber; and
lighting, wherein the lighting is positioned within the chamber and are configured to illuminate within the chamber, wherein the lighting surrounds the one or more cameras and the one or more lights are synchronized to illuminate the chamber with the one or more cameras.

43.-49. (canceled)

50. The continuous grading system of claim 38, further comprising:
a mass estimating device, wherein the mass estimating device comprises:
a tube, wherein the tube contains the material;
a blower, wherein the blower is configured to blow a gas at a selected velocity in order to deflect the material within the tube;
one or more sensors, wherein the one or more sensors detect the material deflected within the tube and measure a displacement of the material and individual particles within the material; and
a controller communicatively coupled to the blower and the one or more sensors, the controller including one or more processors configured to execute a set of program instructions stored in memory, the set of program instructions configured to cause the one or more processors to:
receive the displacement of at least one of the material of the individual particles within the material from the one or more sensors; and
estimate mass of at least one of the material or the individual particles within the material based on the displacement of the material.

51.-122. (canceled)
